SEQUENCE LISTING (52 sequences)

SEQ ID NO: 1 - DNA, 21 bp, Artificial Sequence
  Description of Artificial Sequences; note = synthetic construct
dgcagagcagtgtcctcctca

SEQ ID NO: 2 - DNA, 23 bp, Artificial Sequence
  Description of Artificial Sequences; note = synthetic construct
dggacatactggccatcaggaga

SEQ ID NO: 3 - DNA, 21 bp, Artificial Sequence
  Description of Artificial Sequences; note = synthetic construct
dagattgcatgtcccctggaa

SEQ ID NO: 4 - DNA, 21 bp, Artificial Sequence
  Description of Artificial Sequences; note = synthetic construct
dcattgaatggcgtggattca

SEQ ID NO: 5 - DNA, 23 bp, Artificial Sequence
  Description of Artificial Sequences; note = synthetic construct
dtggccatcagattgtcacagac

SEQ ID NO: 6 - DNA, 22 bp, Artificial Sequence
  Description of Artificial Sequences; note = synthetic construct
dccagccaataaagatggcagg

SEQ ID NO: 7 - DNA, 21 bp, Artificial Sequence
  Description of Artificial Sequences; note = synthetic construct
dcgcagctcaggaagaatgtg

SEQ ID NO: 8 - DNA, 23 bp, Artificial Sequence
  Description of Artificial Sequences; note = synthetic construct
dtgaagtacactggcattgacga

SEQ ID NO: 9 - DNA, 23 bp, Artificial Sequence
  Description of Artificial Sequences; note = synthetic construct
dacccgaggagaagaggagtttg

SEQ ID NO: 10 - DNA, 23 bp, Artificial Sequence
  Description of Artificial Sequences; note = synthetic construct
dgcttctttcccagtgacaagca

SEQ ID NO: 11 - DNA, 22 bp, Artificial Sequence
  Description of Artificial Sequences; note = synthetic construct
daagacagcagccccagtgaat

SEQ ID NO: 12 - DNA, 23 bp, Artificial Sequence
  Description of Artificial Sequences; note = synthetic construct
dtcggtcaaagaggtcacagagg

SEQ ID NO: 13 - DNA, 21 bp, Artificial Sequence
  Description of Artificial Sequences; note = synthetic construct
dtcttccccagcgatggtttt

SEQ ID NO: 14 - DNA, 20 bp, Artificial Sequence
  Description of Artificial Sequences; note = synthetic construct
dttgctcttcttgccctcga

SEQ ID NO: 15 - DNA, 23 bp, Artificial Sequence
  Description of Artificial Sequences; note = synthetic construct
dgtctgcctggagattctgttcg

SEQ ID NO: 16 - DNA, 22 bp, Artificial Sequence
  Description of Artificial Sequences; note = synthetic construct
dtcacggacacaaacatgaccc

SEQ ID NO: 17 - DNA, 22 bp, Artificial Sequence
  Description of Artificial Sequences; note = synthetic construct
dgacgtctttcccgatattcgg

SEQ ID NO: 18 - DNA, 22 bp, Artificial Sequence
  Description of Artificial Sequences; note = synthetic construct
dccacggcatccttgtcttttc

SEQ ID NO: 19 - DNA, 23 bp, Artificial Sequence
  Description of Artificial Sequences; note = synthetic construct
dgtgcctcaagaaatgtgccact

SEQ ID NO: 20 - DNA, 22 bp, Artificial Sequence
  Description of Artificial Sequences; note = synthetic construct
dacagaggaatccgctgcattc

SEQ ID NO: 21 - DNA, 26 bp, Artificial Sequence
  Description of Artificial Sequences; note = synthetic construct
rggacacaaaggacucucgacccaaa

SEQ ID NO: 22 - DNA, 26 bp, Artificial Sequence
  Description of Artificial Sequences; note = synthetic construct
ruuugggucgagaguccuuugugucc

SEQ ID NO: 23 - DNA, 26 bp, Artificial Sequence
  Description of Artificial Sequences; note = synthetic construct
raaauucacacuccucuucuggaggg

SEQ ID NO: 24 - DNA, 26 bp, Artificial Sequence
  Description of Artificial Sequences; note = synthetic construct
rcccuccagaagaggagugugaauuu

SEQ ID NO: 25 - DNA, 21 bp, Artificial Sequence
  Description of Artificial Sequences; note = synthetic construct
daatggccaccatggagaaca

SEQ ID NO: 26 - DNA, 23 bp, Artificial Sequence
  Description of Artificial Sequences; note = synthetic construct
daccacaattctgtctttcacgg

SEQ ID NO: 27 - DNA, 23 bp, Artificial Sequence
  Description of Artificial Sequences; note = synthetic construct
dcggatgaaactctgagcaatgt

SEQ ID NO: 28 - DNA, 22 bp, Artificial Sequence
  Description of Artificial Sequences; note = synthetic construct
dctgcagttctgtgagccaaag

SEQ ID NO: 29 - DNA, 23 bp, Artificial Sequence
  Description of Artificial Sequences; note = synthetic construct
dgcagagcagtttgtcctcctca

SEQ ID NO: 30 - DNA, 23 bp, Artificial Sequence
  Description of Artificial Sequences; note = synthetic construct
dggacatactggccatcaggaga

SEQ ID NO: 31 - DNA, 23 bp, Artificial Sequence
  Description of Artificial Sequences; note = synthetic construct
dgctttgtcctctctttgtgcca

SEQ ID NO: 32 - DNA, 21 bp, Artificial Sequence
  Description of Artificial Sequences; note = synthetic construct
dggccatcatccatgctgaag

SEQ ID NO: 33 - DNA, 23 bp, Artificial Sequence
  Description of Artificial Sequences; note = synthetic construct
dagtgctcaaggacatcgagacg

SEQ ID NO: 34 - DNA, 22 bp, Artificial Sequence
  Description of Artificial Sequences; note = synthetic construct
dagccacttctgcacattgctg

SEQ ID NO: 35 - DNA, 23 bp, Artificial Sequence
  Description of Artificial Sequences; note = synthetic construct
dtggaacagccaggtgtactgca

SEQ ID NO: 36 - DNA, 23 bp, Artificial Sequence
  Description of Artificial Sequences; note = synthetic construct
dtcttcggtagacgcacatgtcc

SEQ ID NO: 37 - DNA, 25 bp, Artificial Sequence
  Description of Artificial Sequences; note = synthetic construct
dgggccactgtcgcatcatgattgg

SEQ ID NO: 38 - DNA, 32 bp, Artificial Sequence
  Description of Artificial Sequences; note = synthetic construct
dtgtagctgttgcaaatgctttaaggaagaag

SEQ ID NO: 39 - DNA, 21 bp, Artificial Sequence
  Description of Artificial Sequences; note = synthetic construct
dcgcagctcaggaagaatgtg

SEQ ID NO: 40 - DNA, 23 bp, Artificial Sequence
  Description of Artificial Sequences; note = synthetic construct
dtgaagtacactggcattgacga

SEQ ID NO: 41 - DNA, 23 bp, Artificial Sequence
  Description of Artificial Sequences; note = synthetic construct
dccactgctcgtaaagacattcc

SEQ ID NO: 42 - DNA, 23 bp, Artificial Sequence
  Description of Artificial Sequences; note = synthetic construct
daccaattgcagaagactcaagc

SEQ ID NO: 43 - DNA, 22 bp, Artificial Sequence
  Description of Artificial Sequences; note = synthetic construct
dcttgctcttggacaggaacca

SEQ ID NO: 44 - DNA, 23 bp, Artificial Sequence
  Description of Artificial Sequences; note = synthetic construct
daccgagatgatgtagccagcag

SEQ ID NO: 45 - DNA, 23 bp, Artificial Sequence
  Description of Artificial Sequences; note = synthetic construct
dgcagatgctgtattttgcacct

SEQ ID NO: 46 - DNA, 22 bp, Artificial Sequence
  Description of Artificial Sequences; note = synthetic construct
dtctgccacatggtaaggcata

SEQ ID NO: 47 - DNA, 22 bp, Artificial Sequence
  Description of Artificial Sequences; note = synthetic construct
dgccaacaaagctcaggacaac

SEQ ID NO: 48 - DNA, 23 bp, Artificial Sequence
  Description of Artificial Sequences; note = synthetic construct
dgcagtgacttcgtcatttggac

SEQ ID NO: 49 - DNA, 21 bp, Artificial Sequence
  Description of Artificial Sequences; note = synthetic construct
dttggaaggcagcatttgcag

SEQ ID NO: 50 - DNA, 22 bp, Artificial Sequence
  Description of Artificial Sequences; note = synthetic construct
dtgtacggaatgcgtttcttgc

SEQ ID NO: 51 - DNA, 23 bp, Artificial Sequence
  Description of Artificial Sequences; note = synthetic construct
dgagacccgctgaacaataccac

SEQ ID NO: 52 - DNA, 23 bp, Artificial Sequence
  Description of Artificial Sequences; note = synthetic construct
dctggatcaagacccctcctttc

(19) United States
(12) Patent Application Publication
(10) Pub. No.: US 2007/0231822 A1
(43) Pub. Date: Oct. 4, 2007

(54) Methods for the detection and treatment of cancer

(76) Inventor: Michael Mitas, Moncks Corner, SC (US)

Correspondence Address: NEEDLE & ROSENBERG, P.C., SUITE 1000, 999 PEACHTREE STREET, ATLANTA, GA, 30309-3915, US

(21) Appl. No.: 11/711,493

(22) Filed: Feb. 27, 2007

Related U.S. Application Data
(60) Provisional application No. 60/777,402, filed on Feb. 28, 2006.
(60) Provisional application No. 60/784,009, filed on Mar. 20, 2006.

(51) Int. Cl. C12Q 1/68

(52) U.S. Cl. 435/6

(57) ABSTRACT

Methods are provided for the detection of and determining prognosis of metastatic breast, lung, prostate, and/or pancreatic cancer using various genetic markers, including markers for gene clusters linked by Esx. In one method, breast cancer micrometastases and non-small cell lung cancer metastases or micrometastases are detected in a patient by determining whether the AGR2 or TFF1 genes are overexpressed in a cell sample compared to control lymph node tissue cells. In a further method, the likelihood that a patient diagnosed with breast cancer will respond to hormonal therapy is predicted by determining a higher expression level of the AGR2 gene compared to a control gene. In a further method, a decreased probability of survival for a patient diagnosed with early stage non-small cell lung cancer is predicted by determining a higher expression level of the AGR2 gene compared to a control gene. Kits for practicing the methods of the invention are further provided. Methods are also provided for the identification of markers for which overexpression is indicative of the presence of micrometastatic disease.

### CROSS REFERENCE TO RELATED APPLICATIONS

This application claims priority to U.S. provisional application No. 60/777,402, filed on Feb. 28, 2006, and U.S. provisional application No. 60/784,009, filed on Mar. 20, 2006. The aforementioned applications are herein incorporated by this reference in their entireties.

### ACKNOWLEDGEMENTS

This invention was made with government support under grant numbers 1R21CA097875 and 7R33CA097875-02 awarded by the National Institutes of Health. The United States government has certain rights in the invention.

### BACKGROUND OF THE INVENTION

1. Field of the Invention

The invention relates to the field of molecular biology, particularly to the use of genetic markers in the detection, in determining prognosis, and in the treatment of various cancers, more particularly to the detection, determining prognosis, and treatment of metastatic lung, breast, pancreatic, and prostate cancer disease.

2. Background Art

The development of metastatic disease is by far the most common cause of death in cancer patients and results from dissemination of malignant cells throughout the body. The process whereby epithelial cancer cells gain metastatic potential is an extremely complex, highly organized, and non-random process. Although significant heterogeneity exists between different cancer types, common molecular pathways regulate the development of their metastatic potentials. The first step of the metastatic process involves the loss of epithelial characteristics, including cell-cell interactive properties and cellular polarity, which leads to invasion or migration of the neoplastic cells into adjacent stromal tissue. This epithelial-mesenchymal transition (EMT) appears to be initiated by a variety of signals such as transforming growth factor (TGF) β, the transcription factor twist, and oncogenic Ras (Bates & Mercurio (2005) Cancer Biol. Ther., 4:365-370; Yang et al. (2004) Cell, 117:927-939; Huber & Kraut (2005) Curr. Opin. Cell Biol., 17:548-558). EMT is subsequently maintained by autocrine factors of the epidermal growth family, fibroblast growth factors (FGF), human growth factor and insulin growth factors 1 and 2. A primary hallmark of EMT is downregulation and/or degradation of epithelial-specific cadherin (E-cadherin), a protein whose extracellular domain forms Ca²⁺-dependent homophilic trans-dimers, providing specific interaction with adjacent cells, and whose cytoplasmic domain is connected to the actin cytoskeleton via anchor proteins called catenins (Hogan et al. (2004) Mol. Cell. Biol., 24:6690-6700). Down regulation of E-cadherin contributes both to the loss of cellular polarity and cell-cell interactions.

To colonize a distant organ, invasive cells must next undergo a mesenchymal to epithelial conversion (MEC), a process that is exemplified by formation of nephronic tubules during normal development, and which has long served as a paradigm of understanding inductive signaling in morphogenesis (Levashova et al. (2003) Kidney Int., 63:2075-2087). MEC is activated by fibroblast growth factor 2 (FGF2), leukemia inhibitory factor, and TGF13-2, resulting in increased expression of epithelial-specific markers such as E-cadherin, cytokeratins, y-glutamyl-transpeptidase, and secreted frizzled-related protein 2 (Levashova et al. (2003) Kidney Int., 63:2075-2087; Barasch et al. (1999) Cell, 99:377-386; Barasch (2001) Curr. Opin. Nephrol. Hypertens., 10:429-436).

Metastatic disease can affect a number of organs and organ systems. For example, lung cancer is the leading cause of death from cancer in North America. For early stage (IA, IB, IIA, and IIIB) non-small cell lung cancer (NSCLC), surgical resection is the treatment of choice, but the death rate at 5 years is 20% to 40%. Until recently, the addition of postoperative adjuvant therapy to combat occult metastatic disease has been unsuccessful. However, recent studies with newer chemotherapeutic agents (Strauss et al. (2004) J. Clin. Oncol. 22:7019; Winton et al. (2005) N. Engl. J. Med. 352:2589-2597) have led to a new treatment paradigm with the recommendation that a brief course of chemotherapy should become the standard of care for patients with good performance status after complete resection of stage IB or stage II NSCLC. It is also possible that adjuvant chemotherapy would be beneficial in a subset of stage IA NSCLC patients. In many studies, increasing size of IA tumors is matched by decreasing survival (Mikhitarian et al. (2004) Biotechniques 36:474-478; Endoh et al. (2004) J. Clin. Oncol. 22:811819; Wisnivesky et al. (2004) Chest 126:761-765). Stage IA tumors exhibiting positive immunohistochemical markers reflecting molecular changes associated with tumor aggressiveness reportedly predict decreased patient survival (Okada et al. (2005) J. Thorac. Cardiovasc. Surg. 129:87-93). However, at present there is no reliable and easy method to ascertain which early-stage resected patients would benefit the most from undergoing adjuvant systemic therapy. Such a successful method would justify to patients and physicians the added toxicity of adjuvant chemotherapy and maximize survival benefit.

Breast cancer is second only to lung cancer as the leading cause of cancer death in women today. The potential for positive impact on the lives of women with breast cancer lies in the development of more sensitive methods of detection for improved diagnosis and prognosis, treatment modalities, and long term reduction in morbidity and mortality. The presence of metastatic disease in axillary lymph nodes (ALN) is considered the single most important prognostic indicator for breast cancer patients, with an inverse relationship existing between the number of lymph nodes positive for cancer and prognosis (Saimura et al. (1999) J Surg. Oncol. 71:101-105; Fisher et al. (1981) Surg. Gynecol. Obstet. 152:765-772). Although approximately 70% of hematoxylin and eosin (H&E) node-negative (node−) patients have demonstrated long-term disease free status (Gardner and Feldman (1993) Ann. Surg. 218:270-278), it has been shown that H&E staining methods may miss 9-25% of disease not detected by standard H&E (otherwise referred to as “micrometastases”) (Cote et al. (1999) Lancet 354:896-900; International (Ludwig) Breast Cancer Study Group (1990) Lancet 335:1565-1568), Recently it has been observed that the addition of cytokeratin immunohistochemical (IHC) staining to routine single H&E evaluation can produce an enhancement of disease detection by 10-25% (e.g., Cote et al. (1999) Lancet 354:896-900; de Mascarel et al. (1992) Br. J. Cancer 66:523-527; McGuckin et al. (1996) Br. J. Cancer 73:88-95). The potential for positive impact on the lives of women with breast cancer lies in the development of more sensitive methods of detection for improved diagnosis and prognosis, treatment modalities, and long term reduction in morbidity and mortality.

The present invention solves needs for providing more sensitive methods for the detection, diagnosis, determining prognosis, and treatment of various cancers, including metastatic lung, breast, pancreatic, and prostate cancer disease.

### SUMMARY OF THE INVENTION

Methods are provided for detecting breast cancer in a patient and for predicting the likelihood that a breast cancer patient will respond to hormonal therapy. In one method, breast cancer micrometastases are detected in a patient by obtaining a cell sample suspected of containing cancerous cells from axillary lymph nodes tissue, in particular sentinel lymph node tissue, and determining whether the AGR2 or TFF1 genes are overexpressed in the cell sample compared to control lymph node tissue cells. Preferred control lymph node tissue includes, for example, cervical lymph node tissue. In a further method, the likelihood that a patient diagnosed with breast cancer will respond to hormonal therapy is predicted by determining the expression level of the AGR2 gene in a cell sample containing primary, metastatic, or micrometastatic breast cancer cells, in particular from axillary or sentinel lymph node tissue, where a higher expression level of the AGR2 gene compared to the expression level of a control gene in that cell sample is indicative of an increased likelihood of response to treatment with hormonal therapy. In a preferred method, the comparison of expression levels is expressed as a ratio of AGR2 gene expression compared to control gene expression. Preferred control genes include, for example, TFF 1 or EpCam. Gene expression can be assessed at the protein or nucleic acid level. These methods may be carried out using cell samples obtained from tissue that is fixed, paraffin-embedded, fresh, or frozen.

Methods are also provided for detecting non-small cell lung cancer in a patient and for evaluating the prognosis of a patient with this disease. In one method, non-small cell lung cancer metastases or micrometastases are detected in a patient by obtaining a cell sample suspected of containing cancerous cells from mediastinal lymph node tissue and determining whether the AGR2 gene is overexpressed in the cell sample compared to control lymph node tissue cells. In another method, a decreased probability of survival for a patient diagnosed with early stage non-small cell lung cancer is predicted by determining the expression level of the AGR2 gene in a cell sample containing primary, metastatic, or micrometastatic non-small cell lung cancer cells compared to the expression level of a control gene, where a higher expression level of the AGR2 gene compared to the expression level of the control gene in that cell sample is indicative of a decreased probability of survival. Gene expression can be assessed at the protein or nucleic acid level. In a preferred method, the determination of the expression level of the AGR2 gene is part of a real-time RT-PCR analysis of a multi-marker panel of genes, particularly where the multi-marker panel of genes includes measurement of expression of the EpCam gene, the PDEF gene, the S100P gene, or combinations thereof. Preferred control genes include, for example, β2-microglobulin. Preferred tissues from which cell samples are obtained include mediastinal lymph node tissue. These methods may also be carried out using cell samples obtained from tissue that is fixed, paraffin-embedded, fresh, or frozen.

Methods are also provided for the treatment of breast and non-small cell lung cancer. In one method, the growth of breast cancer cells or non-small cell lung cancer cells in human tissue is inhibited by contacting the tissue with an inhibitor that interacts with AGR2 protein, AGR2 DNA, or AGR2RNA. In particular, this method may involve inhibitors that include siRNA, miRNA, antisense RNA, antisense DNA, or antagonists of the AGR2 protein such as anti-AGR2 antibodies.

A kit is also provided comprising reagents for practicing the methods of the invention, including at least one PCR primer needed to perform an amplification of at least one of the target nucleic acids disclosed for use in the methods of the present invention.

Methods are also provided for identifying markers for the detection of micrometastatic disease. In one method, markers indicative of the presence of micrometastatic disease in a patient are identified using a dilutional microarray approach in which: 1) a plurality of candidate markers is selected; 2) a sample of RNA isolated from metastatic tissue is diluted into an excess of RNA isolated from non-metastatic tissue at a ratio of at least 1:50 to create a dilution sample; 3) the expression levels of the candidate markers are measured in the dilution sample, an undiluted sample of RNA isolated from metastatic tissue, and a sample of RNA isolated from non-metastatic tissue; and 4) a sub-set of markers is selected from the plurality of candidate markers in which an absence of expression was observed in the sample of RNA isolated from non-metastatic tissue, a fluorescence signal above 500 relative units was observed in the undiluted sample of RNA isolated from metastatic tissue, and a fluorescence signal was observed in the dilution sample; where overexpression of at least one of the markers in the selected sub-set of markers is indicative of the presence of micrometastatic disease in the patient.

Methods are also provided for detecting metastatic cancer in a patient by obtaining a cell sample suspected of containing cancerous cells from lymph node tissue and determining whether one or more genes in a multi-marker panel of genes are overexpressed in said cell sample compared to expression of said genes in control lymph node tissue cells, wherein overexpression of said genes is indicative of the presence of metastatic cancer in said patient, wherein said metastatic cancer is metastatic breast, lung, or pancreatic cancer. In one embodiment, the multi-marker panel of genes comprises the Esx gene. In another embodiment, the multi-marker panel of genes comprises the EpCAM1 gene, AGR2 gene, CK19 gene, or CK8 gene, or any combination thereof. In another embodiment, the multi-marker panel of genes comprises the Esx gene, the Map7 gene, the S100P gene, the AGR2 gene, the CEA6 gene, the GPX2 gene, the TFF1 gene, the Mal2 gene, the Spint2 gene, the EpCAM1 gene, the EpCAM2 gene, the CK8 gene, the CK19 gene, or the Claudin3 gene, or any combination thereof. In another embodiment, the multi-marker panel comprises the EpCAM1 gene, the EpCAM2 gene, the AGR2 gene, the Esx gene, the CK19 gene, the CK8 gene, the CEA6 gene, or the Mal2 gene, or any combination thereof, in conjunction with a method for detecting metastatic non-small cell lung cancer. In another embodiment, the multi-marker panel of genes comprises the AGR2 gene, the S100P gene, the CK19 gene, the NQ01 gene, the MET gene, the MAGE-A6 gene, the XAGE-1 gene, the KRTHB 1 gene, the MAGE-A3 gene, or the MAP7 gene, or any combination thereof, in conjunction with a method for detecting metastatic non-small cell lung cancer. In another embodiment, the multi-marker panel of genes comprises the AGR2 gene, the S100P gene, the CK19 gene, the NQ01 gene, the MET gene, the MAGE-A6 gene, the XAGE-1 gene, the KRTHB1 gene, the MAGE-A3 gene, or the MAP7 gene, or any combination thereof, in conjunction with a method for detecting metastatic non-small cell lung cancer. In another embodiment, the multi-marker panel of genes comprises the AGR2 gene, the S100P gene, the CK19 gene, the Mucin1 gene, the FXYD gene, the Claudin3 gene, the CEA6 gene, the GPCR5A gene, the CK7 related gene, or the SCNN1A gene, or any combination thereof, in conjunction with a method for detecting metastatic breast cancer. In another embodiment, the multi-marker panel of genes comprises the PNLIPRP2 gene, the CK19 gene, the AGR2 gene, the FXYD gene, the SGP28 gene, the CEA6 gene, the gene of Accession Number AB020676, the Mucin1 gene, the gene of Accession Number AB028949, or the MMP19 gene, or any combination thereof, in conjunction with a method for detecting metastatic pancreatic cancer.

Further provided is a method for detecting metastatic cancer in a patient, comprising obtaining a cell sample suspected of containing cancerous cells from lymph node tissue of said patient and determining whether the Esx gene is overexpressed in said cell sample compared to Esx gene expression in control lymph node tissue cells, wherein overexpression of the Esx gene is indicative of the presence of metastatic cancer in said patient, wherein said metastatic cancer is metastatic breast, lung, or pancreatic cancer.

Methods are also provided for the treatment of metastatic breast, lung, or pancreatic cancer comprising inhibiting the growth of metastatic cancer cells in human tissue by contacting the tissue with an inhibitor that interacts with Esx protein, Esx DNA, or Esx RNA and thereby inhibits Esx function. In particular, this method may involve inhibitors that include siRNA, miRNA, antisense RNA, antisense DNA, or antagonists of the Esx protein such as anti-Esx antibodies.

Further provided is a method for detecting prostate cancer in a patient, comprising a) obtaining a cell sample suspected of containing prostate cancer cells from a body fluid of the patient; and b) determining whether the EpCAM2 gene is overexpressed in the cell sample compared to a normal, control level of expression of the EpCAM2 gene expression in a corresponding body fluid sample, wherein overexpression of the EpCAM2 gene in the cell sample is indicative of the presence of prostate cancer in the patient. In one aspect, the body fluid can be blood, plasma, serum, or urine. In one aspect, the patient can have advanced prostate cancer. In another aspect, the patient can have clinically undetectable prostate cancer.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 shows real-time RT-PCR analysis of archived fresh frozen paraffin fixed (FFPE) pathology-positive and frozen pathology-negative marker-positive axillary lymph nodes from breast cancer patients. Real-time RT-PCR analysis was performed on: A) 9 archived paraffin embedded pathology-positive [H&E(+)]; and B) 72 frozen pathology-negative marker-positive [H&E(−)/PCR(+)] axillary lymph nodes from breast cancer patients. The chosen H&E(−)/PCR(+) were selected based on results with mam, PIP, mama, CEA, CK19, muc1, and PDEF genes. Overexpression is presented as fold expression beyond threshold. The number of samples positive for the particular gene in each data set is indicated as “n.”

FIG. 2 shows real-time RT-PCR analysis of AGR2 and CPB1. Real-time RT-PCR analysis was performed on three breast cancer cell lines (left side of panel (MDA231, open square; MDA361, filled circle; MDA 453, open circle), pathology positive lymph node (filled diamonds), and cervical control lymph node (open triangles).

FIG. 3 shows real-time RT-PCR analysis of select genes from the Genomic Health study (Paik et al. (2004) N Engl. J. Med. 351:2817-26). Real-time RT-PCR analysis was performed on 12 metastatic tissues (left side of panel, open diamond) and 10 cervical control lymph nodes (open triangles).

FIG. 4 shows the relative rate of overexpression of various genes in 29 axillary lymph nodes determined to express at least one gene at significantly elevated levels. Over expression was measured using real-time RT-PCR. AGR2 was overexpressed in 59% of the nodes.

FIG. 5 shows real-time RT-PCR analysis of NSCLC tissue embedded in paraffin. Real-time RT-PCR analyses of indicated samples were performed using primer pairs for the indicated genes. Cₜ values for each gene were determined from triplicate reactions. ΔCₜ values were obtained by subtracting the mean Cₜ value of β₂-microglobulin from the mean Cₜ value for each respective gene. Horizontal lines indicate ΔCₜ respective threshold values of marker positivity based on 2 or 3 standard deviations beyond the mean of normal controls.

FIG. 6 shows real-time RT-PCR analysis of NSCLC-associated genes. RNA was extracted from FFPE tissues (20 or 50μ sections), and real-time RT-PCR analyses of the samples were performed using primer pairs for the indicated genes. Cₜ values for each gene were determined from triplicate reactions. ΔCₜ values were obtained by subtracting the mean Cₜ value of β₂-microglobulin from the mean Cₜ value for each respective gene. The number of samples analyzed from each tissue type listed at the bottom of the figure are: control negative MLN (open circles; n=24) derived from lung transplant patients (n=15); primary tumors (“+”; n=30) derived from NSCLC patients (n=18); pathology-positive MLN (filled circles; n=27) from NSCLC patients (n=15); pathology-negative, IHC-negative MLN (open triangles; n=44) from NSCLC patients (n=27); pathology-negative, IHC-positive MLN (“X”; n=4) from NSCLC patients (n=2). Sections were determined to be IHC positive if signals were observed by using BerEp4 antibody and an anti-cytokeratin mix. XAG=AGR2.

FIG. 7 shows area under the curve (AUC) values for NSCLC-associated genes. AUC values for the indicated genes were determined using MedCalc software for macro- and micro-metastatic NSCLC disease as described in the text. The mean of the AUC values is shown, with error bars corresponding to the standard deviation. XAG=AGR2.

FIG. 8 shows a correlation map of genes associated with metastatic disease in breast, NSCLC, and pancreatic cancer. The correlation map was constructed using criteria described in the text. Genes are positioned in a hypothetical cell to reflect intracellular, membrane-bound, or extracellular localization. The thickness of a solid line or arrow connecting a given gene pair is proportional to the R² value, which ranges from 0.85 (p=1.5E-17) for the EpCAM2/Claudin3 pair, to 0.55 (p=7.2E-06) for the TFF1/S100P pair. Genes present in the 87 gene list described in the text are depicted by filled ovals. Mal2 was not present in the U133A microarray chips, whereas Spint2 and Esx were present in the U133A chips but not present in the 87 gene list. XAG=AGR2.

FIG. 9 shows real-time RT-PCR analysis of metastatic NSCLC tissue. Real-time PCR analyses of control negative MLN obtained from lung transplant patients (n=13, filled diamonds) and from pathology-positive MLN (n=15, open diamonds) were performed using primer pairs for the indicated genes. Cₜ values for each gene were determined from triplicate reactions. ΔCₜ values were obtained by subtracting the mean Cₜ value of β₂-microglobulin from the mean Cₜ values of the cancer-related genes.

FIG. 10 shows real-time RT-PCR analysis of NSCLC tissue embedded in paraffin. Real-time PCR analyses of negative control (n=13; filled diamonds) or pathology-positive (n=15; open triangles) MLN were performed using primer pairs for the indicated genes. ΔCt values were obtained in triplicate reactions by subtracting the mean Cₜ value of β₂-microglobulin from the mean Cₜ value for each respective gene. XAG=AGR2.

FIG. 11 shows treatment of CRL5876 with Esx siRNA. XAG=AGR2.

FIG. 12 shows AGR2 is overexpressed in metastatic lymph nodes. Real-time PCR analyses of metastatic breast cancer axillary lymph nodes (n=70; open triangles; right side of matched data set) and negative control cervical lymph nodes (left side of each matched data set; filled triangles; n=9-48) was performed as described using primer pairs for the indicated genes. Cₜ values for each gene were determined from triplicate reactions. □Cₜ values were obtained by subtracting the mean Cₜ value of β₂-microglobulin from the mean Cₜ value for each respective gene.

FIGS. 13A and 13B show AGR2 is highly correlated with TFF1 in metastatic breast cancer. Correlation coefficients of the indicated genes with AGR2 were determined as described. Coefficients obtained from nodes derived from ER+ primary tumors (n=53) are shown in FIG. 13A. Coefficients obtained from ER− primary tumors (n=17) are shown in FIG. 13B. Genes (TFF1, ER, and PR) with the three highest correlation values in the ER+ dataset (n=17) are depicted by stippled bars.

FIG. 14 shows AGR2 or TFF1 siRNA inhibits invasion of an ER− cell line. MDA453 cells were transfected in triplicate with the indicated siRNA for 48 hours. Following transfection, cells were assayed for their invasion ability. Shown are the mean values of cell invasion for two separate experiments, with respect to a transfection of a controlled scrambled sequence.

FIG. 15 represents the expression values of the indicated genes as determined by real-time RT-PCR measurements. The horizontal line indicates the threshold value for marker positivity as determined by comparison of normal control samples.

### DETAILED DESCRIPTION OF THE INVENTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods or to particular kits, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a genetic marker” includes mixtures of genetic markers, and reference to “a primer pair” includes mixtures of two or more such primer pairs, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not. For example, the phrase “optionally hybridizes” means that hybridization may or may not occur.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

A “subject” is an individual. Thus, the “subject” can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and more preferably, a human. A “subject” is used interchangeably with a “patient.”

“Non-primary or secondary tissue” is tissue in which the cancer did not first arise and to which the cancer metastasized, or spread, from the primary tissue.

Throughout the specification, the disclosed genetic markers are listed as abbreviations. These abbreviated notations are known to persons of skill in the art and are disclosed with their “aliases,” names that are also used in the art to describe the same genes, in Table 16 below.

The present invention provides methods for the detection, determining prognosis, and treatment of various cancers, including micrometastatic and metastatic breast cancer and non-small cell lung cancer (NSCLC). The detection methods comprise assessing the expression level of specific genetic markers in a cell sample suspected of containing cancerous cells from lymph node tissue of a patient and determining if these genetic markers are overexpressed relative to expression of the respective genetic markers in cells of control tissue, for example, lymph node tissue and blood. Overexpression of one or more genetic markers disclosed herein within the cell sample is indicative of the presence of a cancer of interest in that patient. In some embodiments, expression of a genetic marker disclosed herein is assessed in a cell sample obtained from a patient diagnosed with breast cancer or NSCLC, and overexpression of the genetic marker in the cell sample relative to one or more control genes in the cell sample is predictive of the prognosis for that patient and/or predictive of the patient's clinical response to therapeutic intervention with respect to that cancer.

As used herein, “overexpression” means an expression level that is greater than the expression detected in normal, non-cancerous tissue. For example, a gene that is overexpressed may be expressed about 1 standard deviation above normal, or about 2 standard deviations above normal, or about 3 standard deviations above the normal level of expression. Therefore, a gene that is expressed about 3 standard deviations above a normal, control level of expression (as determined in corresponding non-cancerous tissue) is a gene that is overexpressed and therefore associated with and indicates the presence of metastatic cancer. A person of skill would know a normal level of expression of a nucleic acid (genetic marker) in a non-cancerous tissue. Therefore, it is not necessary for practicing the methods disclosed herein to obtain a normal, non-cancerous control cell sample to be tested each time a cell sample suspected of containing cancer cells is examined for overexpression of a nucleic acid (genetic marker).

The term “prognosis” is recognized in the art and encompasses predictions about the likely course of disease or disease progression, particularly with respect to likelihood of disease remission, disease relapse, tumor recurrence, metastasis, and death, A “favorable prognosis” refers to the likelihood that a patient afflicted with cancer, particularly breast cancer or NSCLC, will remain disease-free (i.e., cancer-free). A “poor prognosis” is intended to mean the likelihood of a relapse or recurrence of the underlying cancer or tumor, metastasis, or death.

Of particular interest to the present invention are methods that provide for detection of micrometastatic breast cancer and metastatic and micrometastatic NSCLC. The terms “metastases” and “metastatic cancer” as used herein refer to cancer that has spread from its site of origin to other parts of the body, is often associated with significant disease burden, and is readily detected by standard histopathologic techniques. The terms “micrometastases” and “micrometastatic cancer” as used herein refer to cancer that has spread from its site of origin to other parts of the body, is often of limited disease burden, and cannot be detected by standard histopathologic techniques. “Occult metastases” and “occult cancer” as used herein refer to metastatic disease that is not detectable by standard hematoxylin and eosin (H&E) staining, but is detectable by more sensitive methodologies such as immunohistochemistry or PCR.

Cancer can metastasize through the lymphatic system to regional nodes and then via the blood to secondary sites. In the present invention, expression levels of genes of interest may be measured in a variety of lymph node tissues including, but not limited to, tissue from axillary lymph nodes (ALN), sentinel lymph nodes (SLN), internal mammary lymph nodes, supraclavicular lymph nodes, and mediastinal lymph nodes (MLN). With respect to the detection and prognosis of breast cancer, preferred lymph node tissue includes tissue from ALN, SLN, internal mammary lymph nodes, and supraclavicular lymph nodes. With respect to the detection and prognosis of NSCLC, preferred lymph node tissue includes tissue from MLN.

Of particular interest to the present invention are two genetic markers, anterior gradient 2 (AGR2) and trefoil factor 1 (TFFI), that have been identified as being predictive of the presence of micrometastatic breast cancer and/or metastatic and micrometastatic NSCLC.

AGR2 (NCBI GenelD 10551; also known as hAG-2) is the human homolog of the Xenopus laevis cement gland-specific gene XAG-2 and is located in chromosome 7p21.3 (Dong et al. (2005) Cancer Res. 65:3796-3805; Thompson et al. (1998) Biochem. Biophys. Res. Commun. 251:111-116; Petek et al. (2000) Cytogenet. Cell Genet. 89:141-142).

TFFI (NCBI GenelD 7031; also known as pS2) is located in chromosome 21 q22.3 and encodes a secretory polypeptide involved in the formation of mucus and that is expressed in the regeneration stage of ulcerative and inflammatory gastrointestinal disorders and in some human carcinomas including some breast carcinomas (Mikhatarian et al. (2005) Clin. Cancer Res. 11:3697-3704; Ribieras et al. (1998) Biochim. Biophys. Acta 1378:F61-F77).

The present invention is directed to the discovery that the overexpression of AGR2 or TFFI in pathology negative axillary or sentinel lymph node tissue of a patient is a useful indicator of the presence of breast cancer micrometastases. The present invention is also directed to the discovery that overexpression of AGR2 in axillary or sentinel lymph node tissue is a useful indicator of the likelihood of responsivity to hormonal therapy in patients with estrogen-receptor-positive and progesterone-receptor-positive (ER+/PgR+) primary breast tumor biopsies. The present invention is further directed to the discovery that the overexpression of AGR2 in mediastinal lymph node tissue of a patient is a useful indicator of the presence of NSCLC metastases or micrometastases. Finally, the present invention is also directed to the discovery that overexpression of AGR2 in a cell sample containing primary, metastatic, or micrometastatic NSCLC cells from a patient diagnosed with early stage NSCLC can be used as a useful indicator of decreased probability of survival. These findings individually provide methods for detecting micrometastatic breast cancer and metastatic or micrometastatic NSCLC with a high degree of specificity and sensitivity and significantly improve the staging and tailored treatment of these cancers.

Thus, in one embodiment, the methods of the invention are directed to detection of micrometastatic breast cancer in a patient. The methods comprise the steps of obtaining a cell sample suspected of containing cancerous cells from lymph node tissue of the patient and determining whether the AGR2 or TFFI genes are overexpressed in the cell sample compared to control lymph node tissue cells, wherein overexpression of AGR2 or TFFI is indicative of the presence of micrometastatic breast cancer in the patient. Lymph node tissue from which cell samples may be obtained include axillary lymph node (ALN) tissue and sentinel lymph node (SLN) tissue. Control lymph node tissue may include cervical lymph node (CLN) tissue. The cell sample may be obtained from tissue that is fixed, paraffin-embedded, fresh, or frozen.

In another embodiment, the present invention provides a method for predicting the likelihood that a breast cancer patient will respond to hormonal therapy. As described more fully in the Experimental Section, in recent years, estrogen receptor (ER) status has emerged as a major prognostic tool for determining the treatment modality for breast cancer patients. ER expression plays an important role in the pathogenesis and maintenance of breast cancer. In breast cancer patients about two-thirds of tumors are ER-positive (Lippman et al. (1980) Cancer 46:2838-2841). Approximately 50% of these ER-positive tumors are estrogen-dependent and respond to endocrine therapy (Mann et al. (1980) Cancer 46:2838-2841).

Thus, in one embodiment of the present invention, the likelihood that a patient diagnosed with breast cancer will respond to hormonal therapy is predicted by determining the expression level of the AGR2 gene in a cell sample containing primary, metastatic, or micrometastatic breast cancer cells obtained from the patient, where a higher expression level of the AGR2 gene compared to the expression level of a control gene within the cell sample is indicative of an increased likelihood of response to treatment with hormonal therapy. In a preferred embodiment, cell samples are obtained from ALN tissue and control genes include, for example, β₂-microglobulin. The comparison of expression levels may be expressed as a ratio of AGR2 gene expression compared to control gene expression. These methods may be carried out using cell samples obtained from tissue that is fixed, paraffin-embedded, fresh, or frozen.

In other embodiments, the methods of the invention are directed to detection of metastatic or micrometastatic NSCLC in a patient. The methods comprise the steps of obtaining a cell sample suspected of containing cancerous cells from lymph node tissue of the patient and determining whether the AGR2 gene is overexpressed in the cell sample compared to control lymph node tissue cells, wherein overexpression of AGR2 is indicative of the presence of metastatic or micrometastatic NSCLC in the patient.

In another embodiment, the present invention provides a method for evaluating prognosis of a patient with NSCLC, particularly the probability of survival for a patient diagnosed with early stage NSCLC. In this manner, a decreased probability of survival for a patient with early stage NSCLC is predicted by determining the expression level of the AGR2 gene in a cell sample containing primary, metastatic, or micrometastatic non-small cell lung cancer cells compared to the expression level of a control gene in that cell sample, where a higher expression level of the AGR2 gene compared to the expression level of the control gene is indicative of a decreased probability of survival. In a preferred method, the determination of the expression level of the AGR2 gene is part of a real-time RT-PCR analysis of a multi-marker panel of genes, particularly where the multi-marker panel of genes includes measurement of expression of the EpCam gene, the PDEF gene, the S1000P gene, or any combination thereof. Preferred control genes include, for example, β₂-microglobulin. Preferred tissues from which cell samples are obtained include mediastinal lymph node (MLN) tissue. Control lymph node tissue may include ALN or MLN from normal patient tissue. The cell sample may be obtained from tissue that is fixed, paraffin-embedded, fresh, or frozen.

In some embodiments described herein, prognostic performance of specific genetic markers and/or other clinical parameters can be assessed utilizing a Cox Proportional Hazards Model Analysis, which is a regression method for survival data that provides an estimate of the hazard ratio and its confidence interval. The Cox model is a well-recognized statistical technique for exploring the relationship between the survival of a patient and particular variables. This statistical method permits estimation of the hazard (i.e., risk) of individuals given their prognostic variables (e.g., overexpression of particular genetic markers, as described herein). Cox model data are commonly presented as Kaplan-Meier curves. The “hazard ratio” is the risk of death at any given time point for patients displaying particular prognostic variables. See generally Spruance et al. (2004) Antimicrob. Agents & Chemo. 48:2787-2792. In particular embodiments, the genetic markers of interest are statistically significant for assessment of the likelihood of decreased probability of survival for a patient diagnosed with early stage NSCLC. Methods for assessing statistical significance are well known in the art and include, for example, using a log-rank test Cox analysis and Kaplan-Meier curves. In some aspects of the invention, a p-value of less than 0.05 constitutes statistical significance.

The methods of the present invention comprise detecting the expression of specific genetic markers in cell samples, for example, cells obtained from a lymph node tissue of a patient in need of diagnostic or prognostic assessment for the cancer of interest. Genetic markers of particular interest include AGR2 and TFF1 depending upon the cancer for which detection or prognostic information is needed. Any methods available in the art for detecting expression of these genetic markers are encompassed herein. The expression of a genetic marker of the invention can be detected on a nucleic acid level or a protein level. By “detecting expression” or “detecting expression level” is intended determining the quantity or presence of a marker gene or protein. Thus, “detecting expression” or “detecting expression level” encompasses instances where a genetic marker is determined not to be expressed, not to be detectably expressed, expressed at a low level, expressed at a normal level, or overexpressed.

In some embodiments, expression level of the genetic marker within a test cell sample, for example, cells obtained from a lymph node tissue of a patient, may be compared with its expression level in a control cell sample, for example, cells that originate from a control lymph node tissue. That is, the “normal” level of expression is the level of expression of the genetic marker in, for example, cells from a control lymph node tissue. By “control lymph node tissue” is intended a lymph node tissue from control subjects without prior history or clinical evidence of malignancy. The control lymph node tissue serves to define baseline expression levels for the genetic marker or markers of interest. In some embodiments, the control lymph node tissue is cervical lymph node or mediastinal lymph node tissue. It is recognized that for some aspects of the invention, no expression, underexpression, or normal expression (i.e., the absence of overexpression) of a genetic marker or combination of genetic markers of interest provides useful information regarding the prognosis of a breast cancer or NSCLC patient.

In other embodiments, expression level of the genetic marker within a test cell sample, for example, cells obtained from a lymph node tissue or a peripheral blood sample of a patient, may be compared with the expression level of one or more control genes within the test cell sample. In this manner, an elevated (i.e., higher) expression level of the genetic marker of interest relative to the expression level of the control gene(s) is predictive of clinical response or likelihood of patient survival.

The detection of genetic markers of the invention includes detection of genes or proteins.

Such genetic markers include DNA comprising the entire or partial sequence of the nucleic acid sequence encoding the marker, or the complement of such a sequence. The marker nucleic acids also include RNA comprising the entire or partial sequence of any of the nucleic acid sequences of interest. A marker protein is a protein encoded by or corresponding to a DNA marker of the invention. A marker protein comprises the entire or partial amino acid sequence of any of the marker proteins or polypeptides. Fragments and variants of marker genes and proteins are also encompassed by the present invention. By “fragment” is intended a portion of the polynucleotide or a portion of the amino acid sequence and hence protein encoded thereby. Polynucleotides that are fragments of a marker nucleotide sequence generally comprise at least 10, 15, 20, 50, 75, 100, 150, 200, 250, 300, 350, 400, 450, 500, 550, 600, 650, 700, 800, 900, 1,000, 1,100, 1,200, 1,300, or 1,400 contiguous nucleotides, or up to the number of nucleotides present in a full-length marker polynucleotide disclosed herein. A fragment of a marker polynucleotide will generally encode at least 15, 25, 30, 50, 100, 150, 200, or 250 contiguous amino acids, or up to the total number of amino acids present in a full-length marker protein of the invention. “Variant” is intended to mean substantially similar sequences. Generally, variants of a particular marker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that marker as determined by sequence alignment programs. In preferred embodiments of the present invention, polynucleotide or polypeptide variants of the AGR2 and TFF1 genes or proteins are measured.

Methods for detecting expression of the genetic markers of the invention comprise any methods that determine the quantity or the presence of the genetic markers either at the nucleic acid or protein level. Such methods are well known in the art and include but are not limited to western blots, northern blots, southern blots, ELISA, immunoprecipitation, immunofluorescence, flow cytometry, immunohistochemistry, nucleic acid hybridization techniques, nucleic acid reverse transcription methods, and nucleic acid amplification methods. In preferred embodiments of the present invention, mRNA transcripts and polypeptide expression products of the AGR2 and TFF1 genes are measured.

In one embodiment, reverse transcriptase reactions coupled to polymerase chain reactions (RT-PCR) are used to assay for the presence of an RNA of interest in a pool of total RNA from a tissue or cell. Detection of a particular RNA is dependent on primers used in the PCR reaction. The initial step in RT-PCR is a reverse transcription step. Procedures for reverse transcription are well known to those skilled in the art, and a variety of procedures can be used. Either total RNA or polyadenylated mRNA can be used as the template for synthesis of cDNA by the reverse transcriptase enzyme. RNA may be isolated from tissue that is fixed, paraffin-embedded, fresh, or frozen (e.g., from frozen or archived paraffin-embedded and formalin-fixed tissue samples that are routinely prepared and preserved in everyday clinical practice).

In one embodiment, oligo(dT) is used as the primer in the reverse transcription reaction. Oligo(dT) hybridizes to the poly(A) tails of mRNAs during first strand cDNA synthesis. Since all mRNAs normally have a poly(A) tail, first strand cDNA is made from all mRNAs present in the reaction (i.e., there is no specificity). In another embodiment, specific primers are used in place of oligo(dT), and specific RNAs are reverse transcribed into DNA. The specific primers preferably are complementary to a region near the 3′ end of the RNA in order that full-length or nearly full-length cDNA is produced. A number of different primers can be used with good results. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.) (see also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York)), Known methods of PCR include, but are not limited to, methods using paired primers, nested primers, single specific primers, degenerate primers, gene-specific primers, vector-specific primers, partially mismatched primers, and the like.

In one embodiment of the invention, primer sequences for amplification of AGR2 include,

(SEQ ID NO:1)

XAG-F1-GCAGAGCAGTTTGTCCTCCTCA
or

(SEQ ID NO:2)

XAG-R1-GGACATACTGGCCATCAGGAGA.

Once the reverse transcriptase reaction is carried out, the cDNA produced is amplified by PCR. The products of the PCR reaction can be detected in various ways. For example agarose gel electrophoresis may be used to separate the DNA in the PCR reaction by size. The agarose gel is then stained with dyes that bind to DNA and fluoresce when illuminated by light of various wavelengths.

The PCR procedure can also be done in such a way that the amount of PCR products can be quantified. Such “quantitative PCR” procedures normally involve comparisons of the amount of PCR product produced in different PCR reactions. A number of such quantitative PCR procedures, and variations thereof, are well known to those skilled in the art. One inherent property of such procedures is the ability to determine relative amounts of a sequence of interest within the template that is amplified in the PCR reaction.

One particularly preferred method of quantitative PCR used to quantify copy numbers of sequences within the PCR template is a modification of the standard RT-PCR called “real-time RT-PCR.” Real-time RT-PCR utilizes a thermal cycler that incorporates a fluorimeter. In one type of real-time RT-PCR, the reaction mixture also contains a reagent whose incorporation into a PCR product can be quantified and whose quantification is indicative of copy number of that sequence in the template. One such reagent is a fluorescent dye, called SYBR Green I (Molecular Probes, Inc.; Eugene, Oreg.) that preferentially binds double-stranded DNA and whose fluorescence is greatly enhanced by binding of double-stranded DNA. When a PCR reaction is performed in the presence of SYBR Green I, resulting DNA products bind SYBR Green I and fluoresce. The fluorescence is detected and quantified by the fluorimeter. Such technique is particularly useful for quantification of the amount of template in a PCR reaction.

Another variation of real-time RT-PCR is TaqMan® (Applied Biosystems, Foster City, Calif.) PCR. The basis for this method is to continuously measure PCR product accumulation using a dual-labeled fluorogenic oligonucleotide probe called a TaqMan® probe. The “probe” is added to and used in the PCR reaction in addition to the two primers. This probe is composed of a short (e.g., 20-30 bases) oligodeoxynucleotide sequence that hybridizes to one of the strands that are made during the PCR reaction. That is, the oligonucleotide probe sequence is homologous to an internal target sequence present in the PCR amplicon. The probe is labeled or tagged with two different fluorescent dyes. On the 5′ terminus is a “reporter dye,” and on the 3′ terminus is a “quenching dye.” For example, reporter dyes may include 6-carboxy fluorescein (FAM), while quenching dyes may include 6-carboxy tetramethyl-rhodamine (TAMRA). When the probe is intact, energy transfer occurs between the two fluorochromes, and emission from the reporter is quenched by the quencher, resulting in low, background fluorescence. During the extension phase of PCR, the probe is cleaved by the 5′ nuclease activity of Tag polymerase, thereby releasing the reporter from the oligonucleotide-quencher and producing an increase in reporter emission intensity. During the entire amplification process the light emission increases exponentially.

In addition to real-time RT-PCR, other procedures can be used to detect RNA that is transcribed from the gene of interest, including Northern blot and dot blot hybridization.

Other methods for detecting and quantifying overexpression of a gene of interest include measurement of polypeptide expression products of the gene. For example, antibodies may be used that are immunospecific for the polypeptide expression products of the gene of interest. The term “antibody” encompasses monoclonal antibodies, polyclonal antibodies, multispecific antibodies (e.g., bispecific antibodies), and antibody fragments, so long as they exhibit the desired biological activity or specificity. “Antibody fragments” comprise a portion of a full-length antibody, generally the antigen binding or variable region thereof. Interactions between antibodies and a target polypeptide are detected by radiometric, calorimetric, or fluorometric means. Detection of antigen-antibody complexes may be accomplished by addition of a secondary antibody that is coupled to a detectable tag, such as for example, an enzyme, fluorophore, or chromophore. Preferably, the detection method employs an enzyme-linked immunosorbent assay (ELISA), Western immunoblot procedure and/or immunoprecipitation.

The present invention also comprises the use of microarray techniques to identify or confirm differential gene expression. In other words, the expression profile of a number of genes of interest can be measured using microarray technology. In this method, polynucleotide sequences of interest (including cDNAs and oligonucleotides) are plated, or arrayed, on a microchip substrate. The arrayed sequences are then hybridized with specific DNA probes from cells or tissues of interest. In a particularly preferred embodiment, microarray techniques may be utilized to detect differential gene expression in a tissue sample of interest using a panel of genes comprising AGR2 (Liu et al. (2005) Cancer Res, 65:3796-3805) or TFFI (Mikhitarian et al. (2005) Clin. Cancer. Res., 11:3697-3704). Other markers of interest include but are not limited to mam, PIP, CEA, CK19, and PDEF (see, e.g., Mitas et al. (2001) Int. J. Cancer, 93:162-171), as well as EpCam (Mitas et al, (2003) Clin Chem., 49:312-315), CEA6 (Aronow et al. (2001) Physiol. Genomics, 6:105-116), GPX2 (Esworthy et al. (2005) J. Nutr., 135:740-745), S100P (Arumugam et al. (2005) Clin, Cancer Res., 11:5356-5364), EpCAM2 (De Leij et al. (1994) Int. J. Cancer Suppl., 8:60-63), Spint2 (Kobayashi et al. (2003) J. Biol. Chem., 278:7790-7799), Ma12 (Marazuela et al. (2004) J. Histochem. Cytochem., 52:243-252), and Esx (Janssens et al. (2004) Mol. Diagn., 8:107-113). Preferred tissues of interest include, but are not limited to, ALN, SLN, internal mammary lymph nodes, supraclavicular lymph nodes, and MLN.

In one embodiment of the microarray technique, PCR-amplified inserts of cDNA clones are applied to a substrate in a dense array. Preferably at least 10,000 nucleotide sequences are applied to the substrate. The microarrayed genes, immobilized on the microchip, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al. (1996) Proc. Natl. Acad. Sci. USA 93:106-149). Microarray analysis can be performed by commercially available equipment, following manufacturer's protocols, such as by using the Affymetrix GenChip® technology.

By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaC1, 1% SDS (sodium dodecyl sulphate) at 37° C., and a wash in 1× to 2×SSC (20×SSC=3.0 M NaC1/0.3 M trisodium citrate) at 50 to 55° C. Exemplary moderate stringency conditions include hybridization in 40 to 45% formamide, 1.0 M NaC1, 1% SDS at 37° C., and a wash in 0.5× to 1×SSC at 55 to 60° C. Exemplary high stringency conditions include hybridization in 50% formamide, 1 M NaC1, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The detection methods of the present invention, including the real time RT-PCR detection methods using multiple genetic markers, detect clinically significant nodal disease that is missed by standard pathology, and for breast cancer patients, is associated with a significant reduction in recurrence-free survival. The methods disclosed herein can be used alone or combined with assessment of clinical information, conventional prognostic methods, and expression of conventional molecular markers known in the art. In this manner, the disclosed methods may permit the more accurate detection of micrometastatic breast cancer and micrometastatic and metastatic NCSLC, improve staging by nodal pathology, and provide predictive information relevant to therapeutic intervention in breast cancer and prognosis of NSCLC patients.

The methods of the invention permit the superior assessment of breast cancer and NSCLC detection and prognosis in comparison to analysis of other known detection and prognostic indicators, as described in more detail in the Experimental section below. In particular aspects of the invention, the sensitivity and specificity is equal to or greater than that of known cancer diagnostic or prognostic evaluation methods.

Those skilled in the art recognize that diagnostic and prognostic assays can be described in terms of accuracy. The term “accuracy” is intended to mean the total number of results of a given test divided by the number of incorrect results. Incorrect results are a function of error rates present in the assay and include but are not limited to measurement error, user error, reporting error, and the like. Diagnostic and prognostic assays can be further described in terms of false positive and false negative rates. False positive and false negative rates are generated by comparing the results of an assay against a gold standard. By the term “gold standard” is intended a reference standard that is unlikely to be incorrect or has been traditionally used to define the disease, such as pathological analysis for metastatic breast cancer and NSCLC, genetic marker analysis for micrometastatic breast cancer and NSCLC, and estrogen and progesterone receptor expression in tumor cells for predicting likelihood of response of breast cancer patients to treatment with hormonal therapy, as described in more detail in the Experimental section below. ROC curve analysis is the most commonly used method for assessing the accuracy of diagnostic tests (Henderson (1993) Ann. ain. Biochem. 30:521-539; see also Experiment 4, Tables 6-8 below). False positive and false negative rates affect the sensitivity and specificity of an assay.

The sensitivity of a test is the probability that it will produce a true positive result when used on a diseased population (as compared to a reference or “gold standard”). The sensitivity of a diagnostic test is calculated as: (the number of true positive results)/(the number of true positive results+the number of false negative results). The specificity of a test is the probability that a test will produce a true negative result when used on a non-diseased population (as determined by a reference or “gold standard”). The specificity of a test is calculated as: (the number of true negative results)/(the number of true negative results+the number of false positive results). The sensitivity and specificity of a diagnostic test indicates possible uses within a particular population. For example, high sensitivity tests are useful in screening populations where the disease to be diagnosed is relatively serious and the treatment is relatively inexpensive and readily available because the cost of a failing to detect a diseased patient is high (false negative) and the cost of treating an undiseased patient is low (false positive). Alternatively, high specificity tests are useful in screening populations where the disease is not as serious and the treatment is relatively expensive because the few undiagnosed, diseased patients (false negatives) within the population will not suffer greatly as compared to the unnecessary treatment of many non-diseased patients (false positives). It is routine within the art to adjust the specificity and sensitivity of assays or use variant assays with differing sensitivity and specificity to screen specific populations. The sensitivity of the disclosed methods for the detection or prognosis of micrometastatic breast cancer, metastatic or micrometastatic NSCLC, or likelihood of response of breast cancer patients to treatment with hormonal therapy is at least about 40%, 45%, 50%, 55%, 60%, 65%, 70%, 75%, 80%, 85%, 90%, 91%, 92%, 93%, 94%, 95%, 96%, 97%, 98%, 99% or more. Furthermore, the specificity of the present methods is preferably at least about 40%, 45%, 50%, 55%, 60%, 65%, 70%, 75%, 80%, 85%, 90%, 91%, 92%, 93%, 94%, 95%, 96%, 97%, 98%, 99% or more, depending upon the diagnostic or prognostic method used.

In further embodiments, the combined sensitivity and specificity value for the diagnostic or prognostic methods of the invention is assessed. By “combined sensitivity and specificity value” is intended the sum of the individual specificity and sensitivity values, as defined herein above. The combined sensitivity and specificity value of the present methods is preferably at least about 105%, 110%, 115%, 120%, 130%, 140%, 150%, 160% or more, depending upon the diagnostic or prognostic method used. In some embodiments, the sensitivity and/or specificity of a panel of markers for the detection or prognosis of micrometastatic breast cancer, metastatic or micrometastatic NSCLC, or likelihood of response of breast cancer patients to treatment with hormonal therapy, is increased by the addition of AGR2 or TFF1 or a combination thereof to the panel of markers.

In another embodiment of the invention, kits are provided that are drawn to reagents that can be used in practicing the methods disclosed herein. The kits can include any reagent or combination of reagents discussed herein or that would be understood to be required or beneficial in the practice of the disclosed methods. For example, the kits could include primers to perform the amplification reactions described, as well as the buffers and enzymes required to use the primers as intended. For example, disclosed is a kit for assessing a subject's risk for cancer metastasis, comprising any one or more of the primer sequences set forth in SEQ ID NOs:1 and 2 and SEQ ID NOs:3-52, as disclosed herein. The kit may also further comprise at least one PCR primer for the amplification of TIFF1, EpCam, PDEF, or S100P, or any combination thereof. The kit can include instructions for using the reagents described in the methods disclosed herein, including but not limited to, methods for detecting micrometastatic breast cancer, methods for predicting the likelihood that a patient diagnosed with breast cancer will respond to hormonal therapy, methods for detecting metastatic non-small cell lung cancer or micrometastatic non-small cell lung cancer, or methods for predicting decreased probability of survival in a patient diagnosed with early-stage non-small cell lung cancer.

In another embodiment of the present invention, methods are also provided for the treatment of breast and non-small cell lung cancer. In particular, the growth of breast cancer cells or non-small cell lung cancer cells in human tissue is inhibited by contacting the tissue with an inhibitor that interacts with a protein, DNA sequence, or RNA sequence of interest, including AGR2 protein, AGR2 DNA, or AGR2 RNA. Such inhibitors include small interfering RNAs (siRNAs), microRNAs (miRNAs), and antisense nucleic acids, and immunotherapies. For example, immunotherapies include the use of antibodies to the AGR2 protein, including monoclonal antibodies, polyclonal antibodies, multispecific antibodies (e.g., bispecific antibodies), and antibody fragments, so long as they exhibit the desired biological activity or specificity. Such inhibitors also include small molecules (molecular weight below about 500 Daltons), large molecules (molecular weight above about 500 Daltons), and polypeptides which compete with a native form of the AGR2 protein for binding to a protein that naturally interacts with the AGR2 protein.

Accordingly, methods of the present invention for inhibiting the growth of breast cancer cells or non-small cell lung cancer cells in human tissue include contacting the tissue with one or more inhibitors of AGR2 function, including, for example, siRNA, miRNA, antisense RNA, and antisense DNA that interfere with AGR2 gene expression, or antagonists of the AGR2 protein, such as anti-AGR2 antibodies.

Methods are further provided for identifying a marker for the detection of micrometastatic disease using a dilutional microarray approach. As described more fully in the Experimental Section and in Mikhitarian et al. (2005) Clin. Cancer. Res., 11:3697-3704, herein incorporated by reference in its entirety, the identification of informative markers for the detection of micrometastatic disease can be simplified by dilution of metastatic tissue (or RNA) into an excess of normal tissue (or RNA). In one method, markers indicative of the presence of micrometastatic disease in a patient are identified involving the steps of: 1) selecting a plurality of candidate markers; 2) diluting a sample of RNA isolated from metastatic tissue into an excess of RNA isolated from non-metastatic tissue at a ratio of at least 1:50 to create a dilution sample 3) measuring the expression levels of the plurality of candidate markers in a set of samples including the dilution sample, an undiluted sample of RNA isolated from metastatic tissue, and a sample of RNA isolated from non-metastatic tissue; and 4) selecting a sub-set of markers from the plurality of candidate markers in which an absence of expression was observed in the sample of RNA isolated from non-metastatic tissue, a fluorescence signal above 500 relative units was observed in the undiluted sample of RNA isolated from metastatic tissue, and a fluorescence signal was observed in the dilution sample; where overexpression of at least one of the markers in the selected sub-set of markers is indicative of the presence of micrometastatic disease in the patient. Micrometastatic diseases for which markers may be identified using these methods include, but are not limited to, micrometastatic breast cancer and micrometastatic non-small cell lung cancer.

Methods are also provided for detecting metastatic cancer in a patient by obtaining a cell sample suspected of containing cancerous cells from lymph node tissue and determining whether one or more genes in a multi-marker panel of genes are overexpressed in said cell sample compared to expression of said genes in control lymph node tissue cells, wherein overexpression of said genes is indicative of the presence of metastatic cancer in said patient, wherein said metastatic cancer is metastatic breast, lung, or pancreatic cancer. In one embodiment, the multi-marker panel of genes comprises the Esx gene. In another embodiment, the multi-marker panel of genes comprises the EpCAM1 gene, AGR2 gene, CKI 9 gene, or CK8 gene, or any combination thereof. In another embodiment, the multi-marker panel of genes comprises the Esx gene, the Map7 gene, the S100P gene, the AGR2 gene, the CEA 6 gene, the GPX2 gene, the TFF1 gene, the Ma12 gene, the Spint2 gene, the EpCAMI gene, the EpCAM2 gene, the CK8 gene, the CK19 gene, or the Claudin3 gene, or any combination thereof. In another embodiment, the multi-marker panel comprises the EpCAM1 gene, the EpCAM2 gene, the AGR2 gene, the Esx gene, the CK19 gene, the CK8 gene, the CEA6 gene, or the Mal2 gene, or any combination thereof, in conjunction with a method for detecting metastatic non-small cell lung cancer. In another embodiment, the multi-marker panel of genes comprises the AGR2 gene, the S100P gene, the CK19 gene, the NQ01 gene, the MET gene, the MAGE-A6 gene, the XAGE-I gene, the KRTHBI gene, the MAGE-A3 gene, or the MAP7 gene, or any combination thereof, in conjunction with a method for detecting metastatic non-small cell lung cancer. In another embodiment, the multi-marker panel of genes comprises the AGR2 gene, the S100P gene, the CK19 gene, the NQ01 gene, the MET gene, the MAGE-A6 gene, the XAGE-1 gene, the KRTI1B1 gene, the MAGE-A3 gene, or the MAP7 gene, or any combination thereof, in conjunction with a method for detecting metastatic non-small cell lung cancer. In another embodiment, the multi-marker panel of genes comprises the AGR2 gene, the S100P gene, the CK19 gene, the Mucinl gene, the FXYD gene, the Claudin3 gene, the CEA6 gene, the GPCR5A gene, the CK7 related gene, or the SCNNIA gene, or any combination thereof, or any combination thereof, in conjunction with a method for detecting metastatic breast cancer. In another embodiment, the multi-marker panel of genes comprises the PNLIPRP2 gene, the CK19 gene, the AGR2 gene, the FXYD gene, the SGP28 gene, the CEA6 gene, the gene of Accession Number AB020676, the Mucinl gene, the gene of Accession Number AB028949, or the MMP19 gene, or any combination thereof, in conjunction with a method for detecting metastatic pancreatic cancer.

Further provided is a method for detecting metastatic cancer in a patient, comprising obtaining a cell sample suspected of containing cancerous cells from lymph node tissue of said patient and determining whether the Esx gene is overexpressed in said cell sample compared to Esx gene expression in control lymph node tissue cells, wherein overexpression of the Esx gene is indicative of the presence of metastatic cancer in said patient, wherein said metastatic cancer is metastatic breast, lung, or pancreatic cancer.

Methods are also provided for the treatment of metastatic breast, lung, or pancreatic cancer comprising inhibiting the growth of metastatic cancer cells in human tissue by contacting the tissue with an inhibitor that interacts with Esx protein, Esx DNA, or Esx RNA and thereby inhibits Esx function. In particular, this method may involve inhibitors that include siRNA, miRNA, antisense RNA, antisense DNA, or antagonists of the Esx protein such as anti-Esx antibodies.

Further provided is a method for detecting prostate cancer in a patient, comprising a) obtaining a cell sample suspected of containing prostate cancer cells from a body fluid of the patient; and b) determining whether the EpCAM2 gene is overexpressed in the cell sample compared to a normal, control level of expression of the EpCAM2 gene expression in a corresponding body fluid sample, wherein overexpression of the EpCAM2 gene in the cell sample is indicative of the presence of prostate cancer in the patient. In one aspect, the body fluid can be blood, plasma, serum, or urine. In one aspect, the patient can have advanced prostate cancer. In another aspect, the patient can have clinically undetectable prostate cancer.

### EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### EXAMPLES

### RNA Isolation and Real Time RT-PCR Methods for Examples 1-6

For use in the following examples, RNA isolation may be carried out using either a modified guanidinium method or the Specht method.

Modified Guanidinium Method: Total cellular RNA is isolated from 50-micron (μ) sections from each paraffin embedded sample. Sections are deparaffinized by incubation in xylene at 37° C. twice. After deparaffinization, samples are washed with ethanol and allowed to air dry. The dried pellet is resuspended in 200 of 6 mg/ml proteinase K, 1 M guanidinium thiocyanate, 25 mM 2-mercaptoethanol, 0.5% Sarkosyl™, 20 mM Tris-HCl, pH 7.5, and incubated for several hours at 45° C. The sample is then extracted, using an equal volume, with phenol:chloroform:isoamyl:alcohol (25:24:1) and the non-organic aqueous layer transferred to a clean, RNase-free tube. The RNA is precipitated, using 2 pg glycogen as carrier, with isopropanol and an incubation at −80° C. After a 30 minute centrifugation, the RNA pellet is washed with 70% ethanol, and allowed to air-dry. The sample is resuspended in RNase-free water and stored −20° C.

Specht Method (Specht et al. (2001) Am. J. Pathol. 158:419-429): Total cellular RNA is isolated from two 10-micron sections from each paraffin embedded sample. Sections are deparaffinized by incubation in xylene at room temperature twice. After deparaffinization, samples are washed with ethanol (100%, 90%, then 70%) and allowed to air dry. The dried pellet is resuspended in 200 p. 1 of 500 pg/m1 proteinase K, 10 mM Tris-HC1, pH 8.0, 0.1 mM EDTA, pH 8.0, 2% SDS, pH 7.3, and incubated for 16 hours at 60° C. The sample is then extracted, using an equal volume of phenol:chloroform:isoamyl alcohol (25:24:1) and the non-organic aqueous layer transferred to a clean, RNase-free tube. The RNA is precipitated, using 0.1 volume 3 M Sodium Acetate, 10 jig glycogen as carrier, 1 volume isopropanol, and an incubation at −20° C. overnight. After a 30 minute centrifugation, the RNA pellet is washed with 70% ethanol, and allowed to air-dry. The sample is resuspended in 10 pl RNase-free water and stored at −20° C.

Real-time RT-PCR: Real-time RT-PCR is performed on a PE Biosystems Gene Amp®) 7000, 7300, or 7500 Sequence Detection System (Foster City, Calif.). All reaction components are purchased from the same supplier. Reactions are performed as described by Mitas and colleagues (Mitas et al. (2001) Int. J. Cancer 93:162-171), using gene specific primers for the RT.

Sensitivities of Taqman and SYBR Green Chemistries: Previous studies by Schmittgen and colleagues have shown that although the dynamic range and sensitivity of SYBR green is comparable to TaqMan, SYBR green detection is more precise and produces a more linear decay plot (Schmittgen et al. (2000) Anal. Biochem. 285:194-204). These results were confirmed in studies comparing sensitivities of the two chemistries using representative and informative cancer-associated genes. Real-time RT-PCR using SYBR Green or Taqman chemistries were performed with mam primers and 10-fold serial dilutions of eDNA prepared from the breast cancer cell line MDA361. In addition, real-time experiments were also performed using synthetic fragments to EpCam and lunx amplicons. All dilutions were split into separate fractions; one for SYBR Green analysis, and the other for TaqMan analysis. All reactions were performed in triplicate. Comparable sensitivities of gene detection using SYBR green and Taqman were observed. For the three genes using Taqman analysis, the mean correlation coefficient value was 0.992+0.004, a value below that of SYBR Green (0.999±0.001). This result indicates that signals generated from SYBR Green are more precise compared to Taqman. Therefore, for real time RT-PCR methods described in Examples 1-5, SYBR Green chemistries are used.

### Example 1

### Identification of Markers for Detection of Micrometastatic Disease

Rationale: The purposes of this study were to determine why some molecular markers are better than others in previous studies for detection of micrometastatic disease and to use this information to search for additional markers.

Design: Frequency distributions of gene expression values previously obtained as described above (Gillanders et al. (2004) Ann. Surg. 239:828-840) were generated and analyzed using a MATLAB 6 programming environment. Next, a microarray analysis was performed using a metastatic axillary lymph node in which mam was overexpressed at a level 5.3×10⁷-fold higher than the mean expression in normal lymph nodes. RNA from the metastatic lymph node was diluted into a pool of normal lymph node RNA at ratios of 1:50, 1:2,500 and 1:125,000. For all of these conditions, expression values were obtained for a total of 22,283 gene transcripts spotted on an Affymetrix U133A array. Finally, the microarray analyses were repeated using two metastatic tissues in which mam was expressed at low levels compared to other cancer-related genes.

Results: For all seven molecular markers (mam, mamB, muc 1, CEA, PDEF, CK19, and PIP), the distribution of gene expression in the H&E (+) ALN was bimodal (Gillanders et al. (2004) Ann. Surg. 239:828-840). Based upon this distribution, it was concluded that the population expressing a given marker at high levels corresponds to ALN tissues containing metastatic breast cancer. Mean values for metastatic and control normal populations were then determined, thus allowing for calculation of relative levels of gene expression (RLGE) for all seven markers (see Livak and Schmittgen (2001) Methods 25:402-408). The matn marker had the highest RLGE value (1.9×10⁶), whereas muc 1 had the lowest (3.6×10²).

To determine whether RLGE values were correlated with the ability of a given marker to detect micrometastatic disease, a linear regression analysis was performed. It was observed that the correlation coefficient between log [RLGE] values and detection of micrometastatic disease in the H&E (−) population was good (R2=0.69, p=0.0211 [F-test]). This result provides statistical validation of the concept that the most informative markers for detection of micrometastatic disease are those that are most highly overexpressed in metastatic disease.

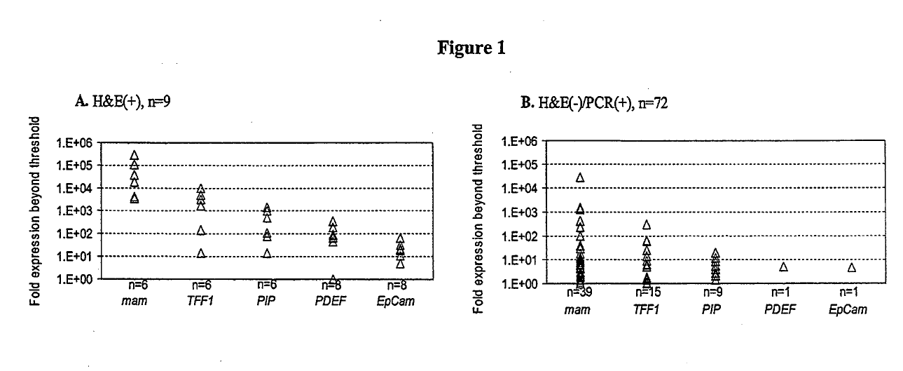

To test this hypothesis, an innovative microarray strategy was developed (see Example 6 and Mikhitarian et al. (2005) Clin. Cancer. Res., 11:3697-3704, incorporated by reference herein in its entirety). Briefly, RNA from an ALN containing metastatic breast cancer was diluted into RNA from a normal lymph node, and analyzed using Affymetrix microarrays. Expression analysis indicated that only two genes (mammaglobin [mam] and trefoil factor I [TFF1]) were significantly overexpressed at a dilution of 1:50. Real-time RT-PCR analysis of FFPE H&E+ALN (n=9) and fresh-frozen H&E−ALN (n=72) confirmed that of all the markers tested, mam and TFF1 had the highest apparent sensitivity for detection of micrometastatic breast cancer (FIG. 1; see also Example 6 and Mikhitarian et al. (2005) Clin. Cancer. Res., 11:3697-3704). Based on these results, it was concluded that a dilutional microarray approach is a simple and reliable method for the identification of informative molecular markers for the detection of micrometastatic cancer.

In microarray analyses using two metastatic tissues in which mam was expressed at low levels compared to other cancer-related genes, genes were selected based on the absence of expression in normal lymph node tissue and presence of expression in the 1:50 metastatic tissue dilutions. Expression values of the 1:50 dilutions were combined and divided by the value obtained for the normal lymph node tissue, thus yielding a ratio value for each gene. Genes were sorted according to numeric ratio values, the top 5 of which are listed in Table 1. Of the top 5 genes, only two (CK19 and TFFI) have been previously reported to be associated with breast cancer. Of the remaining 3 genes, expression levels for only 2 (AGR2 and carboxypeptidase B1 (CPB1)) were higher than 200 fluorescent units.

TABLE 1
Candidate breast cancer genes identified from metastatic tissues
that express mammaglobin at relatively low levels.

| Rank | Gene | Met A | Met B | Total | Normal | Ratio |
|---|---|---|---|---|---|---|
| 1 | CK19 | 106 | 323 | 430 | 2 | 287 |
| 2 | AGR2 | 72 | 409 | 480 | 3 | 185 |
| 3 | Cartilage | 6 | 49 | 55 | 1 | 42 |
| | oligomeric | | | | | |
| | matrix protein | | | | | |
| 4 | Carboxypeptidase | 674 | 28 | 703 | 19 | 38 |
| | BI | | | | | |
| 5 | Trefoil | 498 | 52 | 549 | 17 | 33 |
| | factor 1 | | | | | |
| 12 | EpCam | 26 | 46 | 72 | 8 | 9 |
| 14 | v-fos FBJ | 40 | 29 | 69 | 7 | 9 |
| | osteosarcoma | | | | | |
| | oncogene | | | | | |

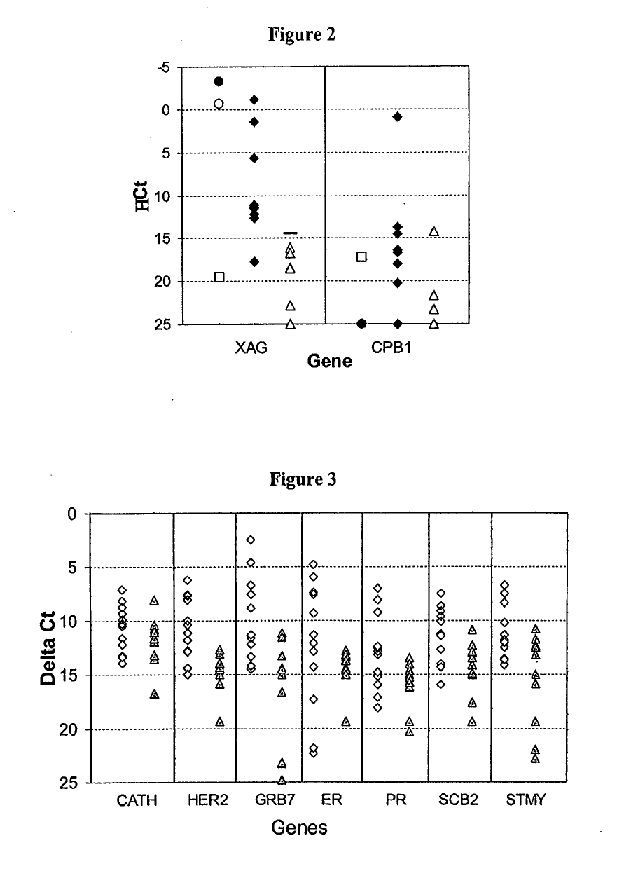

To investigate whether either AGR2 or CPB1 were informative diagnostic markers, their levels of expression were determined in nine pathology positive LN and in nine cervical control lymph nodes. It was observed that AGR2 was overexpressed in 8/9 (89%) LN (FIG. 2), demonstrating that AGR2 is an informative marker of metastatic disease. Interestingly, AGR2 was also expressed at high levels in MDA361 and MDA453, estrogen receptor (ER) positive cell lines. In contrast, expression was not detected in MDA231, an ER-negative cell line. This result is consistent with previous studies showing that AGR2 is coexpressed with the ER in breast cancer cell lines (Thompson and Weigel (1998) Biochem. Biophys. Res. Commun. 251; 111-116; Fletcher et al. (2003) Br. J. Cancer 88:579-585). Interestingly, a recent study has shown that the gene encoded by AGR2 acts as a cell survival factor by inhibiting the function of p53 (Pohler et al. (2004) Mol. Cell. Proteomics 3:534-547).

Because important information regarding cancer progression can be obtained by identifying groups of genes that are co-regulated, the Cancer Genome Anatomy Project (CGAP, National Cancer Institute, Bethesda, Md.) was queried for genes whose expression was most highly correlated with TFF1 in 60 cancer cell lines (NCI60). Among the genes/sequences identified that had a positive correlation P value <1×10⁻⁵ was AGR2, providing evidence that in many cell types, a common mechanism regulates expression levels of TFF1 and AGR2.

The aforementioned results indicate that AGR2 is an informative gene for detection of micrometastatic disease, and that the addition of TFF1 and AGR2 to multi-marker real-time RT-PCR panels will significantly increase prognostic value of such assays.

### Example 2

### Identification of Genes Predictive for Responsivity to Hormonal Therapy

Genes that are predictive for response to tamoxifen therapy have been previously identified in the GH/NSABP study (Paik et al. (2004) N. Engl. J. Med. 351:2817-2826). The utility of these genes in the lymph node setting is not known and largely depends upon the degree to which these genes are overexpressed compared to normal lymph node. This study is designed to identify a gene expression signature profile that correlates with favorable outcome using FFPE SLN from ER+/node+, tamoxifen-treated patients, including those with recurrent disease and those that have been disease-free at least 5 years post-surgery. Disease-free and recurrent patient groups are matched for tumor size/stage at diagnosis. SLN are subjected to real-time RT-PCR analysis using a carefully selected 14-gene marker panel that includes TFF1, AGR2, five genes that are diagnostic for detection of metastatic disease, as well as six prognostic genes known to be associated with disease recurrence in ER+/node− patients treated with tamoxifen (Paik et al. (2004) N. Engl. J. Med. 351:2817-2826). Reliable reference control genes are also identified. Expression values are analyzed, and an algorithm that predicts disease recurrence is developed.

Identification of control genes: The “gold-standard” normalization gene is still the subject of debate. In cancer research, only a few studies have attempted to investigate the variation in expression of housekeeping genes between tissue samples (Degan et al. (2000) Diagn. Mol. Pathol. 9:98-109; Gerard et al. (2000) Mol. Diagn. 5:39-46; Goidin et al. (2001) Anal. Biochem. 295:17-21). Mostly, only two or three candidate genes were compared in these studies. Although more recent studies have analyzed expression of 12-13 reference control genes (e.g., Lee et al. (2002) Genome Res. 12:292-297; de Kok et al. (2005) Lab Invest. 85:154-159), these studies did not analyze lymph node, the tissue used in the present study. The use of a reliable endogenous control gene is critical for accurate diagnosis of metastatic disease. For this reason, reference genes that are suitable for molecular analysis of lymph node tissue must be identified.

A total of 10 widely used housekeeping genes are initially tested (Table 2). All genes are constitutively expressed in various tissues. Further, all genes have independent functions in cellular maintenance, and regulation of their expression is assumed not to be related directly (de Kok et al. (2005) Lab. Invest. 85:154-159). Tissue samples for this portion of the study include H&E− FFPE ALN.

TABLE 2
Internal reference control genes

| Gene | | | |
|---|---|---|---|
| # | Abbr. | Gene name | Cellular function |
| 1. | BACT | β-actin | Cytoskeleton |
| 2. | CYC | Cyclopilin A | Serine Theonine |
| 3. | GAPDH | Glyceraldehyde-3- | Serine theonine |
| | | phosphate dehyd. | phosphatase-inhibitor |
| 4. | PGK | Phosphoglycerokinase 1 | Glycolysis enzyme |
| 5. | B2M | β-microglobulin | Major histocompatibility |
| | | | complex |
| 6. | BGUS | β-glucoronidase | Exoglycosidase in |
| | | | lysosomes |
| 7. | HPRT | Hypoxanthine rib | Metabolic salvage |
| | | osyltransferase | of purines |
| 8. | TBP | TATA-box binding | Transcription by RNA |
| | | protein | polymerases |
| 9. | PBGD | Prophobilinogen | Heme synthesis |
| | | deaminase | |
| 10. | TfR | Transferrin receptor | Cellular iron uptake |

Data analysis for this portion of the study includes Principal Component Analysis (PCA) with the help of SAS statistical software to find the set of linear combination of genes capturing maximum variability of the observed gene-expressions. The assumptions of the analysis are: 1) all measurement errors of individual gene-expressions are Gaussian; 2) a linear dependency among genes is able to explain the variability of gene-expressions effectively; and 3) multivariate Gaussian (Normal) distribution of the genes is within a subject/unit. PCA allows clustering of genes based on various linear patterns; genes clustering outside the diagonal of the correlation matrix are excluded from the set of reference control genes. For the entire reference set, to investigate how much the expression of a single gene contributes to (or is correlated with) the mean expressions of the genes, simple linear regression methods (in SAS) are used and the R-square statistic is computed (this gives the % of variability in mean expressions explained by the individual gene's expression).

To compensate for variations in RNA degradation, a separate analysis is performed using normalized gene expression values. Normalization is performed similar to that used for Affymetrix microarray studies in the following manner. First, the mean Cₜ value for all genes in the H&E−ALN set is determined (=MCon). Second the mean Cₜ value of genes for a given lymph node (x) is determined (=MLN(X)). Third, for LN(x), the difference between MCon and MLN(X) is calculated (=ΔM(x)). Fourth, ΔM(x) is subtracted (or added) to expression values derived from LN(x) (=normalized expression values). This process is repeated for the entire H&E− data set. Normalized expression values are subjected to PCA and linear regression as described above.

For selection of reference control genes, if the standard deviation value of expression for a given gene is relatively low, it is assumed that the gene is potentially useful as an internal reference control. The minimum number of genes whose mean level of expression is within 90% of the total gene set 95% of the time is selected.

Establishment of positive controls using FFPE tissue culture cells: For a clinical diagnostic test, adequate controls are necessary for thorough evaluation of real-time RT-PCR data. Positive controls are especially important for ruling out the possibility of false negative results and are also necessary to ensure that results generated from one lab can be compared to another. Positive controls are also necessary for establishing the precision of an assay. According to the FDA, precision is defined as the variability in the data from replicate determinations of the same homogeneous sample under the normal assay conditions (FDA (1995) FDA International Conference on Harmonization, vol. Docket No. 94D-0016). For enzyme assays, acceptable variability is usually <10%, while variability for in vivo and cell based assays is usually 20 to 50%. Variability for virus titer assays is usually >300%. Precision includes within assay variability, repeatability (within-day variability), and reproducibility (day-to-day variability). Precision may be established without the availability of a “gold” standard as it represents the scatter of the data rather than the exactness (accuracy) of the reported result.

For purposes of defining acceptable precision for a diagnostic test for micrometastatic disease detection, the test likely falls into an enzyme-based assay. Thus, the variability of the assay should be within 10%. The goal of this study is the development of an assay with a precision of 90%. Since a conservative value for marker positivity in the present real-time RT-PCR measurements is a ACₜ value of 15, a 10% variability would equate to 1.5 ΔCt units. To satisfy the requirement that positive control samples be homogeneous, tissue culture cells embedded in paraffin are used.

Briefly, 1×10⁷ tissue culture cells are pelleted and resuspended in buffer containing 10% formalin. An equal volume of liquefied HistoGel (Richard-Allan.Scientific, Kalamazoo, Mich.) is added at 50° C. and vortexed. The solidified gel is then processed as a standard histology specimen (without wrapping in lens paper) and embedded in paraffin. Sections are then cut.

Selection of a diagnostic gene panel for FFPE tissue analysis: The selection of diagnostic genes is based on extensive studies whereby approximately 270 primer pairs to 130 genes are screened using control negative and control positive lymph nodes. Sources for these genes included previous PCR studies described in the literature, the Cancer Genome Anatomy Project (CGAP) SAGE and cDNA library database, genes reported in the literature to be upregulated in various cancers, and finally, genes identified in dilutional microarray analyses described in Example 1 above (Mikhitarian et al. (2005) Clin. Cancer Res. 11:3697-3704). The primary criterion used to select genes in the diagnostic category was overexpression in metastatic tissue at a level approximately 1000-fold higher compared to normal tissue. Genes that met this criterion were mam, PIP, CEA, CK19, PDEF, EpCam, TFF1, and AGR2. The gene mamB was excluded from the list since it was diagnostically redundant to mam.

Genes that are predictive for response to tamoxifen therapy have been previously identified in the GH/NSABP study (Paik et al. (2004) N. Engl. J. Med. 351:2817-2826). The utility of these genes in the lymph node setting is not known and largely depends upon the degree to which these genes are overexpressed compared to normal lymph node. To determine which genes are expressed to a sufficient level to be used for this purpose, primers to the 16 genes used in the GH study were designed and validated using cDNA prepared from a breast cancer cell line in the absence or presence of reverse transcriptase. The expression of the 16 genes in four normal lymph nodes and in four metastatic lymph nodes was measured. Mean levels of expression of only four genes were at least 10-fold higher in metastatic tissue compared to normal tissue (Table 3; ratio determined as mean level of expression in metastatic tissue (n=4)/mean level of expression in normal lymph node (n=4)). Two of these genes (ER and PR) were in the ER family of genes, while two (GRB7 and Her2) were in the Her2 family of genes.

TABLE 3
Genes used in GH study are modestly overexpressed
in metastatic lymph node tissue

| Rank | Gene | Ratio |
|---|---|---|
| 1 | ER | 67.1 |
| 2 | GRB7 | 62.7 |
| 3 | Her2 | 44.4 |
| 4 | PR | 38.7 |
| 5 | Scb2 | 6.3 |
| 6 | CatL | 4.8 |
| 7 | STKI 5 | 3.3 |
| 8 | Bcl2 | 3.1 |
| 9 | STMY | 2.9 |
| 10 | CD68 | 2.0 |
| 11 | GSTMI | 2.0 |
| 12 | MYBL2 | 1.5 |
| 13 | CycB 1 | 1.1 |
| 14 | KI-67 | 0.9 |
| 15 | BAGI | 0.6 |
| 16 | SRV1 | 0.3 |

To further investigate the potential use of the candidate genes, the expression levels of the six most promising genes were measured in a total of 12 metastatic tissues and in 10 normal tissues. High expression of Her2, GRB7, ER, and PR was observed in several tissues (FIG. 3).

To compare the value of the GH genes for detection of micrometastatic disease to genes previously identified in Example 1, mean expression values of metastatic populations were needed. Mean expression values of a metastatic population can be estimated by calculating the mean of the highest 40% expression values. Using the ΔCₜ method as described in Example 1, the fold-overexpression values for the GH genes were calculated, which are listed in ˜rank order along with those used in Example 1 (Table 4; D=prognostic gene, P=prognostic gene, D/P=diagnostic and potentially prognostic gene). Genes listed in Table 4 constitute the gene panel for the FFPE tissue study, as compared to a reference control gene as described above. GH genes were GRB7, ER, Her2, STMY, PR, and SCB2. Values of fold-overexpression listed in Table 4 indicate that the ability of the GH genes to detect micrometastatic disease is relatively low.

TABLE 4
Fourteen-marker gene panel to be used for
node-positive and node-negative patients

| Genes Selected | Category | Fold-Overexpression |
|---|---|---|
| Mam | D | 1,890,000 |
| ECU] | D | ND |
| ECU3 | D | ND |
| PDEF | D | 8,600 |
| PIP | D | 140,000 |
| EpCam | D | 9,340 |
| TFF1 | DIP | 159,000 |
| AGR2 | DIP | 16,000 |
| GRB7 | P | 2,000 |
| ER | P | 179 |
| Her2 | P | 112 |
| STMY | P | 102 |
| PR | P | 68 |
| SCB2 | P | 40 |

Measurement of the expression the gene panel in FFPE, ER+/node+ tissues: Tissues used for this study consist of archived FFPE SLN from ER+/node+, tamoxifen-treated patients who have undergone surgical treatment. A portion of the patients are disease-free at least five years post-surgery, while the remaining experience disease recurrence. Disease-free and recurrent patient groups are matched for tumor size/stage at diagnosis. SLN are analyzed by H&E to verify the presence of metastatic disease in a given node. If SLN are not available for a given patient, two axillary lymph nodes that appear to contain the highest metastatic disease content are identified. Samples from each H&E+ node are analyzed by real-time RT-PCR.

For each PCR analysis, two controls are included: a no-template control (NTC), and a positive PCR control (described above).

SYBR Green I chemistry is used for all reactions. The rationale for the use of this chemistry is that primer sets for all genes have been thoroughly tested using SYBR Green. All real-time data are converted to Excel files.

A no template control condition is used to assess the presence or absence of amplification to assess contamination. If amplification is observed in the no template control sample, the patient samples analyzed in the same batch are excluded from the study.

An endogenous RNA control can also be used. The ability to determine levels of gene expression is dependent upon adequate recovery of mRNA and sufficient conversion to cDNA. Eligibility criteria need to be established so that tissue samples containing less than adequate amounts of mRNA are not included in the study. Low sample quality and/or low sample yield may prevent the ability to obtain reliable real-time RT-PCR measurements. For this reason, it is important to use an objective measurement that assesses whether a sample can be included in the study. The most practical measurement for this purpose is the level of endogenous control gene(s). Thus, endogenous RNA controls are used as identified in the above section describing selection by AUC values. Samples for which the β₂-microglobulin Cₜ values are above 21 are excluded.

A positive control template can also be used. Failure to observe amplification of a specific gene can be due to a variety of factors, including failure to isolate RNA of suitable quality, failure to convert RNA to cDNA, or suboptimal PCR conditions. To address these issues, RNA purification and PCR amplification of positive control samples is performed. Positive control samples are processed in parallel for each set of test samples. For positive control, FFPE MDA36I tissue culture cells are used. Failure to amplify a specific gene provides evidence for a problem with a particular primer set. Failure to achieve optimal (or any) amplification of all genes indicates a problem with the master mix and/or cDNA synthesis step. If a problem with gene amplification is uniformly observed, RNA extraction, cDNA synthesis, and real-time PCR are repeated with all test samples and a new control sample.

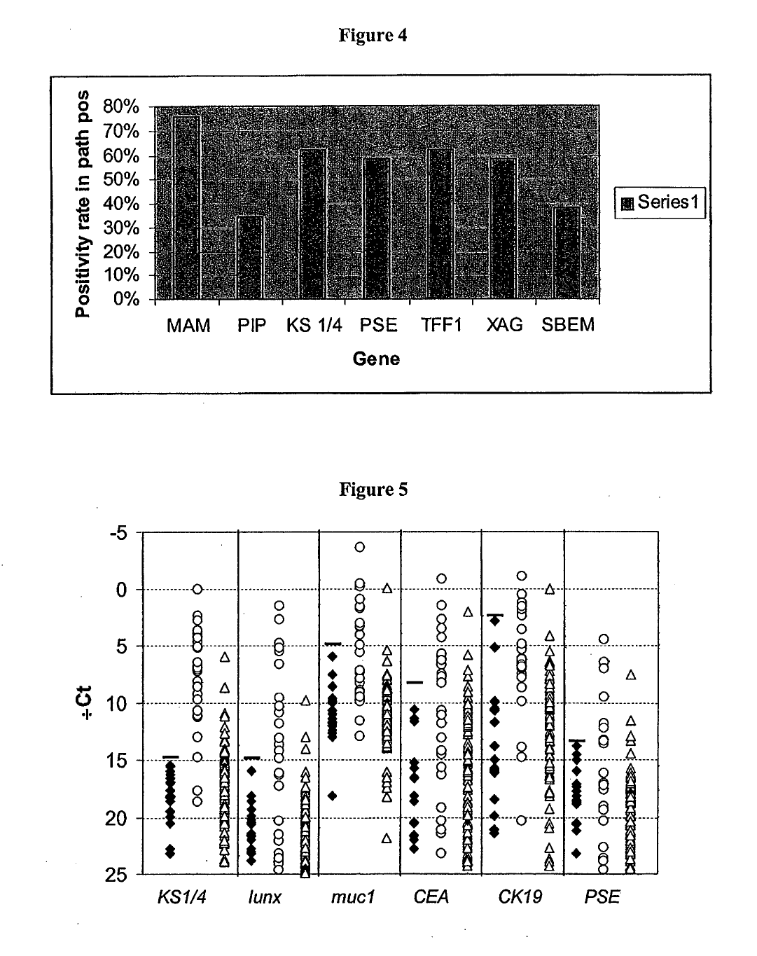

In the present study, 36 axillary lymph nodes were examined by real-time PCR for expression of various cancer-associated genes. Twenty-nine nodes were determined to express at least one gene at significantly elevated levels (FIG. 4). AGR2 was overexpressed in 59% of the nodes. These results indicate that the expression of AGR2 relative to control genes is a prognostic indicator of response to hormonal therapy.

Algorithms for prediction of clinical outcomes from gene expression values: Results may be analyzed using various statistical tools. In the simplest scenario, the algorithm arrives at threshold levels of marker positivity such as that described above. More complicated scenarios involve the weighting of specific markers, such that ΔCₜ values of specific genes are multiplied by various constants and added with other genes.

One approach includes the use of artificial neural networks (ANN). There is an emerging consensus that like other systemic dysfunctions, the molecular basis for cancer is “robust” (Katino (2004) Nat. Rev. Cancer 4:227-235) in the sense that it depends on non-linear interdependencies between components. Consequently, part of the predictive association between gene expression levels and the phenotype depends on the co-variance between, rather than the variance of, individual expressions. Accordingly, discriminant analysis techniques that can accommodate XOR (exclusive OR) interdependencies may be used. Guidelines with regard to early stop procedures and topology optimization that have guided software implementation are known and have been validated by a number of environmental (Nobel et al. (2000) Appl. Environ. Microbiol. 66:694-699), biotechnological (Wolf et al. (2001) Biotechnol. Bioeng. 72:297-306; Wolf et al. (2003) Water Sci. Technol. 47:161-167), biomolecular (Almeida et al. (2005) Proteomics 5:1242-1249; Voit and Almeida (2004) Bioinformatics 20:1670-1681; Almeida and Voit (2004) Genome Inform. Ser. Workshop Genome Inform. 14:114-123), and clinical (Mueller et al. (2003) AMIA Annu. Symp Proc. 945; Mueller et al. (2004) Pediatr. Res. 56:11-18) applications.

Another approach involves a model-free method to identify dependency and dynamic structure that lead to increased efficiency for dimensionality reduction (Garcia and Almeida (2002) Curr. Opin. Biotechnol., 13:72-76). Such techniques have been utilized in the pursuit of closest neighbor evaluation for the identification of optimal combinations of genes in a RT-PCR dataset for discrimination between Barrett's Esophagus and esophageal adenocarcinoma (Mitas et al. (2005) Clin. Cancer Res. 11:2205-2214). Such an approach may involve computational assessment of all possible gene combinations, or more conventional iterative variable selection.

For example, the classification of each new patient is predicted by the classification of the previous patient that yielded the closest gene expression profile. Then the combination of genes is determined that should be used to achieve the best results. Because interdependency between expression of different genes is the exception rather than the rule, genes picked for the best combination of a given number of genes are not always expected to be present in the best combination of a larger number of genes. Such an approach is therefore one of model-free pattern recognition.

Another approach is a full combinatorial, model-based method. For each of the candidate combinations, a discriminant model (most likely an ANN) is used instead of the closest neighbor found with a Euclidean metric, which does not weight for different discriminant value by the candidate genes. This approach allows for analysis of RT-PCR results where the smaller number candidate markers enables full combinatorial search in useful time-frames. The critical feature of this approach is the correction for over-fitting that is achieved by three layers of validation—internal cross-validation nested within external validation for variable selection, itself nested in a final validation with a completely independent dataset. The typical outcome includes a proteomic marker selection example where the optimal number of discriminant biomarkers is found to be two, and also found to follow a XOR interdependency which had caused it to elude more conventional methods that assume linear independency between candidate markers.

Validation of gene panel assay: Validation data consist of archived FFPE SLN from ER+/node+ patients. Data are analyzed to determine whether the algorithm can accurately predict disease recurrence. If the algorithm is able to discriminate between the two outcome groups at a p value <0.05, it is concluded that the assay is of clinical value.

### Example 3

### Identification of AGR2 as a Highly Sensitive Marker to Detect Metastatic Non-Small Cell Lung Cancer (NSCLC)

The present study was to determine whether informative genes of high diagnostic value could be identified using a microarray approach. It was reasoned that genes of high diagnostic accuracy would be highly expressed in at least several NSCLC cell lines with respect to normal lymph node.

Design: RNA was prepared from lung cancer cell lines CRL5809 (bronchioalveolar carcinoma), CRL5876 (adenocarcinoma), A549 (adenocarcinoma), and HTB177 (large cell carcinoma). Gene expression values from each cell line were obtained for a total of 22,283 transcripts spotted on an Affymetrix U133A array. As negative control, expression values of normal lymph node RNA were also determined. Potential diagnostic genes were selected based on the following criteria: 1) absence of expression in normal lymph node; and 2) detectable expression in at least 2 lung cancer cell lines. Genes were then sorted according to their average fluorescence values obtained for the cell lines (from highest to lowest). For the top 20 genes, the ratio of fluorescence signals in cell line to normal lymph node was calculated. Genes were then resorted according to the highest ratio value (Table 5).

Results: As shown in Table 5, the gene with the highest apparent sensitivity for detection of NSCLC was AGR2, a gene recently shown to be associated with metastatic breast cancer (Lie et al. (2005) Cancer Res. 65:3796-805). AGR2 was also recently identified as a gene that was overexpressed by circulating tumor cells in peripheral blood of colon, prostate, breast, and pancreatic cancer patients (Smirniv et al. (2005) Cancer Res. 65:4993-4997). Two melanoma-associated antigen genes (MAGE-3 and MAGE-6) were among the top 4 most highly overexpressed genes. MAGE-3 is a promising target for immunotherapy because it is exclusively presented on the cell surface of cancer cells and might be associated with an aggressive cancer phenotype (Sienel et al. (2004) Eur. J. Cardiothorac. Surg. 25:131-134). Evidence suggests that Immunotherapy using MAGE peptides may be effective for treating at least some NSCLC patients (Sienel et al. (2004) Eur. J. Cardiothorac. Surg. 25:131-134; Morse et al. (2005) J. Transl. Med. 3:9; Atanackovic et al. (2004) J. Immunol. 172:3289-3296). Of the genes used in the previous studies of metastatic NSCLC disease described above (Mitas et al. (2003) Clin. Chem. 49:312-315; Wallace et al. (2005) Chest 127:430-437), only one (EpCam/KS1/4) was present in the top 20 genes identified by microarray analysis. Interestingly, the second (and only other) member of the EpCam gene family (EpCam2) was also identified and predicted to be comparable in sensitivity to EpCam.

TABLE 5
Genes highly overexpressed in NSCLC cell
lines compared to normal lymph nodes

| | | Accession | Negative | Lung | |
|---|---|---|---|---|---|
| Rank | Gene | # | Control | Ca | Ratio |
| 1 | AGR2 | NM_006408 | 2 | 1824 | 960 |
| 2 | S100P | NM_005980 | 3 | 2564 | 754 |
| 3 | MAGE-6 | NM 005363 | 6 | 1897 | 311 |
| 4 | MAGE-3 | NM 005362 | 13 | 2327 | 178 |
| 5 | aldo-keto reductase | NM_020299 | 56 | 5589 | 101 |
| | B 10 | | | | |
| 6 | TACSTD2 (EpCam2) | NM 002353 | 28 | 2623 | 94 |
| 7 | TACSTD1 (EpCam) | NM 002354 | 22 | 1992 | 91 |
| 8 | unknown | AK000345 | 20 | 1377 | 71 |
| 9 | aspartate beta- | AF306765 | 77 | 3871 | 51 |
| | hydroxylase | | | | |
| 10 | interleukin 8 | NM 000584 | 27 | 1281 | 48 |
| 11 | FER1 L3 | NM_013451 | 68 | 2321 | 34 |
| 12 | LC27 | NM_006408 | 51 | 1617 | 32 |
| 13 | preg-sp. beta-1 | M34421 | 66 | 1824 | 28 |
| | glycopro. (PSG) | | | | |
| 14 | UCHL1 | NM 004181 | 102 | 2085 | 20 |
| 15 | unknown | NM_016002 | 68 | 1324 | 19 |
| 16 | dihydrodiol | NM 001354 | 244 | 3312 | 14 |
| | debydrogenase | | | | |
| 17 | PSG4 | NM 002780 | 109 | 1465 | 13 |
| 18 | tumor protein | NM 003287 | 101 | 1322 | 13 |
| | D52-like | | | | |
| 19 | lipocortin (LIP) | M62898 | 375 | 3697 | 10 |
| | 2 pseudogene | | | | |
| 20 | hypothetical | NM 024056 | 224 | 1310 | 6 |
| | protein | | | | |

### Example 4

### Diagnostic Accuracy of AGR2 for Detection of Metastatic Disease in Paraffin-Embedded Mediastinal Lymph Nodes (MLNs) is very High

Rationale: The microarray results derived in Example 3 were generated using NSCLC cell lines. It was important to determine whether these results were relevant to metastatic NSCLC tissue. Thus, an experiment was designed whereby expression levels of AGR2 were measured and compared to genes of high diagnostic value for detection of metastatic NSCLC disease. Studies were performed with paraffin tissues. Secondary and tertiary objectives of this study were to determine the size of section necessary for real-time RT-PCR measurements, and to determine the concordance of immunohistochemical (IHC) and real-time RT-PCR results. Pathology-negative samples were evaluated using a combination of measurements to determine markers that most accurately detected micrometastatic disease. Finally, because important information regarding cancer progression can be obtained by identifying groups of genes that are co-regulated, correlation of expression of various genes to AGR2 in cancer cell lines was assessed.

Design: 20 and 50μ FFPE sections were procured from 1) negative control MLN obtained from lung transplant patients (n=13), 2) primary tumor tissue from NSCLC patients (n=20), 3) pathology-positive MLN (n=20), and 4) pathology-negative MLN (n=20). From each tissue block, two 5μ sections were also obtained and placed on slides and used for IHC analysis; one section was stained with a cytokeratin mix, while the other was stained with an antibody to EpCam. RNA from each section was isolated and converted to cDNA using gene-specific primers to the following genes: EpCam, AGR2, lunx, CEA, PDEF, Trim29, muc1, and β₂-microglobulin (reference control gene).

For concordance analysis between IHC and real-time RT-PCR results, it was determined that a given gene was PCR “positive” if it was expressed at ≧3 standard deviations beyond the mean of normal tissue for the pathology positive samples, and ≧2 standard deviations for the pathology negative samples. The purpose was to determine whether similar or different rates of marker positivity were observed for 20μ and 501μ sections obtained from the same tissue block.

Next, pathology-negative samples were evaluated to determine markers that most accurately detected micrometastatic disease. Samples were subdivided into IHC-positive (n=7) and IHC-negative (n=57) categories. Although values of markers for detection of micrometastatic disease could be estimated from ROC curve analysis or efficiency calculations, it was reasoned that these approaches were insufficient for the present study due to small sample size and inadequate weighting of the IHC-positive samples. Instead, it was reasoned that a more accurate assessment of the markers for detection of micrometastatic disease could be obtained by combining: 1) concordance between IHC and molecular results; 2) sensitivity (defined as the percent of RIC-positive samples that were positive by molecular marker analysis; and 3) specificity (defined as the percent of IHC-negative samples that were negative by molecular marker analysis). A relative marker score was thus obtained by multiplying concordance×sensitivity×specificity×100.

Finally, the Cancer Genome Anatomy Project database (CGAP, National Cancer Institute, Bethesda, Md.) was queried for genes whose expression was most highly correlated with AGR2 in 60 cancer cell lines (NCI60).

Results: It was observed that expression levels of AGR2 were similar to EpCam (FIG. 5) in all tissues examined. To rigorously define the value of AGR2 for the detection of metastatic NSCLC, a receiver operator characteristic (ROC) curve analysis was performed using MEDCALC software (Mariakerke, Belgium). ROC curve analysis is the most commonly used method for assessing the accuracy of diagnostic tests (Henderson (1993) Ann. Clin. Biochem. 30:521-539). The area under the curve (AUC) values for EpCam and AGR2 were near 1.0 (Table 6), indicating that microarray analysis is a valid technique for identification of genes of high diagnostic value for detection of metastatic NSCLC.

TABLE 6
ROC curve analysis of pathology-positive NSCLC tissue compared
to normal nodes
Path positive samples

| | ROC (AUC) | Lower | Upper |
|---|---|---|---|
| Gene | value | limit | limit |
| EpCam | 1.00 | 0.92 | 1.00 |
| AGR2 | 0.99 | 0.90 | 0.99 |
| PDEF | 0.97 | 0.87 | 0.99 |
| CEA | 0.91 | 0.78 | 0.97 |
| Trim29 | 0.84 | 0.71 | 0.93 |
| lunx | 0.73 | 0.58 | 0.85 |
| muc1 | 0.72 | 0.58 | 0.83 |

It was also observed that the overall concordance of marker positivity was 92% and 94% respectively (Table 7), providing evidence for assay reproducibility. For the pathology-negative analysis, 2/4(50%) discordant pairs were obtained using the Trim29 gene. For the pathology-positive analysis, 4/6 (67%) of the discordant pairs were obtained using Trim29 and CEA genes. These results indicated that analysis of 20μ sections appears to be sufficient for detection of metastatic disease.

TABLE 7
Concordance analysis of marker positivity
between 20μ and 50μ sections

| | | 20μ section analysis | | |
|---|---|---|---|---|
| | | # PCR pos | #PCR neg | Concordance |
| Pathology-positive samples | | | | |
| 50μ section | #PCR pos | 47 | 1 | 92% |
| analysis | #PCR neg | 5 | 21 | |
| Pathology-negative samples | | | | |
| 50μ section | #PCR pas | 3 | 3 | |
| analysis | #PCR neg | 1 | 56 | 94% |

Using a relative marker score obtained by multiplying concordance×sensitivity×specificity×100 (Table 8), it was observed that of the seven samples that most highly overexpressed AGR2 in the pathology-negative group (n=57), five (of a total of seven) were IHC-positive (p=1.6E-5, chi-squared test). The results indicated that based on INC results, AGR2 is the most accurate marker for detection of micrometastatic disease.

TABLE 8
Relative accuracy of various markers for
detection of micrometastatic disease

| | | ΔCₜ | Concor- | Sensi- | Speci- | Relative |
|---|---|---|---|---|---|---|
| Rank | Gene | threshold | dance | tivity | ficity | score* |
| 1 | AGR2 | 14.8 | 89% | 71% | 93% | 59 |
| 2 | EpCam | 13.1 | 91% | 57% | 96% | 50 |
| 3 | PDEF | 16.7 | 79% | 67% | 84% | 44 |
| 4 | CEA | 18.7 | 82% | 50% | 89% | 37 |
| 5 | Trim | 15.3 | 79% | 43% | 86% | 29 |
| 6 | Mucl | 12.3 | 51% | 57% | 56% | 16 |
| 7 | Lunx | 17.5 | 84% | 0% | 96% | 0 |

*Relative score = concordance × sensitivity × specificity × 100 at the indicated threshold

The query of CGAP for genes whose expression was most highly correlated with AGR2 in 60 cancer cell lines identified three genes/sequences that had a positive correlation P value <1×10⁻¹⁰. The most highly correlated gene was S100P—the second most highly overexpressed gene identified in the microarray study described in Example 3 above.

During the course of the studies conducted as part of Example 4, it was observed that the RNA amounts in 14/66 (21%) of 50μ sections was inadequate, compared to 21/67 (31%) of the 20μ sections. “Adequate” was defined as samples for which the Cₜ value of the internal reference control β₂-microglobulin gene was <21. Although the failure rate of RNA extraction/PCR was higher in the small size fraction, the difference was not significantly significant. However, if tissue is not rate limiting, it may be preferable to isolate RNA from 50μ sections.

### Example 5

### Assessing Overexpression of a Panel of Highly Diagnostic Genes in MLN from Early Stage NSCLC Patients

Identification of a panel of genes with high diagnostic accuracy for NSCLC allows for more accurate staging of NSCLC patients. Further, because the survival rate of most NSCLC patients is low, a goal of the present example is to ultimately develop some of the genes in the panel into therapeutic targets. Thus, this study is devoted to identifying additional genes that: 1) have a high diagnostic accuracy for detection of metastatic NSCLC; and 2) encode potential therapeutic targets.

The data set for this example consist of archived formalin-fixed paraffin embedded (FFPE) MLN from early stage NSCLC patients who have undergone curative resection. A single MLN from all appropriate levels is examined by real-time RT-PCR multi-marker analysis, as well as by IHC using anti-cytokeratin and anti-EpCam antibodies. For right-sided lung cancers, levels 2 (paratracheal), 4 (azygos), and 7 (subcarinal) are included. Routine levels on left-sided tumors will include level 5 (aortopulmonary window) and level 7. Of these patients, half are selected on the basis of death within two years; the remaining half is selected on the basis of disease-free survival at least 2 years post-surgery. The multi-marker gene panel consists of EpCam, AGR2, PDEF, and markers (approximately 3) identified by microarray analysis and subsequently validated. The validation data set consists of FFPE MLN from patients who have undergone curative lung resection with a minimum two year follow-up.

Selection based on AUC values: For the 15 most highly overexpressed genes identified in the microarray analysis (excluding AGR2 and EpCcon), it is determined whether their respective AUC values are ≧0.95. The rationale for this is that additional genes identified in the microarray analysis are likely to have a high diagnostic accuracy for NSCLC, and further, some proteins encoded by the genes are expected to be useful therapeutic targets. Tissues used are the same as described in Example 3 above, and methods for gene expression are the same as in Examples 3 and 4 above.

The first level of screening includes pathology negative samples. Genes that exhibit a mean ΔCₜ value of ≦12.0 are immediately discarded from consideration. Genes whose AUC value is ≧0.95 and/or whose relative value score for detection of micrometastatic disease is ≧40 are included in the final gene panel.

Microarray analysis: A microarray analysis is then performed on the four lung cancer cell lines using Affymetrix U133“B” chips, using methods as described above for the Affymetrix U33“A” chips in Example 3. Although Affymetrix U133“A” chips were used in Example 3 above, the genes spotted on this chip are largely characterized. The U133“B” chips contain approximately the same number of genes as the “A” chips (˜20K), but are largely uncharacterized genes. It is expected that at least one valuable gene may be identified using “B” chips.

Determination of diagnostic accuracy: In the results described in Example 4, approximately 70% of the tissues were adenocarcinomas, while 30% were squamous cell carcinomas. Large cell carcinoma, which accounts for 10 to 20 percent of all cases of lung cancer, includes all carcinomas that are not classified as adenocarcinoma or squamous cell carcinoma, Typically, large cell carcinoma cells develop in the smaller bronchi or in scarred tissue around the outer edges of the lungs. These large cell carcinoma cells divide and replicate quickly, forming tumors that aggressively spread from the lungs to other parts of the body. For completeness, studies of potential diagnostic genes are performed on large cell carcinomas.

The design of this study largely follows that of Example 4 in that 50μ and 20μ sections are obtained from approximately 10 large cell carcinoma patients. Sections are obtained from both the primary tumor and from a metastatic MLN. Sections are stained with anti-cytokeratin and anti-EpCam antibodies. RNA is isolated from the sections and real-time PCR is performed with the genes used in Example 3, as well as the top 15 genes identified in the mieroarray studies. If the AUC value of a candidate gene is ≧0.90, it is considered for inclusion into the final marker panel.

Selection of gene panel for FFPE tissue analysis: The gene panel will consist of the internal reference control gene β₂-microglobulin, EpCam, AGR2, PDEF, and up to four additional NSCLC-associated genes selected from the above studies. The rationale for using a multi-marker gene panel is to provide high sensitivity of metastatic disease detection. The rationale for using β₂-microglobulin as an internal reference control gene is that this gene has proved satisfactory for preliminary analysis of NSCLC tissue. Other reference control genes such as GAPDH and β-actin may be used (Janssens et al. (2004) Mal. Diagn., 8:107-113). Selection of the gene panel is largely decided on the basis of high AUC and high relative value scores for detection of micrometastatic disease.

Establishment of positive controls using FFPE tissue culture cells: As noted above, for a clinical diagnostic test, adequate controls are necessary for thorough evaluation of real-time RT-PCR data. Tissue culture cells embedded in paraffin are used to satisfy the requirement that the positive control sample be homogeneous.

Briefly, 1×10⁷ tissue culture cells are pelleted and resuspended in buffer containing 10% formalin. An equal volume of liquefied HistoGel (Richard-Allan Scientific, Kalamazoo, Mich.) is added at 50° C. and vortexed. The solidified gel is then processed as a standard histology specimen (without wrapping in lens paper) and embedded in paraffin. Sections are then cut.

A sufficient number of CRL5807 cells are embedded in paraffin to be used as positive controls for the duration of the study. Analysis of mean standard deviation values provides an estimate of assay variability.

Measurement of expression of the gene panel in FFPE MLN tissues: Tissues used for this study consist of archived FFPE MLN from early stage NSCLC patients who have undergone curative resection. Queries of databases and/or clinical studies will be made using patient eligibility criteria that include: 1) patients who have undergone curative lung resection; 2) at least a 2-year follow-up; 3) primary tumor specimen available; and 4) if the cancer is right-sided, specimens need to be available from levels 2 (paratracheal), 4 (azygos) and 7 (subcarinal), if the cancer is left-sided, specimens need to be available from level 5 (aortopulmonary window) and level 7.

Of the patients available, 35 will be selected that have recurrent disease and 35 will be selected that are disease-free at least 2 years post-surgery. When possible, disease-free and recurrent patient groups are matched for tumor size/stage at diagnosis.

Databases are created for patient cohorts in an Excel file that includes patient demographic and clinical outcome information. Patient names are removed from all records and replaced by study numbers by the database manager. All files are stored as password protected files and contain the study ID number but are devoid of any patient number, medical record number, or dates of medical treatment/procedures.

Two 50μ sections and two 5μ sections are cut from each tissue block. One 50μ section is used for real-time RT-PCR analysis, while the remaining 50μ section is kept as a back-up. Both 5μ sections are used for IHC analysis; one slide is stained with an anti-cytokeratin mix, the other with the BerEp4 (EpCam) antibody. Real-time RT-PCR studies are performed in a blinded manner to clinical outcome.

RNA from each FFPE sample is isolated as described elsewhere herein. Real-time RT-PCR is performed on ABI instruments (model numbers 7300, 7500). For each PCR analysis, two controls are included: a no-template control (NTC), and a positive PCR control (described above).

SYBR Green I chemistry will be used for all reactions. The rationale for the use of this chemistry is that primer sets for all genes have been thoroughly tested using SYBR Green. All real-time data will be converted to Excel files.

Control conditions (no template control, endogenous RNA control, and positive control) are used as described in Example 2 above. Algorithms for prediction of clinical outcomes are also used as described in Example 2 above.

Validation of gene panel assay: Validation data consists of FFPE MLN from 70 consecutive patients who have undergone curative resection for NSCLC. Eligibility criteria for the patient samples and rationale for the sample size are the same as described above for the measurement of expression of the gene panel in FFPE MLN tissues. Patient identification, tissue procurement, RNA processing, real-time RT-PCR analysis, data management, and data analysis methods are also as described above. If the algorithm is able to discriminate between the two outcome groups at a p value <0.05, it is concluded that the assay is of clinical value.

RT-PCR analysis of primary tumors: Due to concerns over false positive rates associated with inadequate sampling of the entire lymph node population, primary tumor samples are also analyzed to determine whether the prognostic value of MLN analysis can be increased by RT-PCR analysis of primary tumors. Primary tumors from the patients used above for this experiment are analyzed by real-time RT-PCR using a gene panel consisting of AGR2, S100P, and 8 other genes determined to be prognostic for survival in NSCLC patients (Endoh et al. (2004) J. Clin. Oncol. 22:811-819).

### Example 6

### Identification of Informative Molecular Markers for the Detection of Micrometastatic Breast Cancer Using a Microarray Strategy

Rationale: Microarray analysis has proven to be a powerful tool for studying the mRNA expression profiles of normal and neoplastic tissues. However, the ability of this technology to identify informative molecular markers for the detection of micrometastatic disease has been limited. One major limitation of microarray analysis is that it is only semiquantitative. Thus, it is often difficult to determine which of several hundred candidate genes are likely to be most informative for detection of micrometastatic disease.

In the present example, it was hypothesized that the identification of informative markers for the detection of micrometastatic disease could be simplified by dilution of metastatic tissue (or RNA) into an excess of normal tissue (or RNA). For these analyses, RNA from a metastatic lymph node was extracted and serially diluted into a pool of normal lymph node RNA at ratios of 1:50, 1:2,500, and 1:125,000.

Materials and Methods: For microarray analysis, a metastatic ALN in which mam was overexpressed at a level 5.3×10⁷-fold higher than the mean expression in normal lymph nodes was used. In addition, four normal lymph nodes were used. Quality and quantification of RNA was assessed by an Agilent 2100 Bioanalyzer System (Agilent Technologies, Inc., Palo Alto, Calif.). RNA from the metastatic lymph node was diluted into a pool of normal lymph node RNA at ratios of 1:50, 1:2,500, and 1:125,000. For all of these conditions, expression values were obtained for a total of 22,283 gene transcripts spotted on an Affymetrix U133A army. Total cellular RNA was isolated as follows: ≦0.15 g of lymph node tissue was homogenized in 1 mL of RNA STAT-60 (TEL-TEST, Friendswood, Tex.) using a model 395 type 5 polytron (Dremel, Racine, Wis.). Total RNA isolation was done as per manufacturer's instructions up to the aqueous phase separation. Aqueous phase containing RNA was removed from organic phase and mixed with an equal volume of 70% ethanol. The sample was then loaded into an Rneasy Mini column (Qiagen, Valencia, Calif.) and purified according to the manufacturer's protocol. The RNA pellet was dissolved in 50 μL of RNase-free water.

Expression levels of 22,283 gene transcripts were determined on oligonucleotide microarrays using: (a) pooled RNA from four normal lymph nodes, (b) RNA from an ALN with a large breast cancer metastasis, and (c) RNA from an ALN with a large breast cancer metastasis diluted into pooled normal lymph node RNA at dilutions of 1:50, 1:2,500, and 1:125,000. Eight micrograms of total RNA per sample was used for microarray analysis. First- and second-strand cDNA synthesis, double-stranded cDNA cleanup, biotin-labeled cRNA synthesis, cleanup, and fragmentation were done according to protocols in the Affymetrix GeneChip Expression Analysis technical manual (Affymetrix®, Santa Clara, Calif.). Microarray analysis was done by the DNA Microarray and Bioinformatics Core Facility at the Medical University of South Carolina using U133A GeneChips (Affymetrix). Fluorescent images of hybridized microarrays were obtained by using a HP GeneArray scanner (Affymetrix). For normalization, the microarray office suite was used such that all fluorescence values were multiplied by a factor that resulted in a mean fluorescent score for all genes equal to 150.

Real-time reverse transcription-PCR validation of the dilutional microarray analysis was conducted using frozen tissue samples. Twenty H&E (+) ALN, 40 control cervical lymph nodes, and 72H&E (−)/PCR (+) ALN were used in this study. Frozen tissue specimens were obtained as part of the Minimally Invasive Molecular Staging of Breast Cancer Trial (MIMS), a prospective cohort study designed to define the clinical significance of molecular detection of micrometastatic breast cancer in ALN (Gillanders et al. (2004) Ann. Surg., 239:828837; discussion 837-840, incorporated by reference herein in its entirety). mRNA sequences of genes identified in this study were retrieved from the National Center for Biotechnology Information database. Intron-spanning primers were designed and tested in breast cancer cell lines MDA-MB-231 or SK-BR-3 (see Gillanders et al. (2004) Ann. Surg., 239:828-837). cDNA was made from 5 μg of total RNA using 200 units of Moloney murine leukemia virus reverse transcriptase (Promega, Madison, Wis.) and 0.5 μg Oligo (dT) 12-16 in a reaction volume of 20 μL (10 minutes at 70° C., 50 minutes at 42° C., and 15 minutes at 70° C.). Real-time RT-PCR analysis was done on a PE Biosystems Gene Amp 5700 Sequence Detection System (Foster City, Calif.). The standard reaction volume was 10 μL and contained 1× QuantiTect SYBR Green PCR Master Mix (Qiagen), 0.1 unit AmpErase UNG enzyme (PE Biosystems); 0.7 μL cDNA template; and 0.25 μmol/L of both forward and reverse primer. The initial step of PCR was 2 minutes at 50° C. for AmpErase UNG activation, followed by a 15-minute hold at 95° C. Cycles (n=40) consisted of a 15-second denaturation step at 95° C. followed by a 1-minute annealing/extension step at 60° C. The final step was a 60° C. incubation for 1 minute. All reactions were done in triplicate.

Real-time reverse transcription-PCR validation of the dilutional microarray analysis was also conducted on paraffin-embedded tissue samples. A 20- to 50-μ section was cut from nine H&E (+) ALN tissue blocks for mRNA extraction following the method of Specht et al. as described elsewhere herein. An adjacent 5-μ section was cut for standard H&E staining and examined by a pathologist to confirm the presence or absence of metastatic breast cancer. Briefly, paraffin-embedded tissue sections were de-paraffinized twice with 1 mL of xylene at 37° C. or room temperature for 10 minutes. The pellet was subsequently washed with 1 mL of 100%, 90%, and 70% of ethanol and air-dried at room temperature for 2 hours. The pellet was resuspended in 200 μL of RNA lysis buffer [2% lauryl sulfate, 10 mmol/L Tris-HCl (pH 8.0), and 0.1 mmol/L EDTA] and 100 μg of proteinase K and incubated at 60° C. for 16 hours. RNA was extracted using 1 mL of phenol/chloroform (5:1) solution (Sigma, St. Louis, Mo.). The aqueous layer containing RNA was transferred to a new 1.5-mL tube. Phenol/chloroform extraction was done a total of three times. RNA was precipitated with an equal volume of isopropanol, 0.1 volume of 3 mol/L sodium acetate, and 100 μg of glycogen at −20° C. for 16 hours. After centrifugation at 12,000 rpm for 15 minutes (4° C.), the RNA pellet washed with 70% of ethanol and air-dried at room temperature for 2 hours. Finally, the pellet was dissolved in 12 μL of DEPC water. cDNA synthesis was done as described above with an exception that 500 ng of a panel of truncated gene-specific primers were used instead of oligo(dT) 12-16. Truncated gene-specific primers for reverse transcription were designed as described in Mikhitarian et al. (2005) Clin. Cancer. Res., 11:3697-3704.

Results: As described above, a microarray analysis was conducted whereby RNA isolated from a highly metastatic (breast cancer) ALN was diluted into normal lymph node RNA. Candidate breast cancer-associated genes from this analysis were then selected based on the following criteria: (a) absence of expression in the pooled normal lymph nodes, (b) a fluorescence signal that was above 500 relative units for the undiluted breast cancer sample, and (c) a fluorescence signal that was present in the 1:50 dilution. The percent of genes that met each respective criterion were 52%, 8.1%, and 52%. Median relative fluorescent value for all genes was 74. Seventy-one genes were identified by criteria (a) and (b), whereas 34 genes were identified by criteria (a), (b), and (c). The 34 genes were sorted by relative intensity of metastatic signal, and the top 15 selected (see Mikhitarian et al. (2005) Clin. Cancer. Res., 11:3697-3704). Of note, of the 34 genes identified by criteria (a), (b), and (c), only mam and TFF1 had fluorescence signals above 1,000 fluorescent units in the 1:50 dilution. These results suggested that both the mam and TFFJ genes may be informative molecular markers for the detection of micrometastatic breast cancer. The gene with the highest relative intensity was mam, a result that is consistent with results from the MIMS Trial, where mam was noted to be the molecular marker that was most highly expressed in ALN containing metastatic breast cancer, as well as being the most informative marker for the detection of micrometastatic breast cancer (Gillanders et al. (2004) Ann. Surg., 239:828-837).

A closer examination of the results of the microarray analyses in this study confirms limitations of a standard (undiluted) microarray approach to gene identification. Using an undiluted sample, the fluorescence signal for mam was 6,348 (see Gillanders et al. (2004) Ann. Surg., 239:828-837), compared to 1,335 in the 1:50 dilution, 38 at the 1:2,500 dilution, and at background levels in the 1:125,000 dilution. However, based on real-time RT-PCR measurements, it was determined that mam was overexpressed in this particular ALN at a level 5.3×10⁷-fold higher than the mean expression in normal lymph nodes. It can be concluded, therefore, that without dilution, mam is in the saturated range, whereas at the 1:50 and 1:2,500 dilutions, mam is at the upper and lower end of the linear detection range, respectively. These findings demonstrate that for highly expressed genes, the hybridization signal at the undiluted level is likely to become saturated and is unlikely to be proportional to gene copy number, as opposed to the hybridization signal at diluted levels.

### Example 7

### Identification of a Core Set of Genes Required for Growth of Metastatic Cells in Multiple Cancer Types

Although epithelial-specific markers such as cytokeratin 19 (CK1 9) and epithelial cell adhesion molecule 1 (EpCAM1) are widely used for detection of metastatic disease in lymph nodes (Camp et al. (2005) Breast J., 11:394-397; Ge et al. (2005) J. Cancer Res. Clin. Oncol., 131:662-668; Heeren et al. (2005) Eur. J. Surg. Oncol., 31:270-276), bone marrow (Braun et al, (2000) N. Engl. J. Med., 342:525-533; Braun et al. (2005) N. Engl. J. Med., 353:793-802), and peripheral blood (Rao et al. (2005) Int. J. Oncot, 27:49-57; Smirnov et al. (2005) Cancer Res., 65:4993-4997; Cristofanilli et al. (2004) N. Engl. J. Med., 351:781-791) in a variety of cancer types, the relationship between these genes and other metastasis-associated genes is poorly understood but is of considerable interest due to the need to treat metastatic disease.

Multiple approaches have been used by various investigators to identify genes associated with metastatic disease. These include the analysis of primary tumor tissues associated with or without lymph node metastases (Talantov et al. (2005) Clin. Cancer Res., 11:7234-7242; Tsumagari et al. (2005) Breast Cancer, 12:166-177; Roepman et al. (2005) Nat. Genet., 37:182-186; Kan et al. (2004) Ann. Surg. Oncol., 11:1070-1078; Hoang et al. (2004) J. Thorac. Cardiovasc. Surg., 127:1332-1342; Kwon et al. (2004) Dis. Colon Rectum, 47:141-152; Nagata et al, (2003) Int. J. Cancer, 106:683-689; Huang et al. (2003) Lancet, 361:1590-1596; Kikuchi et al. (2003) Oncogene, 22:2192-2205), the comparison of poorly metastatic cell lines to those that are highly metastatic (Tarbe et al. (2002) Anticancer Res., 22:2015-2027; Zhang et al. (2002) Cancer, 95:1663-1672; Kawamata et al. (2003) Cancer Sci., 94:699-706; Kluger et al. (2005) Cancer Res., 65:5578-5587), and the comparison of primary tumors to matched metastatic tissues in mouse (Chu et al. (2006) Cancer Lett., 233:79-88; Zou et al. (2004) J. Clin. Endocrinol, Metab., 89:6146-6154) or human (Vigneswaran et al. (2005)1 Oral Pathol. Med., 34:77-86; Hao et al. (2004) Cancer, 100:1110-1122; Mori et al. (2002) Surgery, 131:S39-47) systems. In contrast to these approaches, it was decided to focus on differences in gene expression between normal lymph node and metastatic lymph nodes arising from different epithelial cancer types. A combination of microarray analysis and a novel data mining technique was used to identify a core set of genes required for growth of metastatic cells in multiple cancer types. Two clusters of genes are described, of which the Ets transcriptional factor Esx, and the secreted protein AGR2, may be critical regulators of important steps of the metastatic process.

Materials and Methods

Cell lines. CRL5807 (bronchioalveolar carcinoma), CRL5876 (adenocarcinoma), A549 (adenocarcinoma), and HTB177 (large cell carcinoma) were obtained from American Type Culture Collection (Rockville, Md.) and grown according to manufacturer's instructions.

Metastatic Lymph Nodes from Breast or Pancreatic Cancer Patients.

Breast cancer. Two metastatic axillary lymph nodes were obtained from two patients enrolled in the prospective breast cancer study previously described (Gillanders et al. (2004) Ann. Surg., 239:828-840). Both axillary lymph nodes were positive by hematoxylin and eosin (H&E) staining. Nodes were selected on the basis of previous real-time PCR analysis (Mikhitarian et al. (2005) Clin. Cancer Res., 11:C-704) indicating little or no expression of the mammaglobin (mam) gene but overexpression of at least one other cancer-associated gene (PDEF, CEA, CK19, PIP, muc1) at three standard deviations beyond the mean of normal controls. Microarray results from one axillary node in which mammaglobin was expressed at a high level were available from a previous study (Mikhitarian et al. (2005) Clin. Cancer. Res., 11:C-704).

Pancreatic cancer metastatic lymph nodes. Three metastatic lymph nodes were obtained from two pancreatic cancer patients after approval from the Medical University of South Carolina Institutional Review Board. Nodes were selected on the basis of positive staining by H&E.

Primary pancreatic cancer tumor specimens. Primary cancer tumor specimens were purchased from the Cooperative Human Tissue Network (CHTN), Southern Division (Birmingham, Ala.) and approved by the Medical University of South Carolina Institutional Review Board and CHTN Committee Board.

Immunohistochemistry. Immunohistochemical studies for epithelial antigen and keratins were performed on two adjacent 5μ sections of formalin-fixed, paraffin-embedded tissue using the labeled streptavidin-biotin method in a Dako Autostainer (Dako Corporation, Carpinteria, Calif.). One slide was stained with the Ber-EP4 monoclonal mouse anti-human antibody directed against the EpCAM1 antigen. The other slide was stained with a mix of mouse anti-human monoclonal antibodies (AE1/AE3) directed against various cytokeratin proteins. To enhance immunostaining, sections were digested with Proteinase K (Dako Corporation) for 5 minutes. The immunostaining was performed using the LSAB2 peroxidase kit (Dako Corporation). The antigen-antibody complex was visualized using 3,3′-diaminobenzidine tetrachloride as chromogen, and then counterstained with hematoxylin, To evaluate the specificity of the antibodies, known positive and negative tissues were used as controls.

Affymetrix U133A GeneChip microarray analysis. Expression levels of 22,283 gene transcripts were determined on oligonucleotide microarrays using RNA prepared from the four NSCLC cell lines described above, metastatic lymph nodes (three from breast cancer patients and three from pancreatic cancer patients), and three primary tumors from pancreatic cancer patients. Eight μg of total RNA per sample was used. First and 2^{nd} strand cDNA synthesis, double stranded cDNA cleanup, biotin-labeled cRNA synthesis, cleanup and fragmentation were performed according to protocols in the Affymetrix GeneChip Expression Analysis technical manual (Affymetrix, Santa Clara, Calif.). Microarray analysis was performed by the DNA Microarray and Bioinformatics Core Facility at the Medical University of South Carolina using U133 A GeneChips (Affymetrix, Santa Clara, Calif.). Fluorescent images of hybridized microarrays were obtained by using a HP GeneArray scanner (Affymetrix, Santa Clara, Calif.). For normalization, the microarray office suite was used such that all fluorescence values were multiplied by a factor that resulted in a mean fluorescent score for all genes equal to 150. Data for normal lymph nodes were obtained from a previous study (Mikhitarian et al. (2005) Clin. Cancer Res., 11:C-704).

Selection of most highly expressed genes in lung, breast, and pancreatic cancer. All microarray results were imported into single Microsoft excel file. The first algorithm in the selection of highly expressed genes involved elimination of genes from breast, lung, and pancreatic cancer that were expressed in normal lymph nodes (n=11,326, 50.8% of total (22,283)). The following selections were then independently performed for each cancer type:

NSCLC cell line data. Of the remaining 10,957 genes, those that were present in at least 2 NSCLC cell lines were first selected (n=1731; =7.7% of total). Subsequently, genes whose mean fluorescence in all cell lines was >500 were selected (n=91; =0.41% of total). The final group of 91 genes was sorted according to mean cell line fluorescence/mean fluorescence of normal lymph nodes.

Breast metastatic lymph node data. Of the remaining 10,957 genes, those that were present in at least 2 nodes were selected (n=1396; =6.3% of total). Genes with a mean fluorescence value >500 were next selected (n=86; =0.39% of total). The final group of 86 genes was sorted according to mean breast metastatic node fluorescence/mean fluorescence of normal lymph nodes.

Pancreatic metastatic lymph node data. Of the remaining 10,957 genes, those for which the mean fluorescence of the primary tumor was >200 units were first selected (n=455; =2.0% of total). Following, those genes containing >200 fluorescence units in at least two nodes were selected (n=143; 0.64% of total). The final group of 143 genes was sorted according to mean pancreatic metastatic node fluorescence/mean fluorescence of normal lymph nodes.

Real-time reverse transcription-PCR of formalin-fixed paraffin-embedded samples. A 20- or 50-μ section was cut from tissue blocks for mRNA extraction following the method of Specht et al. (Specht et al. (2001) Am. J. Pathol., 158:419-429). Three adjacent 5-μ sections were also cut from each block; one was used for standard H&E staining and examined by a pathologist to confirm the presence or absence of metastatic NSCLC, the other two were used for IHC. For isolation of RNA, paraffin-embedded tissue sections were de-paraffinized twice with 1 mL of xylene at 37° C. or room temperature for 10 minutes. The pellet was subsequently washed with 1 mL of 100%, 90%, and 70% of ethanol and air-dried at room temperature for 2 hours. The pellet was resuspended in 200 μL of RNA lysis buffer [2% lauryl sulfate, 10 mmol/L Tris-HC1 (pH 8.0), and 0.1 mmol/L EDTA] and 100 μg of proteinase K and incubated at 60° C. for 16 hours. RNA was extracted using 1 mL of phenol/chloroform (5;1) solution (Sigma, St. Louis, Mo.). The aqueous layer containing RNA was transferred to a new 1.5-mL tube. Phenol/chloroform extraction was done a total of three times. RNA was precipitated with an equal volume of isopropanol, 0.1 volume of 3 mol/L sodium acetate, and 100 μg of glycogen at −20° C. for 16 hours. After centrifugation at 12,000 rpm for 15 minutes (4° C.), the RNA pellet washed with 70% of ethanol and air-dried at room temperature for 2 hours. Finally, the pellet was dissolved in 12 μL of DEPC water. cDNA synthesis was performed using a panel of truncated gene-specific primers (Table 9). Real-time RT-PCR was performed on a PE Biosystems Gene Amp® 7300 or 7500 Sequence Detection System (Foster City, Calif.). With the exception of the SYBR Green I master mix (purchased from Qiagen, Valencia, Calif.), all reaction components were purchased from PE Biosystems. Standard reaction volume was 10 μl and contained 1×SYBR RT-PCR buffer, 3 mM MgCl₂, 0.2 mM each of dATP, dCTP, dGTP, 0.4 mM dUTP, 0.1 U UngErase enzyme, 0.25 U AmpliTaq Gold, 0.35 μ1 cDNA template, and 50 nM of oligonucleotide primer. Initial steps of RT-PCR were 2 min at 50° C. for UNG erase activation, followed by a 10-min hold at 95° C. Cycles (n=40) consisted of a 15 sec melt at 95° C., followed by a 1 min annealing/extension at 60° C. The final step was a 60° C. incubation for 1 min. All reactions were performed in triplicate. Threshold for cycle of threshold (Cₜ) analysis of all samples was set at 0.5 relative fluorescence units. Primer sequences for lunx, CK19, CEA5, mucl, PDEF, and β₂-microglobulin genes were previously described (Mitas et al. (2003) J. Mol. Diag., 5:237-242). Primer sequences for the remaining genes described in this example are listed in Table 9.

TABLE 9
Primer sequences used for real-time RT-PCR

| | | | |
|---|---|---|---|
| | | Ampli- | |
| | | con | |
| | | size, | Accession |
| Gene | Sequence of Primer Pairs^{a} | nt | # |

| | | | |
|---|---|---|---|
| CEA6 | AGATTGCATGTCCCCTGGAA | 104 | NM_002483 |
| | (SEQ ID NO:3) | | |
| | CATTGAATGGCGTGGATTCA | | |
| | (SEQ ID NO:4) | | |
| Claudin7 | TGGCCATCAGATTGTCACAGAC | 88 | NM_001307 |
| | (SEQ ID NO:5) | | |
| | CCAGCCAATAAAGATGGCAGG | | |
| | (SEQ ID NO:6) | | |
| EpCam1 | CGCAGCTCAGGAAGAATGTG | 88 | NM 002354 |
| | (SEQ ID NO:7) | | |
| | TGAAGTACACTGGCATTGACGA | | |
| | (SEQ ID NO:8) | | |
| Epcam2 | ACCCGAGGAGAAGAGGAGTTTG | 100 | NM 002353 |
| | (SEQ ID NO:9) | | |
| | GCTTCTTTCCCAGTGACAAGCA | | |
| | (SEQ ID NO:10) | | |
| ESRRα | AAGACAGCAGCCCCAGTGAAT | 138 | NM_004451 |
| | (SEQ ID NO:11) | | |
| | TCGGTCAAAGAGGTCACAGAGG | | |
| | (SEQ ID NO:12) | | |
| Esx | TCTTCCCCAGCGATGGTTTT | 124 | NM 004433 |
| | (SEQ ID NO:13) | | |
| | TTGCTCTTCTTGCCCTCGA | | |
| | (SEQ ID NO:14) | | |
| Mal2 | GTCTGCCTGGAGATTCTGTTCG | 103 | NM 052886 |
| | (SEQ ID NO:15) | | |
| | TCACGGACACAAACATGACCC | | |
| | (SEQ ID NO:16) | | |
| S100P | GACGTCTTTCCCGATATTCGG | 127 | NM 005980 |
| | (SEQ ID NO:17) | | |
| | CCACGGCATCCTTGTCTTTTC | | |
| | (SEQ ID NO:18) | | |
| Spint2 | GTGCCTCAAGAAATGTGCCACT | 81 | NM_021102 |
| | (SEQ ID NO:19) | | |
| | ACAGAGGAATCCGCTGCATTC | | |
| | (SEQ ID NO:20) | | |
| AGR2 | GCAGAGCAGITIGTCCTCCTCA | 76 | NM 006408 |
| | (SEQ ID NO:1) | | |
| | GGACATACTGGCCATCAGGAGA | | |
| | (SEQ ID NO:2) | | |

^{a}Upper sequence of each primer pair = forward sequence, while lower represents reverse sequence.
Underlined nucleotides = primer sequence used for reverse transcription

Results

To test the hypothesis that a core set of genes is consistently associated with metastasis in multiple cancers, three separate microarray analyses were first performed whereby expression values of a pool of normal lymph nodes (n=4) was compared to 1) four lung cancer cell lines, and 2), three metastatic lymph nodes each from breast and pancreatic cancer patients. Separate lists of the 35 most highly overexpressed genes for each cancer type were compiled (Tables 10-12; n=87 genes total) as described in the Materials and Methods. EpCAM1, AGR2, CK19, and CK8 were common to all three lists (Table 13; p=1.1E-18). Five additional genes were common to the breast and pancreatic cancer lists, while six additional genes were common to the breast and lung cancer lists (Table 13). No additional genes were common to the pancreatic and lung cancer lists (Table 13).

TABLE 10
Genes highly expressed in metastatic breast cancer disease

| Gene information | | | Fluorescence values | | | Ratio |
|---|---|---|---|---|---|---|
| Rank | Name | Acc. # | Br1 | Br2 | Br3 | Br/NORM |
| 1 | CK19 | NM_002276 | 2991 | 5974 | 4948 | 2899 |
| 2 | XAG | NM_006408 | 1571 | 4285 | 1756 | 1335 |
| 3 | S100P | NM_005980 | 1852 | 1891 | 72 | 374 |
| 4 | Mucin1 | AI610869 | 1289 | 907 | 3283 | 285 |
| 5 | FXYD | NM_005971 | 1325 | 853 | 2121 | 256 |
| 6 | Claudin3 | NM_001306 | 199 | 1303 | 168 | 232 |
| 7 | CEA6 | NM_002483 | 1810 | 79 | 839 | 111 |
| 8 | RAI3 | NM_003979 | 314 | 1603 | 585 | 106 |
| 9 | KRT7 | NM_005556 | 679 | 622 | 542 | 101 |
| 10 | SCNN1A | NM_001038 | 249 | 1476 | 511 | 86 |
| 11 | ALB | NM_000477 | 2941 | 30 | 16 | 83 |
| 12 | Unknown | AA156240 | 108 | 1436 | 526 | 79 |
| 13 | TFF1 | NM_003225 | 5443 | 195 | 5516 | 79 |
| 14 | PDEF | NM_012391 | 424 | 927 | 826 | 55 |
| 15 | EpCAM2 | NM_002353 | 1292 | 771 | 2412 | 53 |
| 16 | EpCAM1 | NM_002354 | 346 | 1529 | 1002 | 44 |
| 17 | EFNA1 | NM_004428 | 209 | 708 | 935 | 41 |
| 18 | CPB1 | NM_001871 | 588 | 196 | 7485 | 41 |
| 19 | SERPINA3 | NM_001085 | 1514 | 1840 | 6570 | 40 |
| 20 | PIP | NM_002652 | 1376 | 2707 | 1791 | 40 |
| 21 | TFF3 | NM_003226 | 4243 | 225 | 4605 | 34 |
| 22 | CYP2B6 | NM_000767 | 1968 | 19 | 255 | 32 |
| 23 | DLG5 | NM_004747 | 187 | 359 | 1455 | 32 |
| 24 | GPR56 | NM_201524 | 456 | 387 | 664 | 31 |
| 25 | CK8 | NM_002273 | 917 | 2007 | 1223 | 31 |
| 26 | Unknown | AK000345 | 40 | 1595 | 59 | 29 |
| 27 | FOS | NM_005252 | 1783 | 116 | 475 | 29 |
| 28 | CRABP2 | NM_001878 | 547 | 2094 | 1068 | 27 |
| 29 | JDP1 | NM_021800 | 174 | 534 | 836 | 27 |
| 30 | GBA | NM_000157 | 149 | 201 | 1239 | 25 |
| 31 | IGFBP2 | NM_000597 | 859 | 1679 | 297 | 24 |
| 32 | Hypothetical | BC002449 | 1052 | 2576 | 537 | 24 |
| 33 | DDR1 | NM_001954 | 491 | 1084 | 993 | 21 |
| 34 | PRSS8 | NM_002773 | 249 | 1382 | 520 | 20 |
| 35 | EEF1A2 | NM_001958 | 217 | 179 | 1354 | 18 |

TABLE 11
To E35 most highly overexpressed genes in lung cancer cell lines

| | | | | HTB | CRL | CRL | Lung | Lung/ | Lung | Mean |
|---|---|---|---|---|---|---|---|---|---|---|
| Rank | Gene | Acc# | A549³ | 177^{a} | 5807^{a} | 5876^{a} | Norm^{b} | Br′ | Pane^{d} | ratio′ |
| 1 | AGR2 | NM 006408 | 2124 | 2053 | 3082 | 38 | 960 | 0.7 | 3.3 | 2.0 |
| 2 | S100P | NM 005980 | 242 | 2522 | 2673 | 4819 | 754 | 2.0 | 13.9 | 8.0 |
| 3 | CK19 | NM 002276 | 27 | 935 | 1995 | 810 | 589 | 0.2 | 0.5 | 0.3 |
| 4 | NQO1 | NM 000903 | 1375 | 1858 | 982 | 315 | 404 | 14.5 | 11.7 | 13.1 |
| 5 | MET | NM 000245 | 1420 | 790 | 2429 | 378 | 348 | 32.1 | 17.5 | 24.8 |
| 6 | MAGE-A6 | NM 005363 | 73 | 37 | 3004 | 4475 | 311 | 131.4 | 58.8 | 95.1 |
| 7 | XAGE-1 | NM 020411 | 471 | 2 | 2322 | 3 | 250 | 344.0 | 25.9 | 184.9 |
| 8 | KRTHB1 | NM 002281 | 2822 | 31 | 221 | 3 | 208 | 37.8 | 12.2 | 25.0 |
| 9 | MAGE-A3 | NM005362 | 116 | 29 | 4055 | 5107 | 178 | 123.8 | 37.8 | 80.8 |
| 10 | MAP7 | NM 003980 | 455 | 466 | 381 | 930 | 116 | 3.7 | 29.1 | 16.4 |
| 11 | AKR1B10 | N114_020299 | 11662 | 10603 | 17 | 75 | 101 | 128.6 | 39.2 | 83.9 |
| 12 | CK7 related | N114_005556 | 537 | 21 | 1319 | 463 | 96 | 1.0 | 8.2 | 4.6 |
| 13 | EpCAM2 | NM 002353 | 2 | 3 | 8146 | 2342 | 94 | 1.8 | 2.2 | 2.0 |
| 14 | EpCAMI | NM 002354 | 278 | 15 | 4430 | 3244 | 91 | 2.1 | 11.4 | 6.8 |
| 15 | P-cadherin | NM_001793 | 2 | 3 | 1319 | 1274 | 87 | 16.4 | 3.9 | 10.2 |
| 16 | EFNA1 | NM_004428 | 72 | 209 | 354 | 4301 | 81 | 2.0 | 23.4 | 12.7 |
| 17 | Unknown | AA156240 | 78 | 121 | 421 | 2088 | 78 | 1.0 | 3.8 | 2.4 |
| 18 | GPCR5A | N114_003979 | 203 | 112 | 514 | 1523 | 74 | 0.7 | 1.6 | 1.2 |
| 19 | DHRS2 | AK000345 | 68 | 11 | 5377 | 52 | 71 | 2.4 | 18.2 | 10.3 |
| 20 | Uroplakin Ib | N114_006952 | 61 | 9 | 20 | 1951 | 64 | 22.1 | 4.7 | 13.4 |
| 21 | Junctate | AF306765 | 2960 | 4417 | 3650 | 4459 | 51 | 20.9 | 30.2 | 25.5 |
| 22 | BM039 | NM_018455 | 341 | 583 | 563 | 524 | 48 | 16.3 | 5.8 | 11.0 |
| 23 | Interleukin 8 | NM_000584 | 218 | 4427 | 246 | 235 | 48 | 35.0 | 4.4 | 19.7 |
| 24 | TOM1L1 | NM_005486 | 579 | 481 | 806 | 503 | 39 | 2.8 | 4.3 | 3.6 |
| 25 | FER1L3 | N11, 4_013451 | 1420 | 2552 | 2016 | 3298 | 34 | 5.4 | 7.5 | 6.4 |
| 26 | LC27 | NI14_018407 | 706 | 710 | 3144 | 1910 | 32 | 6.2 | 53.9 | 30.1 |
| 27 | PSG1 | M34421 | 42 | 31 | 46 | 7179 | 28 | 27.7 | 8.3 | 18.0 |
| 28 | Midline 1 | NM 000381 | 1241 | 518 | 556 | 723 | 27 | 18.4 | 9.0 | 13.7 |
| 29 | FGG | N111_000509 | 1974 | 90 | 36 | 16 | 26 | 28.3 | 6.7 | 17.5 |
| 30 | ANX3 | NM_005139 | 486 | 380 | 1931 | 798 | 26 | 10.4 | 7.3 | 8.9 |
| 31 | Myosin X | NM_012334 | 1244 | 251 | 1445 | 336 | 25 | 10.6 | 5.5 | 8.0 |
| 32 | COL5A2 | N11, 4000393 | 91 | 2748 | 19 | 15 | 23 | 3.0 | 6.2 | 4.6 |
| 33 | MAGE-A12 | N114_005367 | 13 | 70 | 2352 | 1722 | 23 | 35.5 | 4.1 | 19.8 |
| 34 | Laminin B1 | M20206 | 666 | 1161 | 404 | 317 | 22 | 7.1 | 3.3 | 5.2 |
| 35 | CK8 | NM_002273 | 473 | 105 | 810 | 2567 | 22 | 0.7 | 1.7 | 1.2 |

^{a}Normalized fluorescent values obtained from Affymetrix U133A array data for the indicated cell line.
^{b}Ratio of mean NSCLC cell line data to mean of normal lymph node.
^{c}Ratio of mean NSCLC cell line data to mean of metastatic breast lymph node.
^{d}Ratio of mean NSCLC cell line data to mean of metastatic pancreatic lymph node.
^{e}Mean of ^{c} and ^{d}.

TABLE 12
Top 35 most highly overexpressed genes in metastatic pancreatic cancer lymph nodes

| | | | | | | Pane/ | Pane/ | Pane/ | |
|---|---|---|---|---|---|---|---|---|---|
| Rank | Gene | Acc # | Met1 | Met2 | Met3 | Norm | BR | Lung | Mean |
| 1 | PNLIPRP2 | BC005989 | 342 | 30 | 10121 | 1943 | 2440.0 | 896.7 | 1668.4 |
| 2 | CK19 | NM 002276 | 572 | 1082 | 4205 | 1221 | 0.4 | 2.1 | 1.2 |
| 3 | AGR2 | NM006408 | 1093 | 539 | 36 | 292 | 0.2 | 0.3 | 0.3 |
| 4 | FXYD | NM_005971 | 1242 | 300 | 653 | 131 | 0.5 | 1.7 | 1.1 |
| 5 | SGP28 | NM_006061 | 227 | 16 | 298 | 129 | 20.2 | 17.8 | 19.0 |
| 6 | CEA6 | N114_002483 | 41 | 678 | 989 | 69 | 0.6 | 1.2 | 0.9 |
| 7 | Unknown | AB020676 | 102 | 296 | 16566 | 64 | 34.6 | 29.0 | 31.8 |
| 8 | Mucin 1 | A1610869 | 909 | 247 | 63 | 63 | 0.2 | 11.6 | 5.9 |
| 9 | Unknown | AB028949 | 16 | 317 | 5447 | 55 | 5.9 | 61.4 | 33.7 |
| 10 | MMP19 | NM 002429 | 320 | 205 | 208 | 43 | 13.5 | 29.2 | 21.3 |
| 11 | ANX14 | NM 007193 | 248 | 296 | 822 | 31 | 25.3 | 12.6 | 18.9 |
| 12 | FOSB | NA4_006732 | 400 | 530 | 43 | 29 | 1.2 | 66.5 | 33.9 |
| 13 | ACACA | NM000664 | 257 | 226 | 1608 | 29 | 2.2 | 1.8 | 2.0 |
| 14 | Rhodanese | D8729 | 393 | 306 | 189 | 25 | 1.5 | 3.4 | 2.4 |
| 15 | CD151 antigen | NM_004357 | 620 | 53 | 384 | 24 | 0.8 | 5.0 | 2.9 |
| 16 | GCNT3 | NM 004751 | 232 | 271 | 349 | 23 | 24.2 | 2.6 | 13.4 |
| 17 | Hypothetical | NM 02514 | 248 | 69 | 3083 | 20 | 26.6 | 35.1 | 30.9 |
| 18 | Endothelin 3 | NM 000114 | 201 | 213 | 101 | 19 | 17.3 | 15.9 | 16.6 |
| 19 | NR4A1 | NM 002135 | 403 | 560 | 128 | 16 | 3.6 | 18.0 | 10.8 |
| 20 | CK8 | NM002273 | 390 | 1003 | 385 | 13 | 0.4 | 0.6 | 0.5 |
| 21 | GPX2 | NA/1_002083 | 563 | 1034 | 111 | 13 | 5.9 | 1.5 | 3.7 |
| 22 | SPINK1 | NM_003122 | 287 | 284 | 121 | 12 | 27.9 | 7.4 | 17.6 |
| 23 | GARP | NM 005512 | 93 | 368 | 277 | 12 | 2.8 | 38.9 | 20.8 |
| 24 | Galectin 4 | NM006149 | 588 | 1560 | 92 | 11 | 13.2 | 28.5 | 20.8 |
| 25 | Unknown | A1479175 | 234 | 128 | 422 | 11 | 0.9 | 11.1 | 6.0 |
| 26 | Unknown | AA143765 | 707 | 240 | 9 | 10 | 1.6 | 2.5 | 2.1 |
| 27 | FOS | NA/1_005252 | 202 | 456 | 187 | 10 | 0.4 | 6.4 | 3.4 |
| 28 | SERPINA3 | NM_001085 | 812 | 442 | 1047 | 9 | 0.2 | 18.7 | 9.5 |
| 29 | Hypothetical | NM_006820 | 241 | 320 | 56 | 9 | 4.0 | 7.0 | 5.5 |
| 30 | UPAR | U08839 | 407 | 690 | 781 | 9 | 3.1 | 2.0 | 2.6 |
| 31 | MYH11 | NM_022844 | 558 | 938 | 770 | 8 | 4.0 | 125.4 | 64.7 |
| 32 | EpCAM1 | NM 002354 | 72 | 216 | 236 | 8 | 0.2 | 0.1 | 0.1 |
| 33 | CK17 | Z19574 | 345 | 501 | 11 | 8 | 3.1 | 3.5 | 3.3 |
| 34 | Unknown | BE500977 | 264 | 65 | 1309 | 8 | 1.2 | 12.7 | 6.9 |
| 35 | AEBP1 | NA/1_001129 | 525 | 138 | 1130 | 7 | 1.5 | 73.5 | 37.5 |

^{a}Normalized fluorescent values obtained from Affymetrix U133A array data.
^{b}Ratio of mean pancreatic metastatic lymph node to mean of normal lymph node.
^{c}Ratio of mean pancreatic metastatic lymph node to mean of metastatic breast lymph node.
^{d}Ratio of mean pancreatic metastatic lymph node to mean of NSCLC cell line data.
^{e}Mean of ^{c} and ^{d}.

TABLE 13
Genes present in two or more of the top 35 lists in Tables 10-12

| Breast, lung, | Breast and | Breast and | Pancreas and |
|---|---|---|---|
| and pancreas | pancreas only | lung only | lung only |
| CK8 | CEA6 | CK7-related | — |
| CK19 | FXYD | DHRS2 | — |
| EpCAM1 | Muc1 | EFNA1 | — |
| AGR2 | SERPINA | EpCAM2 | — |
| — | FOS | GPCR5A | — |
| — | | S100P | — |

AGR2 is Highly Overexpressed in Metastatic NSCLC

Overexpression of EpCAM1 and cytokeratin genes has previously been observed in metastatic NSCLC (Wallace et al. (2005) Chest, 127:430-437; Mitas et al. (2003) Clin. Chem., 49:312-315) esophageal cancer (Xi et al. (2005) Clin. Cancer Res., 11:1099-1109), and breast cancer (Mitas et al. (2001) Int. J. Cancer, 93:162-171). Although the AGR2 gene was recently shown to promote breast cancer metastasis in an in vivo mouse model system (Liu et al. (2005) Cancer Res., 65:3796-3805), overexpression of this gene in lymph nodes containing metastatic NSCLC disease has not been reported. To determine whether AGR2 was overexpressed in metastatic NSCLC, real-time RT-PCR was used to quantitate the expression of AGR2 in formalin fixed, paraffin embedded (FFPE) control negative mediastinal lymph nodes (MLN; n=24), and in pathology-positive (H&E+) MLN (n=27) derived from NSCLC patients. For comparative purposes, primary tumors from NSCLC patients (n=30), pathology-negative, NC-negative (H&E−/IHC−) MLN (n=44), and H&E−, IHC-positive (H&E/IHC+) MLN (n=4) were also analyzed. All sections were quantitated for the expression of AGR2, as well as five other genes (EpCAMI, Lunx, PDEF, CEA5, and mucl) previously determined to be useful for the detection of metastatic disease in MLN (procured by endoscopic ultrasound-guided fine needle aspiration; EUS-FNA) (Wallace et al. (2005) Chest, 127:430-437).

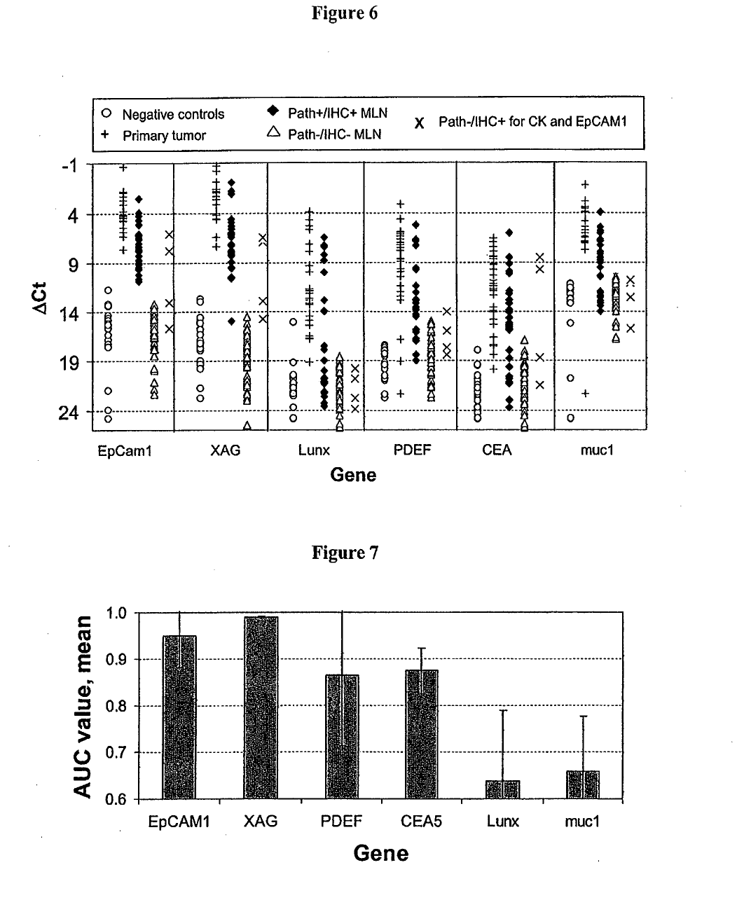

Real-time RT-PCR results indicated that expression levels of EpCAMI, AGR2, and mucl were higher in all primary tumor samples compared to normal lymph nodes (FIG. 6). Expression of an individual gene in primary tumor samples was not correlated with that measured in H&E+ lymph nodes. EpCAMI and AGR2 levels were higher in 27/27 (100%) and 26/27 (96%; respectively) of the H&E+ samples compared to the H&E−/IHC− group. These results suggest that EpCAMI and AGR2 may be tightly associated with the metastatic phenotype in NSCLC. In further support of this association, it was observed that AGR2 expression was high in the four samples that were H&E−/IHC+ (FIG. 6). To compare the ability of the various genes to detect macro- (H&E+) and micro- (H&E−/IHC+) metastatic NSCLC, ROC curve analysis was performed (Henderson (1993) Ann. Clin. Biochern., 30:521-539) for each gene comparing 1) control negative samples versus H&E+ samples, and 2), H&E−/IHC− samples versus H&E−/IHC+ samples. The mean AUC value for AGR2 (0.991+/−0.001) was significantly higher compared to CEA5, lunx, and mucl (FIG. 7), providing evidence that AGR2 is a gene associated with NSCLC macro- and micro-metastatic disease. Interestingly, the AUC value of AGR2 for detection of micro metastatic disease (95% CI=0.912-0.994) was significantly higher compared to PDEF (95% CI=0.619-0.866), a gene recently shown to work in conjunction with receptor tyrosine kinases (RTK) Her2 and colony-stimulating factor receptor (CSF-1R)/CSF-1 to enhance epithelial cell migration and invasion (Gunawardane et al. (2005) Cancer Res., 65:11572-11580).

The results described above support the model that a core set of genes is tightly associated with metastatic growth in multiple epithelial cancers. Next, it was sought (1) to determine whether EpCAM1, AGR2, CK19, and CK8 formed an exclusive core of metastatic-associated genes or constituted a subset of a larger group of core genes, and (2) to determine whether this core group of genes was coordinately regulated by specific transcription factors. To address both issues, the on-line Comparative Genome Anatomy Project (CGAP) NCI60 gene expression database (URL=http://cgap.nci.nih.gov/) was queried using all 87 genes identified from the three lists. In this query, the output consists of a list of 10 genes whose expression levels are most highly correlated with the single gene query sequence. Using the output from each gene, a correlation map was constructed such that the appearance of a gene on the map required: 1) high correlation (p<8.0E-6) with at least two other genes; and 2) direct or indirect contact to EpCAM1 or AGR2. Genes identified from the first set of queries were used as query in a reiterative round of interrogation (data mining).

Two Gene Clusters Associated with Metastatic Disease are Linked by the Ms Transcription Factor Esx/Elf3

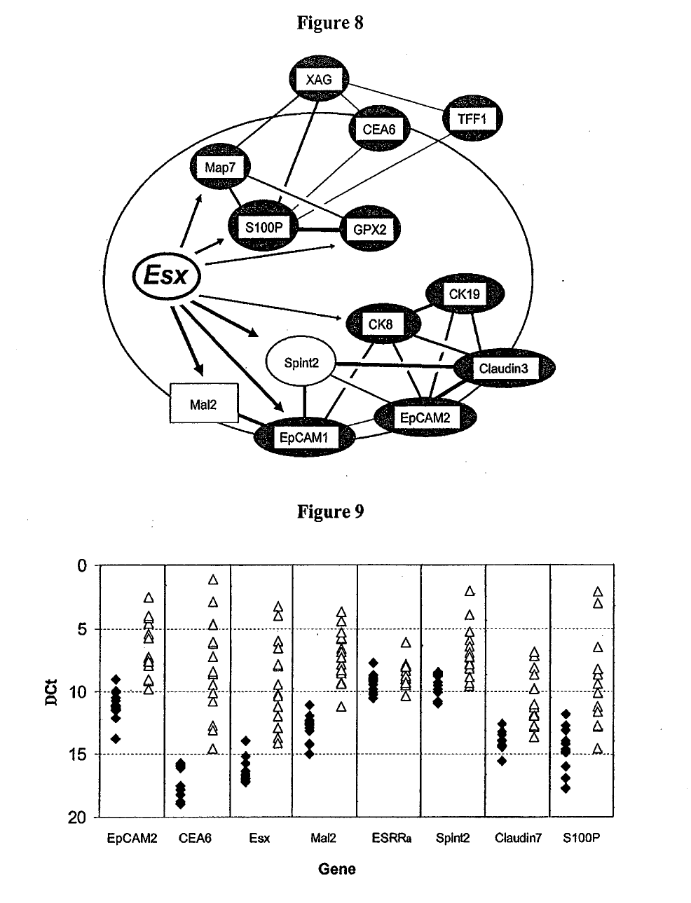

The resultant correlation map contained 13 genes in two distinct clusters (AGR2 cluster and EpCAM1 cluster) that were joined together by a single gene (Esx; FIG. 8).

The AGR2 cluster consisted of six genes, two of which encoded secreted proteins (AGR2, TFF1), three cytosolic proteins (Map7, S100P, GPX2) and one membrane-bound protein (CEA). Interestingly, the CEA gene contained on the map was family member 6 and not 5, the gene used in the real-time RT-PCR study described above (FIG. 6) and also used in the CALGB 9761 lung cancer trial (D'Cunha et al. (2002) J. Thorac. Cardiovasc. Surg., 123:484-491). Among the genes in the AGR2 cluster, the two whose levels of expression were most highly correlated with one another were gastrointestinal glutathione peroxidase (GPX2) and S100P; the correlation coefficient (R²) between these two genes was good (R²=0.85; p=1.4E-17).

The EpCAM1 cluster consisted of seven genes, four of which encoded membrane-bound proteins (EpCAM1, EpCAM2, Mal2, and Claudin3) and three cytosolic proteins (CK8, CK19, and Spint2). Five genes in the cluster were identified from the microarray analysis (EpCAM1, EpCAM2, CK8, CKI 9, Claudin3), while one gene (Mal2) was not present on the Affymetrix U133A array. Among the genes in the EpCAMI cluster, the two whose expression levels were most highly correlated with one another were Spint2 and Claudin3 (R²=0.85; p=1.5E-17).

Perhaps the most significant finding regarding the correlation map was the observation that the AGR2 and EpCAM1 clusters were directly connected via the Ets transcription factor Esx. Expression of Esx was highly correlated with three members of the AGR2 cluster (Map7, S100P, GPX2) and four members of the EpCAMI cluster (Mal2, Spint2, EpCAMI, and CK8). Esx also exhibited the highest number of connections to other genes (n=7; genes listed above); the gene exhibiting the second highest number of connections was S100P (n=6).

To establish the clinical relevance of these findings to NSCLC, the hypothesis that genes within these two clusters, as well as Esx, were overexpressed in metastatic FFPE MLN obtained from NSCLC patients (n=15) was tested. MLN obtained from 13 lung transplant patients were used as negative control. RNA was isolated and converted to cDNA using sequence-specific primers to six genes contained on the correlation map (EpCAM2, CEA6, Esx, Ma12, Spint2, S100P), as well as Claudin7 and ESRRα, a gene known to regulate the expression of TFFI (Lu et al. (2001) Cancer Res., 61:6755-6761; Barry & Giguere (2005) Cancer Res., 65:6120-6129; Barry et al. (2005) MoL. Endocrinol, doi: 10.1210/me.2005-0313). Analysis of real-time RT-PCR expression data (FIG. 9) by ROC curve analysis indicated that AUC values for four of six genes on the correlation map (EpCAM2, CEA6, Esx, Ma12) were greater than 0.98 (see Example 9 below). This result provides strong evidence that the six genes listed on the correlation map are highly associated with metastatic NSCLC disease.

Conclusions

In the present study, separate lists of the 35 most highly over-expressed genes for each cancer type were compiled (n=87 genes total). Each list contained EpCAM1, AGR2, CK19, and CK8 (p=1.1E-18), suggesting a tight association between the expression of these genes and the spread or maintenance of metastatic disease in multiple cancers. This is the first report that AGR2 is involved in NSCLC. AGR2 was initially identified as a cement gland-specific gene with a putative role in ectodermal patterning in Xenopus, being expressed in the anterior region of dorsal ectoderm from late gastrula stages onwards. Activation of AGR2 transcription is observed in response to organizer-secreted molecules including the noggin, chordin, follistatin and cerberus gene products (Aberger et al. (1998) Mech. Dev., 72:115-130), The AGR2 gene is co-expressed with the estrogen receptor in breast cancer cell lines (Fletcher et al. (2003) Br. J. Cancer, 88:579-585; Thompson & Weigel (1998) Biochem. Biophys. Res. Commun., 251:111-116) or primary tumors (Abba et al. (2005) B.M.C. Genomics, 6:37) and is also induced by androgen stimulation in prostate cancer (Mang et al. (2005) Genes Chromosomes Cancer, 43:249-259). More importantly, the AGR2 gene causes an increase in the number of lung metastases derived from breast primary tumors in a rat model system (Liu et al. (2005) Cancer Res., 65:3796-3805). The DAG1 gene, whose membrane-bound protein product was recently shown to interact directly with AGR2 (Fletcher et al. (2003) Br. J. Cancer, 88:579-585), was also highly expressed in two of the three cancers (75th and 80^{th} most highly expressed gene in metastatic breast cancer and NSCLC, respectively). That AGR2 is highly expressed in both NSCLC and pancreatic cancer suggests that AGR2 is activated by non-steroidal mechanisms.

Genes on the Correlation Map are also Overexpressed in the Rat Pancreatic Metastatic Cell Line BSp73-ASML

Using the 35 most highly expressed genes from each epithelial cancer type, a novel correlation map of genes whose expression levels were highly correlated with one another in the NCI60 CGAP database was constructed. The resultant map contained two gene clusters that were linked by Esx (or Elf3, Esel) (Neve et al. (2006) Gene, 367:118-25 (Epub, Nov. 22, 2005); Hou et al. (2004) Gene, 340:123-131; Neve et al. (2002) Oncogene, 21:3934-3938; Neve et al. (1998) FASEB J., 12:1541-1550). Due to the relatively stringent criteria used for construction of the map, it was hypothesized that all genes in the map would be highly expressed in multiple epithelial cancers, a conclusion that is supported by the high diagnostic accuracy of many of the genes on the map for detection of metastatic NSCLC (FIG. 9) and esophageal cancer.

Previous studies by Tarbe et al. identified genes that were overexpressed in a highly metastatic rat pancreatic cell line (BSp73-ASML) compared to its non-metastatic parent (BSp73-AS) (Tarbe et al. (2002) Anticancer Res., 22:2015-2027). After s.c. injection, Bsp73-ASML colonizes the lymph nodes and metastasizes to the lungs. Of the twelve genes that were expressed at >350-fold levels higher in BSp73-ASML, five (EpCAM1, EpCAM2, CK8, GPX2, and Claudin3) were included in the 14-gene correlation map shown in FIG. 8 (p<1.0E-50; chi-squared test). This finding provides strong evidence that the genes identified in the present study are involved in the spread of metastatic disease and adds further validation to the novel approach that has been taken. Also, of the nine genes in the correlation map that were not expressed >350-fold higher in BSp73-ASML, six (AGR2, Esx, Map7, S100P, CEA6, Mal2) were not among the 7,000 genes included in the Affymetrix U34 rodent array used by Tarbe et al.

In further support of the genes listed on the correlation map, studies by Zoller and colleagues have also shown that the metastatic BSp73-ASML cell line (but not the non-metastatic parent) expresses a complex located in glycolipid-enriched membrane microdomains that contains EpCAM1 (Schmidt et al. (2004) Exp. Cell Res., 297:329-347) and phosphorylated tight junction protein Claudin7 (Ladwein et al. (2005) Exp. Cell Res., 309:345-357). The EpCAM-Claudin7 complex is also observed in colorectal cancers. Claudins 1-4 are known to bind membrane type metalloproteinases (MMP) and pro-MMPs, thus providing a focus of MMP activation (Miyamori et al. (2001) J. Biol. Chem., 276:28204-28211). It is thought that the EpCAM1-Claudin7 complex might contribute to cell motility (Ladwein et al. (2005) Exp. Cell Res., 309:345-357), an event that is frequently associated with tetraspanin complexes (Berditchevski (2001) J. Cell Sci., 114:4143-4151). In the present report, it was observed that Claudin 7 was overexpressed in metastatic MLN obtained from NSCLC patients (FIG. 9).

P-Cadherin, Rather than E-cadherin, may Play a Major Role in Maintenance of the Metastatic Phenotype

Interestingly, claudin3, and not claudin7, was present on the correlation map. The gene that was most highly correlated with claudin3 expression in the NCI60 database was EpCAM2 (R²=0.85; p=1.5E-17), a gene resulting from retro-transposition of EpCAM1 (Fornaro et al. (1995) Mt. J. Cancer, 62:610-618). It is tempting to speculate that EpCAM2 might form a complex with Claudin3 that functions to activate MMPs in a manner akin to EpCAM1-Claudin 7 complex contribution to cell motility (Ladwein et al. (2005) Exp. Cell Res., 309:345-357). Interestingly, in the NCI60 database, EpCAM2 expression inversely correlated with expression of twist1 (R²=0.61; p=2.8E-7), a gene that contributes to metastasis by promoting EMT through suppression of E-cadherin-mediated cell-cell adhesion (Yang et al. (2004) Cell, 117:927-939). Of the Epithelial, Neural, Heart, and Placental-cadherin genes, EpCAM2 and EpCAM1 expression measured in these studies was correlated most highly with P-cadherin (R²=0.56 and 0.92, respectively), a gene that was present in the NSCLC top 35 gene list (Table 11), but virtually absent in all normal tissue in the NCI60 database. In the CGAP NCI60 database, P-cadherin expression was weakly correlated with claudin7 (R²=0.54) and C4.4A (R²32 0.57), a gene whose protein product binds directly to AGR2 (Fletcher et al, (2003) Br. J. Cancer, 88:579-585). With respect to the four cadherin genes analyzed in the present study, EpCAM2 expression was correlated least with E-cadherin (R²=0.01). This finding provides evidence that lymph node metastatic disease is molecularly distinguishable from primary tumor tissue, which is characterized by high E-eadherin expression (Bogenrieder & Herlyn (2003) Oncogene, 22:6524-6536). Due to the inverse correlation between EpCAM2 and twist1 expression levels, it is likely that the markers listed on the correlation map, including Esx, are activated during, or subsequent to, MEC. In the context of lymph node metastases, it is possible that co-expression of P-cadherin and Esx (and/or additional genes) promotes both the spread and growth of metastatic lesions.

In contrast to cluster 2 (FIG. 8) which contained 4 membrane-bound proteins (Mal2, EpCAM1, EpCAM2, and Claudin3) and no secreted proteins, cluster 1 only contained 1 membrane bound protein (CEA6) and two secreted proteins (TFF1 and AGR2). Based on the difference in ratios of secreted to membrane bound proteins, clusters 1 and 2 are statistically different from one another (p=0.005; chi-squared test). Although little is known regarding the functions of many of the genes in cluster 1, several have been previously implicated in the spread of metastatic disease. For example, CEA6 is involved in adhesion, invasion, and metastasis of human colon cancer (Blumenthal et al. (2005)Cancer Res., 65:8809-8817), while the Ca⁺⁺-binding protein S100P (Wang et al. (2006) Cancer Res., 66:1199-1207) has been shown to promote metastases in breast and pancreatic cancer (Arumugam et al. (2005) Clin. Cancer Res., 11:5356-5364). Further, high expression of S100P is a strong predictor of distant metastasis and survival in early-stage NSCLC (Diederichs et al. (2004) Cancer Res., 64:5564-5569).

Potential Interaction of AGR2, Esx, and Her2 Pathway Genes

In the correlation map, gene clusters 1 and 2 were joined by Esx, an Ets transcription factor whose expression is restricted to the most terminally differentiated epithelial-derived cells in multiple organs (Neve et al. (1998) FASEB. J., 12:1541-1550; Bochert et al. (1998) Biochem. Biophys. Res. Commun., 246:176-181; Tymms et al. (1997) Oncogene, 15:2449-2462). Upregulation of Esx in breast cancer cells lines is known to modulate expression of many genes important in tumor progression such as Her2, TGF-B RII, MIP-3 alpha, nitric oxide synthase, and collagenase (Eckel et al. (2003) DNA Cell Biol., 22:79-94). Consequently, Esx overexpression can confer a metastatic phenotype to MCF12A normal human epithelial cell lines as evidenced by increasing colony formation in soft agar mediated by a novel cytoplasmic mechanism (Prescott et al. (2004) Mol. Cell Biol., 24:5548-5564). Conversely, decreasing Esx in the breast cancer cell line T47D results in decreased colony formation. The transactivating domain of Esx has been shown to interact with DRIP130/CRSP130/Sur-2, a Ras-linked metazoan-specific subunit of human mediator complexes (Asada et al. (2002) Proc. Natl. Acad. Sci. USA, 99:12747-12752). Disruption of the interaction between Esx and DRIP130 by a short cell-permeable peptide reduces the expression of Her2 and impairs the growth and viability of Her2-overexpressing breast cancer cells. In the microarray data generated in this report, it was observed that Her2 expression was positively correlated with both AGR2 and CK19 (R²=0.72 and 0.80, respectively), thus providing evidence that expression of AGR2, Esx and Her2 may be interrelated.

As described above, 14 genes have been identified that may be critical for maintenance of the metastatic phenotype in breast, lung, and pancreatic cancer. Two of these genes (TFF1 and AGR2) are secreted proteins and are also highly expressed in estrogen receptor-positive tumors (Thompson & Weigel (1998) Biochem. Biophys. Res. Cotnmun., 251:111-116; Sun et at (2005) Exp. Cell Res., 302:96-107). Five genes are membrane-bound proteins; three are involved in cell adhesion (EpCAM1, EpCAM2, CEA6); and one (Claudin3) may interact with one or more of the EpCAM proteins. Six genes encode cytosolic proteins, including two cytoskeletal genes (CK8 and CK19), one Ca⁺⁺ binding protein (S100P) and glutathione peroxidase. One of the 14 genes is a transcriptional factor that may regulate expression of one or more of the remaining 13 genes. The genes identified on the correlation map should be useful for diagnosis and/or prognosis of many cancer types and may also be viable therapeutic targets.

### SUMMARY

Microarray analysis was combined with a novel data mining technique to identify a core set of genes that are tightly associated with metastatic disease in multiple cancer types. Three separate Affymetrix U133A microarray analyses were first performed whereby expression values of a pool of normal lymph nodes was compared to four lung cancer cell lines, as well as three metastatic lymph nodes each from breast and pancreatic cancer patients. Separate lists of the 35 most highly overexpressed genes for each cancer type were compiled (n=87 genes total). Each list contained EpCAM1, AGR2, CK19, and CK8 (p=1.1E-18). To determine if these genes were linked by a common regulatory network, the CGAP NCI60 gene expression database was queried with the 87 genes, and a correlation map was constructed such that the appearance of a gene on the map required high correlation (p<8.0E-6) with at least two other genes, and direct or indirect “connections” to EpCAM or AGR2. The map contained 13 genes in two clusters that were connected to, and potentially regulated by, the Ets transcription factor Esx/Elf3. Of eight genes from the map that were tested by real-time RT PCR, six (EpCAM1, CEA6, EpCAM2, AGR2, Esx, Malt) were able to discriminate metastatic NSCLC lymph nodes from normal lymph nodes at >98% accuracy. The correlation map described in this report provides a better understanding of the transcriptional regulation of metastasis-associated genes as they fit into a regulatory network that may be targeted by sequential and/or combinatorial therapies.

### Example 9

### Expression of Members of Identified Gene Clusters and Esx in Metastatic Mediastinal Lymph-Nodes Obtained from NSCLC Patients

Rationale: To establish the clinical relevance of the above findings to NSCLC, the hypothesis that members of the two gene clusters identified above, as well as Esx, are overexpressed in metastatic MLN obtained from NSCLC patients was tested.

Design: 20 or 50μ formalin fixed, paraffin-embedded (FFPE) sections of MLN were obtained from: 1) lung transplant patients (n=13; negative controls); and 2), NSCLC patients with pathology-positive nodes (n=15). RNA from each section was isolated and converted to cDNA using gene-specific primers to the following genes: EpCAM1, pCAM2, AGR2, CEA6, Esx, Mal2, ESRRα Spint2, and Claudin 7 (the protein product of this gene binds directly to EpCAM1), and β₂-microglobulin (reference control gene). Results were then analyzed by ROC curve analysis using MedCalc software.

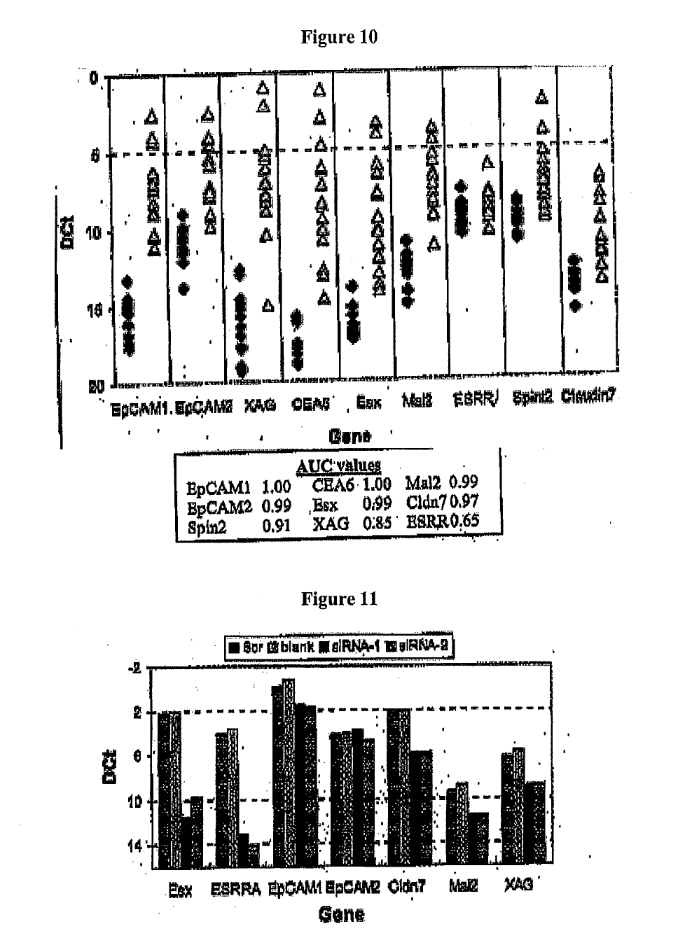

Results: The AUC value of 5/7 (71%) genes tested from the two clusters, including Esx, was >0.95 (FIG. 10), providing strong evidence that these genes are diagnostic of metastatic NSCLC disease.

### Example 10

### Treatment of the NSCLC Cell Line CRL5876 With Esx siRNA

Rationale: To determine whether Esx regulated the expression of metastasis-associated genes.

Design: CRL5876, which is derived from NSCLC lymph node metastasis, was transfected (20 nM) separately with two siRNAs to Esx, a control siRNA scrambled sequence, as well as a buffer control. Cells were harvested 48 hours following transfection to determine: 1) extent of Esx knockdown; and 2), whether any of the genes in the clusters described above were also knocked down.

Results: The scrambled siRNA had no effect on knockdown of any gene (FIG. 11). In contrast, expression of Esx was knocked down ˜200-600-fold with either of the Esx siRNAs (FIG. 11; note that ΔCₜ of 1 corresponds to a 2-fold change in gene expression levels). Both Esx siRNAs also knocked down EpCAM1 and Mal2 expression approximately 4-fold. The most profound effect was a 1000-fold knockdown of ESRRα by Esx siRNA-2. Interestingly, Claudin 7, whose protein product has previously been shown to bind directly to EpCAM1, was knocked down 14-16 fold by Esx siRNA. AGR2 expression was knocked down approximately 7-fold.

Conclusions: Esx regulates expression of genes associated with metastatic disease.

### Example 11

### Anterior Gradient 2 and TFF1 are Co-Expressed in Metastatic Breast Cancer Lymph Nodes Derived from Estrogen Receptor-Negative (ER−) Primary Tumors

Metastatic lymph nodes from breast cancer patients. Three metastatic axillary lymph nodes were obtained from three patients enrolled in the prospective breast cancer study previously described (Gillanders, et al., Ann Surg, 239(6): 828-840. (2004). Two nodes were selected based on real-time PCR analysis (Gillanders, et al., Ann Surg, 239(6): 828-840. (2004) indicating little or no expression of the mammaglobin gene but overexpression of at least one other cancer-associated gene (PDEF, CEA, CK19, PIP, muc1) at three standard deviations beyond the mean of normal controls, while one node was selected based on high mammaglobin expression.

Affymetrix® U133A GeneChip microarray analysis. Expression levels of 22,283 gene transcripts were determined on oligonucleotide microarrays using RNA prepared from metastatic lymph nodes. Eight μg of total RNA per sample was used. First and 2^{nd} strand cDNA synthesis, double stranded cDNA cleanup, biotin-labeled cRNA synthesis, cleanup and fragmentation were performed according to protocols in the Affymetrix® GeneChip Expression Analysis technical manual (Affymetrix, Santa Clara, Calif.). Microarray analysis was performed by the DNA Microarray and Bioinformatics Core Facility at the Medical University of South Carolina using U133 A GeneChips (Affymetrix, Santa Clara, Calif.). Fluorescent images of hybridized microarrays were obtained by using a HP GeneArray scanner (Affymetrix, Santa Clara, Calif.). For normalization, the microarray office suite was used such that all fluorescence values were multiplied by a factor that resulted in a mean fluorescent score for all genes equal to 150.

RNA Isolation and cDNA Synthesis. RNA was isolated from approximately 0.1 g tissue, and cDNA was synthesized as described previously by Mitas, et al., Int J Cancer, 93(2):162-171 (2001). Briefly RNA was isolated from ALN using RNA STAT-60 (Tel-Test, Friendswood, Tex.). Following the addition of 1.0 ml of RNA-STAT-60, ALN were homogenized and 0.2 ml of chloroform (Sigma, St. Louis, Mo.) was added to the mixture. Samples were centrifuged at 12,000 RPM for 15 minutes at 4° C. The aqueous phase was removed and RNA was precipitated using a 1:1 addition of isopropanol (Sigma, St. Louis, Mo.) to sample and 0.5 μl of 50 mg/ml glycogen. RNA precipitation was preformed at −20° C. for 12 hours. Following precipitation, RNA was centrifuged at 12,000 RPM for 15 minutes at 4° C. The RNA pellet was dried using 1.0 ml of 70% ethanol (Sigma, St. Louis, Mo.). Synthesis of cDNA was performed using 5 μg of total RNA and 500 ng of Oligo dT incubated for 10 minutes at 70° C. Reverse transcription was preformed using 200 units of Moloney murine leukemia virus reverse transcriptase (Promega, Madison, Wis.) in a total reaction volume of 20 μl incubated at 42° C. for 50 minutes then 70° C. for 10 minutes.

Real-time reverse transcription-PCR of metastatic lymph nodes. Real-time RT-PCR was performed on a PE Biosystems Gene Amp® 7300 or 7500 Sequence Detection System (Foster City, Calif.). With the exception of the SYBR Green I master mix (purchased from Qiagen, Valencia, Calif.), all reaction components were purchased from PE Biosystems. Standard reaction volume was 10 □l and contained 1×SYBR RT-PCR buffer, 3 mM MgCl₂, 0.2 mM each of dATP, dCTP, dGTP, 0.4 mM dUTP, 0.1 U UngErase enzyme, 0.25 U AmpliTaq Gold, 0.35 □l cDNA template, and 50 nM of oligonucleotide primer. Initial steps of RT-PCR were 2 min at 50° C. for UNG erase activation, followed by a 10-min hold at 95° C. Cycles (n=40) consisted of a 15 sec melt at 95° C., followed by a 1 min annealing/extension at 60° C. The final step was a 60° C. incubation for 1 min. All reactions were performed in triplicate. Threshold for cycle of threshold (Cₜ) analysis of all samples was set at 0.5 relative fluorescence units. Primer sequences for, CK19, CEA5, mucl, PDEF, and □₂-microglobulin genes were previously described (Mitas, et al., J Mol Diag,; 5: 237-242 (2003)). Primer sequences for the remaining genes described in this paper are listed in Table 14.

Data Analysis. Calculation of gene expression values. Briefly, mean C, values were normalized to the mean internal control (2-microglobulin) value, which yielded ΔCₜ values. Threshold values for each marker were set at 3 standard deviations below the mean Cₜ value. Correlation Analysis.

The correlation coefficients of ΔCₜ values for AGR2 to the various genes (n=13) were calculated and then tested for significance using Fisher's z test.

RNA Interference. MDA453 (American Type Culture Collection, Manassas, Va.) cells (5×10⁵) were plated in 6-Well Plates B-D Falcon (Franklin Lakes, N.J.) and grown in RPMI (CellGro, Herndon, Va.) supplemented with 5% FBS (Gibco) for 24 hours. The medium was removed and cells were transfected with 0.05 μM siRNA (Invitrogen, Carlsbad, Calif.) using 10 μl of Lipofectimine 2000 (Invitrogen) and 500 μl of Opti-MEM (Gibco, Carlsbad, Calif.) and grown in 1.5 ml of RPMI 5% FBS for 48 hours. Human AGR2 stealth siRNA (Invitrogen) sequences were 5′-GGACACAAAGGACUCUCGACCCAAA-3′ (SEQ ID NO:21) and 5′-UUUGGGUCGAGAGUCCUUUGUGUCC-3′ (SEQ ID NO:22). Human TFF1 stealth siRNA (Invitrogen) sequences were 5′-AAAUUCACACUCCUCUUCUGGAGGG-3′ (SEQ ID NO:23) and 5′CCCUCCAGAAGAGGAGUGUGAAUUU-3′ (SEQ ID NO:24). Negative Control, Medium GC (Invitrogen) was used for the scrambled control. Following 48 hours of growth, cells were harvested for RNA extraction.

Cell Invasion Assay. The cell invasion study was performed using an 8 μM pore size QCM™ 96-Well Cell Invasion Assay (Chemicon, Temecula, Calif.) following the manufacturer's instructions. Briefly, 24 hours following siRNA transfection, in triplicate, 2.0×10⁵ cells were transferred to a 96 well plate and grown in RPMI with 5% FBS using 25% FBS as a chemoattractant in the bottom chamber. Cells were grown for 24 hours and then measured for invasiveness using CyQuant Dye (Chemicon) fluorescence using a Fluoroskan Ascent (Thermo Scientific, Waltham, Mass.).

Results: To elucidate genes involved in breast cancer metastasis, RT-PCR records of >500 lymph nodes obtained from breast cancer patients in which expression levels of mammaglobin A, mammaglobin B, CK19, CEA, Muc1, PIP, and PDEF were measured were screened (Gillanders, et al., Ann Surg, 239(6): 828-840. (2004). From the available nodes, three were selected: one node was selected based on high levels of mammaglobin A gene expression, while the other two were selected on the basis of low mammaglobin A expression, and high expression of at least two of the remaining five genes listed above. The rationale for using mammaglobin expression as a selection criterion was based on the previous observation that this gene is upregulated in response to in vitro treatment of tissue culture cells with estrogen (Wilson, et al., Endocr Relat Cancer, 13(2): 617-628 (2006). RNA was isolated from each of the nodes and an Affymetrix U133A cDNA microanalysis was performed as described above. Gene expression values from the individual nodes were then compared to a pool of four normal nodes obtained from patients with no evidence of disease.

To identify those genes that were most highly expressed in metastatic nodes, genes that were expressed in normal lymph nodes (n=11,326, 50.8% of total (22,283)) were first eliminated, and then genes whose expression was detected in at least 2 nodes (n=1396; =6.3% of total) were selected. Of the remaining 1,396 genes, those genes whose mean fluorescence value was >500 (n=86; =0.39% of total) were selected. The final group of 86 genes was sorted according to mean breast metastatic node fluorescence/mean fluorescence of normal lymph nodes. The top 35 most highly expressed genes are listed in Table 10.

After CK19, the second most highly expressed gene in the metastatic nodes was anterior gradient 2 (AGR2), a gene known to be upregulated in estrogen receptor positive (ER+) tumors (Wilson, et al., Endocr Relat Cancer, 13(2): 617-628 (2006); Fletcher, et al., Br J Cancer, 88(4): 579-585 (2003); Liu, et al., Cancer Res, 65(9): 3796-3805 (2005); Thompson and Weigel, Biochem Biophys Res Commun, 251(1): 111-116 (1998). To determine the extent to which this gene was overexpressed in metastatic lymph nodes, its expression was measured in H&E positive axillary lymph nodes (n=70), of which 53 were derived from patients with ER+ primary tumors, and 17 that were derived from patients with ER− tumors. For comparison, the expression of 13 additional breast cancer-associated genes was also measured, including TFF1, ER, and PR. Expression levels of the genes were also measured in negative control cervical lymph nodes (n=9−49), and results of the expression levels were analyzed by ROC curve analysis, the most commonly used method for determining the diagnostic accuracy of test assays (Swets, Science, 240(4857): 1285-1293 (1988).

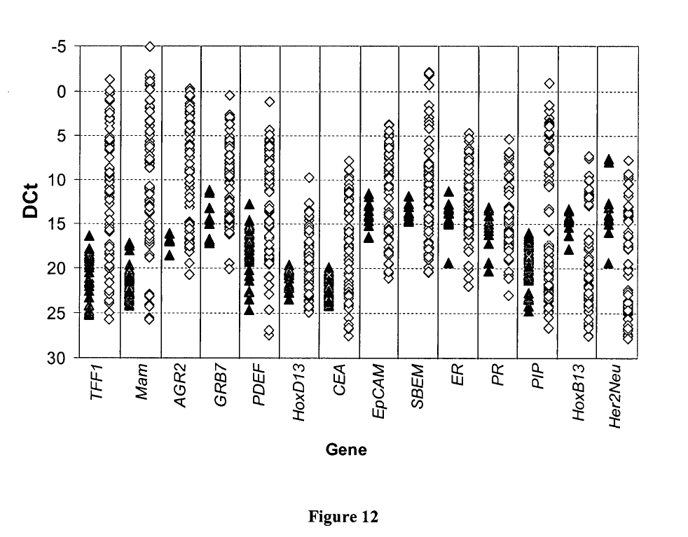

Compared to expression in normal lymph nodes, the three genes with the highest diagnostic accuracies were TFF1, mammaglobin, and AGR2 (Table 14, FIG. 12). That TFF1 and mammaglobin were highly overexpressed in metastatic lymph nodes is consistent with previous reports Bosma, et al., Clin Cancer Res, 8(6): 1871-1877 (2002); Mikhitarian, et al., Clin Cancer Res, 11(10): C-704 (2005); Smid, et al., J Clin Oncol, 24(15): 2261-2267 2006; Weigelt, et al., Br J Cancer, 90(8): 1531-1537 (2004), and (Gillanders, et al., Ann Surg, 239(6): 828-840. (2004) Berger, et al., Anticancer Res, 26(5B): 3855-3860 (2006); Corradini, et al., Ann Oncol, 12(12): 1693-1698 (2001); Fleming, et al., Ann N Y Acad Sci, 923: 78-89 (2000); Grunewald, et al., Lab Invest, 80(7): 1071-1077 (2000); Marchetti, et al., J Pathol, 195(2): 186-190 (2001); Min, et al., Cancer Res, 58(20): 4581-4584 (1998); Nissan, et al., Br J Cancer (2006); Ouellette, et al., Am J Clin Pathol, 121(5): 637-643 (2004); Watson and Fleming, Cancer Res, 56(4): 860-865 (1996); Watson, et al., Cancer Res, 59(13): 3028-3031 (1999); Zach, et al., Biotechniques, 31(6): 1358-1362 (2001), respectively). The gene that was overexpressed to the third highest level was AGR2 (AUC value=0.890, 95% CI 0.803-0.948), providing evidence that this gene is highly overexpressed in breast cancer metastatic disease.

TABLE 14

Diagnostic accuracy of various genes for detection of meta-
static disease; primers for amplification of each gene.

| | | | | | |
|---|---|---|---|---|---|
| Gene | Accession | Sequence 5′-3′ | AUC | Lower | Upper |

| | | | | | | |
|---|---|---|---|---|---|---|
| 1 | TFF1 | NM_003225 | AATGGCCACCATGGAGAACA | 0.928 | 0.849 | 0.973 |
| | | | (SEQ ID NO:25) | | | |
| | | | ACCACAATTCTGTCTTTCACGG | | | |
| | | | (SEQ ID NO:26) | | | |
| 2 | MAM | NM_002411 | CGGATGAAACTCTGAGCAATGT | 0.909 | 0.827 | 0.961 |
| | | | (SEQ ID NO:27) | | | |
| | | | CTGCAGTTCTGTGAGCCAAAG | | | |
| | | | (SEQ ID NO:28) | | | |
| 3 | AGR2 | NM_006408 | GCAGAGCAGTTTGTCCTCCTCA | 0.890 | 0.803 | 0.948 |
| | | | (SEQ ID NO:29) | | | |
| | | | GGACATACTGGCCATCAGGAGA | | | |
| | | | (SEQ ID NO:30) | | | |
| 4 | GRB7 | NM_005310 | GCTTTGTCCTCTCTTTGTGCCA | 0.823 | 0.725 | 0.897 |
| | | | (SEQ ID NO:31) | | | |
| | | | GGCCATCATCCATGCTGAAG | | | |
| | | | (SEQ ID NO:32) | | | |
| 5 | PDEF | NM_012391 | AGTGCTCAAGGACATCGAGACG | 0.797 | 0.695 | 0.877 |
| | | | (SEQ ID NO:33) | | | |
| | | | AGCCACTTCTGCACATTGCTG | | | |
| | | | (SEQ ID NO:34) | | | |
| 7 | HOXD13 | NM_000523 | TGGAACAGCCAGGTGTACTGCA | 0.756 | 0.619 | 0.863 |
| | | | (SEQ ID NO:35) | | | |
| | | | TCTTCGGTAGACGCACATGTCC | | | |
| | | | (SEQ ID NO:36) | | | |
| 8 | CEA | NM_001712 | GGGCCACTGTCGCATCATGATTGG | 0.746 | 0.640 | 0.834 |
| | | | (SEQ ID NO:37) | | | |
| | | | TGTAGCTGTTGCAAATGCTTTAAGGAAGAAG | | | |
| | | | (SEQ ID NO:38) | | | |
| 9 | EpCAM | NM_002354 | CGCAGCTCAGGAAGAATGTG | 0.688 | 0.578 | 0.784 |
| | | | (SEQ ID NO:39) | | | |
| | | | TGAAGTACACTGGCATTGACGA | | | |
| | | | (SEQ ID NO:40) | | | |
| 11 | SBEM | AF414087 | CCACTGCTCGTAAAGACATTCC | 0.651 | 0.538 | 0.752 |
| | | | (SEQ ID NO:41) | | | |
| | | | ACCAATTGCAGAAGACTCAAGC | | | |
| | | | (SEQ ID NO:42) | | | |
| 12 | ER | NM_000125 | CTTGCTCTTGGACAGGAACCA | 0.568 | 0.456 | 0.675 |
| | | | (SEQ ID NO:43) | | | |
| | | | ACCGAGATGATGTAGCCAGCAG | | | |
| | | | (SEQ ID NO:44) | | | |
| 13 | PR | NM_000926 | GCAGATGCTGTATTTTGCACCT | 0.528 | 0.416 | 0.638 |
| | | | (SEQ ID NO:45) | | | |
| | | | TCTGCCACATGGTAAGGCATA | | | |
| | | | (SEQ ID NO:46) | | | |
| 14 | PIP | NM_002652 | GCCAACAAAGCTCAGGACAAC | 0.523 | 0.411 | 0.633 |
| | | | (SEQ ID NO:47) | | | |
| | | | GCAGTGACTTCGTCATTTGGAC | | | |
| | | | (SEQ ID NO:48) | | | |
| 10 | HOXB13 | NM_006361 | TTGGAAGGCAGCATTTGCAG | — | 0.577 | 0.783 |
| | | | (SEQ ID NO:49) | | | |
| | | | TGTACGGAATGCGTTTCTTGC | 0.687 | | |
| | | | (SEQ ID NO:50) | | | |
| 6 | HER2neu | NM_004448 | GAGACCCGCTGAACAATACCAC | — | 0.655 | 0.846 |
| | | | (SEQ ID NO:51) | | | |
| | | | CTGGATCAAGACCCCTCCTTTC | 0.760 | | |
| | | | (SEQ ID NO:52) | | | |

AGR2 Loses its Correlation to ER and PR Genes in Metastatic Lymph Nodes Derived from ER− Tumors, but Maintains its High Correlation with TFF1

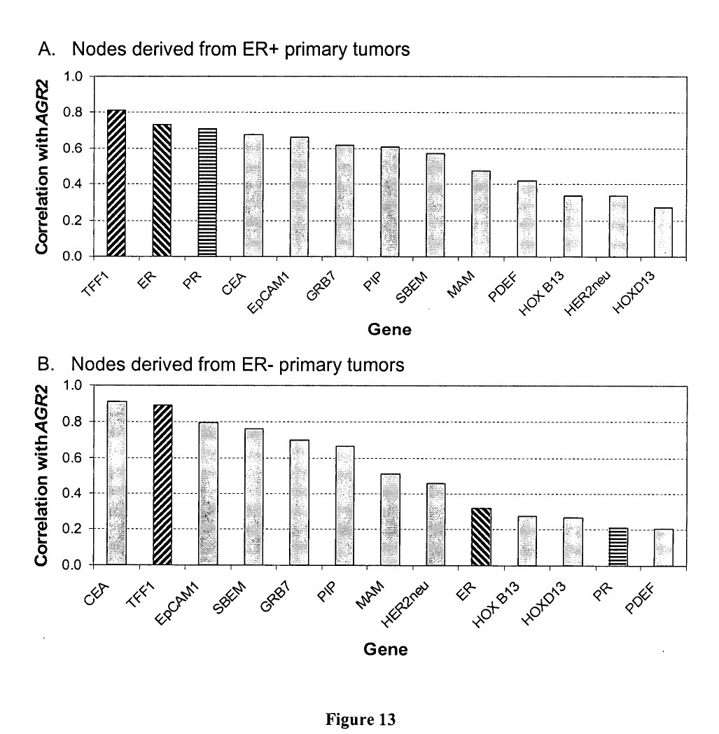

To understand the role of AGR2 in breast cancer metastatic disease, 13 genes were investigated to see whether any might be co-expressed with AGR2 in metastatic nodes derived from ER+ and ER− tumors. Based on previous data, it was hypothesized that of the 13 genes d by real-time RT-PCR, expression levels of AGR2 in nodes derived from ER+ patients would be most highly correlated with TFF1, ER, and PR. To test this hypothesis, a correlation coefficient analysis of the 53 nodes derived from ER+ patients was performed, and it was observed that TFF1, ER, and PR were the three genes (out of 13) whose expression was most highly correlated with AGR2 (P=3.5E-3; FIG. 13A). Based on the results of this analysis, it was further hypothesized that in nodes derived from ER− patients, correlation of TFF1, ER, and PR with AGR2 would significantly decrease. In partial agreement with this hypothesis, it was observed that the correlation between ER and AGR2, as well as PR and AGR2, significantly decreased in metastatic nodes derived from ER− patients (FIG. 13B). However, the correlation coefficient between AGR2 and TFF1 did not significantly change (FIG. 13B). This unexpected result provides evidence that the high correlation between TFF1 and AGR2 in metastatic nodes is independent of hormone receptor status of the primary tumor.

AGR2 Expression is Highly Correlated with TFF1 in Other Cancer Cell Types

Based on the above results, it was hypothesized that the high correlation between AGR2 and TFF1 would not be restricted to breast cancer. To test this hypothesis, the on-line Comparative Genome Anatomy Project (CGAP) NCI60 gene expression database (URL=http://cgap.nci.nih.gov/) was queried using AGR2 or TFF1. The CGAP database contains cDNA microarray results from 60 different cell lines that span 10 different cancer types. The output of a given query consists of a list of 10 genes whose expression levels are most highly correlated with the query sequence. Of the 10 genes most highly correlated with AGR2, it was observed that S100P, a gene identified by microarray analysis as one of the top 3 overexpressed genes in breast cancer metastatic disease, was ranked 1^{st}; the correlation coefficient between the two genes was 0.76 (p=1.8E-12). Among the 10 different cancer cell types, expression of AGR2 was highest in lung and colon.

Of the 10 genes most highly correlated with TFF1, it was observed that AGR2 was ranked 5^{th}; the correlation coefficient between the two genes was 0.59 (p=6.8E-7). These results provide evidence that in addition to metastatic breast cancer disease, AGR2 and TFF1 are co-expressed in other cancer types.

Knockdown of AGR2 and/or TFF1 Gene Expression in an ER-Cell Line Results in Decreased Cell Invasion

To further understand the relationship between AGR2 and TFF1 in metastatic disease, in vitro studies using the cell line MDA453 and short interfering RNAs (siRNA) were performed. Although otherwise considered as an ER-line (de Longueville, et al., Int J Oncol, 27(4): 881-892 (2005); Love, et al., Cancer Res, 56(12): 2789-2794 (1996), it was observed by real-time RT-PCR measurements that MDA453 does express ER mRNA, but at a level that is ˜10² times less compared to the ER+ cell lines MCF7. MDA453 cells were treated in triplicate with TFF1, AGR2, or a negative control scrambled siRNA to knockdown specific gene expression. Cells were harvested 48 hours following treatment and analyzed for 1) cell invasion, and 2) expression of AGR2, TFF1, and ER. Determining whether knockdown of AGR2 resulted in a decrease in expression levels of TFF1, or vice-versa, was of particular interest.

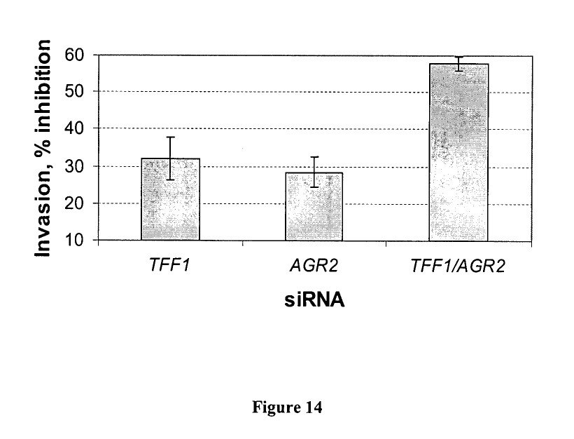

Compared to cells transfected with the negative control scrambled siRNA sequence, cells transfected with TFF1 or AGR2 exhibited a 32±6% or 28±4% decrease, respectively, in cell invasion (FIG. 14). Interestingly, co-transfection of both TFF1 and AGR2 siRNA resulted in a 57±2% decrease in cell invasion, indicating that concurrent knockdown of TFF1 and AGR2 had an additive effect resulting in a greater decrease in cell invasiveness. These results provide evidence that AGR2 and TFF1 both play a role in cell invasion in ER− cells.

Following transfection with TFF1 siRNA, quantitative real-time RT-PCR measurements revealed a 7.4-fold reduction in TFF1 gene expression, confirming that the siRNA was successfully transfected into the cells. However, transfection of TFF1 siRNA resulted in only a modest 1.4-fold reduction in AGR2, providing evidence that TFF1 does not regulate expression of AGR2. In a similar manner, cells treated with AGR2 siRNA exhibited a 4.5-fold reduction of AGR2, but only a modest 1.7-fold reduction in TFF1 expression levels. This result provides evidence that AGR2 does not significantly regulate the expression of TFF1.

In contrast to the modest effect of TFF1 siRNA on AGR2 expression levels, a significant (33-fold) and unexpected reduction of ER expression was observed. Interestingly, a similar reduction in ER expression was observed in response to treatment with TFF1 siRNA (39-fold). These results provide evidence that AGR2 and TFF1 modulate expression of genes in the ER pathway.

### Example 12

mRNA levels were measured using quantitative real-time RT-PCR for eight genes in 15 metastatic NSCLC lymph nodes (10 adeno and 5 squamous) versus normal controls (n=13) and 14 genes in esophageal (n=6), colon (n=8) and pancreas (n=12) metastatic lymph nodes compared to six normal pancreatic lymph nodes (n=6). Since the results from the metastatic esophageal, colon, and pancreatic lymph nodes were comparable, they were combined for AUC analysis. AUC values for several genes, including AGR2, was rather high (Table 15). Expression levels of AGR2 in metastatic lymph nodes obtained from breast cancer patients were also measured (n=70), and it was found that this gene was highly overexpressed (Table 14).

TABLE 15
Diagnostic accuracy of metastatic disease detection for various genes.

| Gene description | | | | Diagnostic accuracy in metastatic nodes | | | |
|---|---|---|---|---|---|---|---|
| | | Upregulated | | Pancreas, | | | |
| | Cellular | in following | | Esophagus, Colon | | NSCLC | |
| Name¹ | function | cancers² | Acc. # | AUC³ | 95% Cl | AUC | 95% Cl |
| CEA6* | Adhesion | Lu, B, P, S | NM_002483 | 1.000 | 0.867-1.000 | | |
| EpCAM1* | ″ | Lu, S, P, Li, O | NM_002354 | 0.980 | 0.852-0.992 | 1.000 | 0.867-1.000 |
| Claudin7 | ″ | T, Lu, S, P, K | NM_001307 | 0.974 | 0.832-0.992 | | |
| CDH3* | ″ | Lu, B, S, P, K, C | NM_001793 | 0.920 | 0.764-0.985 | | |
| EpCAM2* | ″ | Lu, S, P, Li, K, C | NM_002353 | 0.780 | 0.595-0.908 | 0.988 | 0.845-1.000 |
| CDH1 | ″ | Lu, P, K, Pr | NM_004360 | 0.760 | 0.573-0.894 | | |
| Spint2 | Anti- | Lu, B, P, Li, K, | NM_021102 | | | 0.908 | 0.664-0.961 |
| | peptidase | Pe, Pr, BM | | | | | |
| S100P* | Ca++ binding | Lu, B, S, P, Li, K | NM_005980 | 0.960 | 0.821-0.995 | 0.857 | 0.664-0.961 |
| CK19* | Cytoskeletal | Lu, S, P, Li, K, C | NM_002276 | 0.940 | 0.792-0.991 | | |
| MAP7* | ″ | Lu, B, S | NM_003980 | 0.853 | 0.680-0.953 | | |
| CK8* | ″ | Lu, S, P, K, Pe | NM_002272 | 0.547 | 0.359-0.725 | | |
| AGR2* | Secreted | T, B, P, Li, C | NM_006408 | 0.900 | 0.738-0.977 | 0.846 | 0.660-0.953 |
| TFF1 | ″ | Lu, P, Sk | NM_003225 | 0.713 | 0.523-0.860 | | |
| GPX2 | Peroxidase | T, P, Pr, M | NM_002083 | 0.973 | 0.841-0.994 | | |
| Esx | Transcription | Lu, B, S, P, K | NM_004433 | 0.900 | 0.738-0.977 | 0.988 | 0.845-1.000 |
| Trim29 | ″ | Lu, S, P, K | NM_012101 | | | 0.844 | 0.705-0.934 |
| Mal2 | Transcytosis | Li, P | NM_052886 | | | 0.944 | 0.856-1.000 |

¹Genes marked with asterisk were among the list of 15 most highly expressed genes in the four NSCLC cell lines.
²Upregulated in cancer tissue with respect to normal according to CGAP database. Tissue abbreviations: T, thyroid; Lu, lung; B, breast; S, stomach; P, pancreas; Li, liver; K, kidney; C, colon; Pr, prostate; Sk, skin; Pe, peritoneum.
³AUC values were obtained using MedCalc software and were derived from a pool of metastatic esophageal (n = 6), colon (n = 8), and pancreatic (n = 12) lymph nodes compared to normal lymph nodes (n = 6). A missing value indicates that the respective gene was not tested.

### Example 13

### EpCAM2 is Overexpressed in Peripheral Blood Obtained from Advanced Stage Prostate Cancer Patients

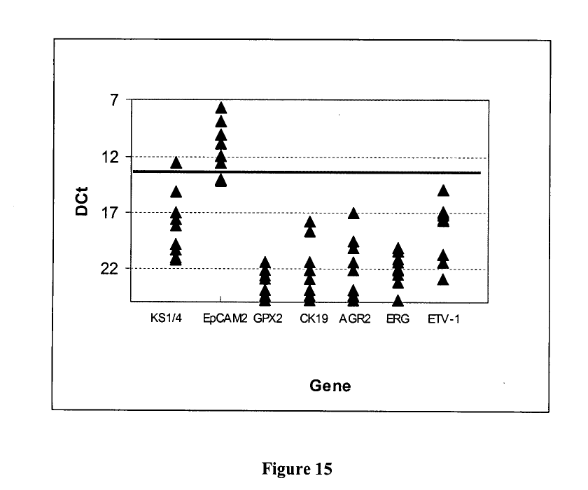

Fifteen mls. of peripheral blood was obtained from 8 advanced stage cancer patients. Blood was enriched for circulating tumor cells by the use of density gradient centrifugation as previously described (Baker M K, Mikhitarian K, Osta W, et al. Molecular detection of breast cancer cells in the peripheral blood of advanced-stage breast cancer patients using multimarker real-time reverse transcription-polymerase chain reaction and a novel porous barrier density gradient centrifugation technology. Clin Cancer Res, 9(13): 4865-4871 (2003). RNA was isolated from the cells, converted to cDNA and measured for the expression of each of the indicated genes using real-time RT-PCR. (FIG. 15)

TABLE 16

| | Other designations | Aliases | | | |
|---|---|---|---|---|---|
| mam | mammaglobin 1; mammaglobin A | MGB1 | UGB2 | SCGB2A2 | |
| AGR2 | anterior gradient 2 homolog; | AG2 | GOB-4 | HAG-2 | XAG-2 |
| | anterior gradient 2 homolog | | | | |
| | (Xenepus laevis); secreted | | | | |
| | cement gland homolog | | | | |
| TFF1 | breast cancer estrogen-inducible | BCEI | D21S21 | HP1.A | HPS2 |
| | sequence; gastrointestinal trefoil | | | | |
| | protein pS2 | | | | |
| PDEF | prostate epithelium-specific | SPDEF | RP11-375E1_A.3 | bA375E1.3 | |
| | Ets transcription factor | | | | |
| S100P | S100 calcium-binding protein P; | MIG9 | | | |
| | migration-inducing gene 9 | | | | |
| EpCam1 | MK-1 antigen; antigen identified | EpCam | CD326 | C017-1A | EGP |
| | by monoclonal antibody AUA1; human | | | | |
| | epithelial glycoprotein-2; | | | | |
| | membrane component, chromosome 4, | | | | |
| | surface marker (35 kD glycoprotein) | | | | |
| CK19 | 40-kDa keratin intermediate filament | KRT19 | K19 | K1CS | MGC15366 |
| | precursor gene; cytokeratin 19; | | | | |
| | keratin, type I cytoskeletal 19; | | | | |
| | keratin, type 1, 40-kd | | | | |
| CK8 | cytokeratin 8; keratin, type II | KRT8 | CARD2 | CYK8 | K2C8 |
| | cytoskeletal 8 | | | | |
| Esx | E74-like factor 3 (ets domain | ELF3 | EPR-1 | ERT | ESE-1 |
| | transcription factor, epithelial- | | | | |
| | specific) | | | | |
| CEA6 | carcinoembryonic antigen-related | CD66c | CEAL | NCA | CEACAM6 |
| | cell adhesion molecule 6 (non- | | | | |
| | specific cross reacting antigen) | | | | |
| GPX2 | gastrointestinal glutathione | GI-GPx | GPRP | GSHPX-GI | GSHPx-2 |
| | peroxidase 2; glutathione peroxidase- | | | | |
| | related protein 2 | | | | |
| MAL2 | MAL proteolipid protein 2; MAL2 | | | | |
| | proteolipid protein | | | | |
| Spint2 | serine peptidase inhibitor, | HAI-2 | HAI2 | Kop | PB |
| | Kunitz type, 2 | | | | |
| EpCam2 | tumor-associated calcium signal | TROP2 | EGP-1 | GA733 | GA733-1 |
| | transducer 2 [Homo sapiens]; | | | | |
| | epithelial glycoprotein-1; membrane | | | | |
| | component, chromosome 1, surface | | | | |
| | marker 1 (40 kD glycoprotein, | | | | |
| | identified by monoclonal antibody GA733) | | | | |
| Claudin3 | CPE-receptor 2; Clostridium perfringens | C7orf1 | CPE-R2 | CPETR2 | HRVP1 |
| | enterotoxin receptor 2; claudin-3; | | | | |
| | rat ventral prostate. 1-like protein | | | | |
| NQO1 | NAD(P)H dehydrogenase, quinone 1 | DHQU | DIA4 | DTD | NMOR1 |
| MET | met proto-oncogene (hepatocyte growth | HGFR | RCCP2 | | |
| | factor receptor) [Homo sapiens] | | | | |
| MAGE-A3 | melanoma antigen family A, 3; MAGE-3 | HIP8 | HYPD | MAGE3 | MAGEA6 |
| | antigen; antigen MZ2-D; melanoma- | | | | |
| | associated antigen 3 | | | | |
| XAGE-1 | X antigen family, member 1 ?? | XAGE-1b | GAGED1 | | |
| KRT81 | keratin 81; mhard keratin, type II, | HB1 | Hb-1 | KRTHB1 | MLN137 |
| | 1; keratin, hair, basic, 1 | | | | |
| Mucin1 | mucin 1, transmembrane | Mud | | | |
| FXYD 3 | FXYD domain containing ion transport | MAT-8 | MAT8 | MGC111076 | PLML |
| | regulator 3; FXYD domain-containing | | | | |
| | ion transport regulator 3; | | | | |
| | phospholemman-like protein | | | | |
| GPCR5A | protein-coupled receptor, family C, | GPCR5A | RAI3 | RAIG1 | |
| | group 5, member A; retinoic acid | | | | |
| | induced 3; retinoic acid responsive gene | | | | |
| SCNN1A | sodium channel, nonvoltage-gated 1 | ENaCa | ENaCalpha | FU21883 | SCNEA |
| | alpha; alpha ENaC-2; amiloride-sensitive | | | | |
| | epithelial sodium channel alpha subunit | | | | |
| SGP28 | cysteine-rich secretory protein 3; | CRISP3 | Aeg2 | CRISP-3 | CRS3 |
| | OTTHUMP00000039911; cysteine-rich | | | | |
| | secretory protein-3; specific granule | | | | |
| | protein (28 kDa) | | | | |
| PNLIPRP2 | pancreatic lipase-related protein 2 | PLRP2 | | | |
| Map-7 | tumor protein p53 (Li-Fraumeni syndrome; | | | | |
| | p53 tumor suppressor; tumor protein p53 | | | | |
| CK7 related | not found | | | | |
| gene | WW and C2 domain containing 1 | FLJ10865 | FLJ23369 | KIAA0869 | KIBRA |
| AB020676 | | | | | |
| gene | kazrin | KIAA1026 | RP1-21018.1 | | |
| AB028949 | | | | | |
| MAGE-A6 | melanoma antigen, family A, 6 | Magea6 | MGC130207 | MGC151279 | Mage-a6 |
| | [Mus musculus] | | | | |
| MMP-19 | matrix metallopeptidase 19 | MMP19 | MMP18 | RASI-1 | |
| | [Homo sapiens]; matrix | | | | |
| | metalloproteinase 18; matrix | | | | |
| | metalloproteinase 19 | | | | |
| TRIM29 | tripartite motif-containing 29 | ATDC | FLJ36085 | | |
| | [Homo sapiens]; ataxia- | | | | |
| | telangiectasia group D-associated | | | | |
| | protein; tripartite motif protein | | | | |
| | TRIM29 | | | | |
| CEA5 | CEA-related cell adhesion molecule | Ceacam9 | Cea5 | Cea-5 | mmCGMS |
| | 9 [Mus musculus]; | | | | |
| | carcinoembryonic antigen 5 | | | | |
| MSLN | mesothelin [Homo sapiens]; | CAK1 | MPF | SMR | |
| | megakaryocyte potentiating factor | | | | |
| UPAR | plasminogen activator, urokinase | PLAUR | CD87 | URKR | |
| | receptor [Homo sapiens]; monocyte | | | | |
| | activation antigen Mo3; u-plasminogen | | | | |
| | activator receptor form 2 | | | | |

| | | | | | | | | | |
|---|---|---|---|---|---|---|---|---|---|
| mam | | | | | | | | | |
| AGR2 | | | | | | | | | |
| TFF1 | pNR-2 | pS2 | | | | | | | |
| PDEF | | | | | | | | | |
| S100P | | | | | | | | | |
| EpCam1 | EGP40 | Ep-CAM | GA733-2 | KSA | M4S1 | MIC18 | MK-1 | TROP1 | hEGP-2 |
| CK19 | | | | | | | | | |
| CK8 | K8 | KO | | | | | | | |
| Esx | ESX | | | | | | | | |
| CEA6 | | | | | | | | | |
| GPX2 | | | | | | | | | |
| MAL2 | | | | | | | | | |
| Spint2 | | | | | | | | | |
| EpCam2 | M1S1 | | | | | | | | |
| Claudin3 | RVP1 | CLDN3 | | | | | | | |
| NQO1 | NMORI | QR1 | | | | | | | |
| MET | | | | | | | | | |
| MAGE-A3 | MGC14613 | | | | | | | | |
| XAGE-1 | | | | | | | | | |
| KRT81 | ghHkb1 | hHAKB2-1 | | | | | | | |
| Mucin1 | | | | | | | | | |
| FXYD 3 | FXYD | | | | | | | | |
| GPCR5A | | | | | | | | | |
| SCNN1A | SCNN1 | | | | | | | | |
| SGP28 | MGC126588 | dJ442L6.3 | | | | | | | |
| PNLIPRP2 | | | | | | | | | |
| Map-7 | | | | | | | | | |
| CK7 related | | | | | | | | | |
| gene AB020676 | WWC1 | | | | | | | | |
| gene AB028949 | | | | | | | | | |
| MAGE-A6 | | | | | | | | | |
| MMP-19 | | | | | | | | | |
| TRIM29 | | | | | | | | | |
| CEA5 | AA410097 | AW545709 | | | | | | | |
| MSLN | | | | | | | | | |
| UPAR | | | | | | | | | |

Throughout this application, various publications are referenced. The disclosures of these publications in their entireties are hereby incorporated by reference into this application in order to more fully describe the state of the art to which this invention pertains. All publications and patent applications are herein incorporated by reference to the same extent as if each individual publication or patent application was specifically and individually indicated to be incorporated by reference.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for detecting micrometastatic breast cancer in a patient, comprising the steps of:
a) obtaining a cell sample suspected of containing cancerous cells from axillary lymph node tissue of said patient; and
b) determining whether the AGR2 gene is overexpressed in said cell sample compared to AGR2 gene expression in control lymph node tissue cells, wherein overexpression of the AGR2 gene in said cell sample is indicative of the presence of micrometastatic breast cancer in said patient.

2. The method of claim 1, wherein said control lymph node tissue is cervical lymph node tissue.

3. The method of claim 1, wherein said axillary lymph node tissue is sentinel lymph node tissue.

4. The method of claim 1, wherein expression level of the AGR2 gene is determined at the nucleic acid level.

5. The method of claim 4, wherein expression level of the AGR2 gene is determined by real time RT-PCR

6. A method for detecting micrometastatic breast cancer in a patient, comprising the steps of:
a) obtaining a cell sample suspected of containing cancerous cells from axillary lymph node tissue of said patient; and
b) determining whether the TFF1 gene is overexpressed in said cell sample compared to TFF1 gene expression in control lymph node tissue cells, wherein overexpression of the TFF1 gene in said cell sample is indicative of the presence of micrometastatie breast cancer in said patient.

7. The method of claim 6, wherein said control lymph node tissue is cervical lymph node tissue.

8. The method of claim 6, wherein said axillary lymph node tissue is sentinel lymph node tissue.

9. The method of claim 6, wherein expression level of the TFFI gene is determined at the nucleic acid level.

10. The method of claim 9, wherein expression level of the TFF1 gene is determined by real time RT-PCR.

11. A method for predicting the likelihood that a patient diagnosed with breast cancer will respond to hormonal therapy, comprising the steps of:
a) determining the expression level of the AGR2 gene in a cell sample from said patient, said cell sample comprising primary, metastatic, or micrometastatic breast cancer cells; and
b) comparing the expression level of the AGR2 gene to the expression level of a control gene in said cell sample, wherein a higher expression level of said AGR2 gene compared to the expression level of said control gene is indicative of an increased likelihood of response to treatment with hormonal therapy.

12. The method of claim 11, wherein the comparison of expression levels is expressed as a ratio of AGR2 gene expression compared to control gene expression.

13. The method of claim 11, wherein said control gene is TFF1 or EpCam.

14. The method of claim 11, wherein said cell sample is obtained from axillary lymph node tissue.

15. The method of claim 14, wherein said axillary lymph node tissue is sentinel lymph node tissue.

16. The method of claim 11, wherein expression level of the AGR2 gene and control gene are assessed at the nucleic acid level.

17. The method of claim 16, wherein expression level of said AGR2 gene and control gene are determined by real time RT-PCR.

18. A method for detecting metastatic non-small cell lung cancer or micrometastatic non-small cell lung cancer in a patient, comprising the steps of:
a) obtaining a cell sample suspected of containing cancerous cells from mediastinal lymph node tissue of said patient; and
b) determining whether the AGR2 gene is overexpressed in said cell sample compared to control lymph node tissue cells, wherein overexpression of AGR2 is indicative of the presence of metastatic or micrometastatic non-small cell lung cancer in said patient.

19. The method of claim 18, wherein said control lymph node tissue is non-cancerous mediastinal lymph node tissue.

20. The method of claim 18, wherein expression level of the AGR2 gene is determined at the nucleic acid level.

21. The method of claim 20, wherein expression level of the AGR2 gene is determined by real time RT-PCR.

22. A method for predicting decreased probability of survival in a patient diagnosed with early-stage non-small cell lung cancer, comprising the steps of:
a) determining the expression level of the ARG2 gene in a cell sample from said patient, said cell sample comprising primary, metastatic, or micrometastatic non-small cell lung cancer cells; and
b) comparing the expression level of the AGR2 gene to the expression level of a control gene in said cell sample, wherein a higher expression level of said AGR2 gene compared to the expression level of said control gene is indicative of a decreased probability of survival.

23. The method of claim 22, wherein the determination of the expression level of the AGR2 gene is part of a real-time RT-PCR analysis of a multi-marker panel of genes.

24. The method of claim 23, wherein said multi-marker panel of genes includes measurement of expression of the EpCam gene, the PDEF gene, or the S100P gene, or any combination thereof.

25. The method of claim 22, wherein said control gene is β₂-microglobulin.

26. The method of claim 22, wherein said cell sample is obtained from mediastinal lymph node tissue.

27. A kit comprising at least one PCR primer needed to perform amplification of AGR2 selected from the group consisting of a primer comprising the nucleic acid sequence set forth in SEQ ID NO: 1 and a primer comprising the nucleic acid sequence set forth in SEQ ID NO:2.

28. The kit of claim 27, further comprising at least one PCR primer for the amplification of TIFF1, EpCam, PDEF, or S100P, or any combination thereof.

29. The kit of claim 27, wherein said kit further comprises instructions for use in methods for detecting micrometastatic breast cancer.

30. The kit of claim 27, wherein said kit further comprises instructions for use in methods for predicting the likelihood that a patient diagnosed with breast cancer will respond to hormonal therapy.

31. The kit of claim 27, wherein said kit further comprises instructions for use in methods for detecting metastatic non-small cell lung cancer or micrometastatic non-small cell lung cancer.

32. The kit of claim 27, wherein said kit further comprises instructions for use in methods for predicting decreased probability of survival in a patient diagnosed with early-stage non-small cell lung cancer.

33. A method for inhibiting the growth of breast cancer cells or non-small cell lung cancer cells in human tissue, comprising contacting said tissue with an inhibitor that interacts with AGR2 protein, AGR2 DNA, or AGR2 RNA and thereby inhibits AGR2 function.

34. The method of claim 33, wherein the inhibitor is an siRNA, an miRNA, an antisense RNA, an antisense DNA, or an antagonist of the AGR2 protein.

35. The method of claim 34, wherein said antagonist of the AGR2 protein is an anti-AGR2 antibody.

36. A method for identifying a marker indicative of the presence of micrometastatic disease in a patient, said method comprising the steps of:
a) selecting a plurality of candidate markers;
b) diluting a sample of RNA isolated from metastatic tissue into an excess of RNA isolated from non-metastatic tissue at a ratio of at least 1:50 to create a dilution sample;
c) measuring the expression levels of said plurality of candidate markers in a set of samples using immunofluorescence, said set of samples comprising:
i) said dilution sample;
ii) an undiluted sample of RNA isolated from metastatic tissue; and
iii) a sample of RNA isolated from non-metastatic tissue; and
d) selecting a sub-set of markers from said plurality of candidate markers comprising markers for which:
i) an absence of expression was observed in said sample of RNA isolated from non-metastatic tissue;
ii) a fluorescence signal above 500 relative units was observed in said undiluted sample of RNA isolated from metastatic tissue; and
iii) a fluorescence signal was observed in said dilution sample, wherein said sub-set of markers comprises at least one marker for which overexpression is indicative of the presence of micrometastatic disease in said patient.

37. The method of claim 36, wherein said micrometastatic disease is micrometastatic breast cancer.

38. The method of claim 36, wherein said micrometastatic disease is micrometastatic non-small cell lung cancer.

39. A method for detecting metastatic cancer in a patient, comprising the steps of:
a) obtaining a cell sample suspected of containing cancerous cells from lymph node tissue of said patient; and
b) determining whether the EpCAM1 gene, AGR2 gene, CK19 gene, or CK8 gene, or any combination thereof, are overexpressed in said cell sample compared to expression of said genes in control lymph node tissue cells, wherein overexpression of said genes is indicative of the presence of metastatic cancer in said patient, wherein said metastatic cancer is metastatic breast, lung, or pancreatic cancer.

40. A method for detecting metastatic cancer in a patient, comprising the steps of:
a) obtaining a cell sample suspected of containing cancerous cells from lymph node tissue of said patient; and
b) determining whether the Esx gene, the Map 7 gene, the S100P gene, the AGR2 gene, the CEA6 gene, the GPX2 gene, the TFF1 gene, the Mal2 gene, the Spint2 gene, the EpCAM1 gene, the EpCAM2 gene, the CK8 gene, the CK19 gene, or the Claudin3 gene, or any combination thereof, are overexpressed in said cell sample compared to expression of said genes in control lymph node tissue cells, wherein overexpression of said genes is indicative of the presence of metastatic cancer in said patient, wherein said metastatic cancer is metastatic breast, lung, or pancreatic cancer.

41. A method for detecting metastatic non-small cell lung cancer in a patient, comprising the steps of:
a) obtaining a cell sample suspected of containing cancerous cells from lymph node tissue of said patient; and
b) determining whether the EpCAM1 gene, the EpCAM2 gene, the AGR2 gene, the Esx gene, the CK1 9 gene, the CK8 gene, the CEA6 gene, or the Mal2 gene, or any combination thereof, are overexpressed in said cell sample compared to expression of said genes in control lymph node tissue cells, wherein overexpression of said genes is indicative of the presence of metastatic non-small cell lung cancer in said patient.

42. A method for detecting metastatic non-small cell lung cancer in a patient, comprising the steps of:
a) obtaining a cell sample suspected of containing cancerous cells from lymph node tissue of said patient; and
b) determining whether the AGR2 gene, the S100P gene, the CK19 gene, the NQ01 gene, the MET gene, the MAGE-A6 gene, the XAGE-1 gene, the KRTHB1 gene, the MAGE-A3 gene, or the MAP7 gene, or any combination thereof, are overexpressed in said cell sample compared to expression of said genes in control lymph node tissue cells, wherein overexpression of said genes is indicative of the presence of metastatic non-small cell lung cancer in said patient.

43. A method for detecting metastatic breast cancer in a patient, comprising the steps of:
a) obtaining a cell sample suspected of containing cancerous cells from lymph node tissue of said patient; and
b) determining whether the AGR2 gene, the S100P gene, the CK1 9 gene, the Mucin1 gene, the FXYD gene, the Claudin3 gene, the CEA6 gene, the GPCR5A gene, the CK7 related gene, or the SCNNIA gene, or any combination thereof, are overexpressed in said cell sample compared to expression of said genes in control lymph node tissue cells, wherein overexpression of said genes is indicative of the presence of metastatic breast cancer in said patient.

44. A method for detecting metastatic pancreatic cancer in a patient, comprising the steps of:
a) obtaining a cell sample suspected of containing cancerous cells from lymph node tissue of said patient; and
b) determining whether the PNLIPRP2 gene, the CK19 gene, the AGR2 gene, the FXYD gene, the SGP28 gene, the CEA6 gene, the gene of Accession Number AB020676, the Mucin1 gene, the gene of Accession Number AB028949, or the MMP19 gene, or any combination thereof, are overexpressed in said cell sample compared to expression of said genes in control lymph node tissue cells, wherein overexpression of said genes is indicative of the presence of metastatic pancreatic cancer in said patient.

45. A method for detecting metastatic cancer in a patient, comprising the steps of:
a) obtaining a cell sample suspected of containing cancerous cells from lymph node tissue of said patient; and
b) determining whether the Esx gene is overexpressed in said cell sample compared to Esx gene expression in control lymph node tissue cells, wherein overexpression of the Esx gene is indicative of the presence of metastatic cancer in said patient, wherein said metastatic cancer is metastatic breast, lung, or pancreatic cancer.

46. A method for inhibiting the growth of metastatic cancer cells in human tissue, comprising contacting said tissue with an inhibitor that interacts with Esx protein, Esx DNA, or Esx RNA and thereby inhibits Esx function.

47. The method of claim 46, wherein the inhibitor is an siRNA, an miRNA, an antisense RNA, an antisense DNA, or an antagonist of the Esx protein.

48. The method of claim 47, wherein said antagonist of the Esx protein is an anti-Esx antibody.
